(19) United States
(12) Patent Application Publication
(10) Pub. No.: US 2024/0158384 A1
(43) Pub. Date: May 16, 2024

(54) CB2 RECEPTOR AGONISTS

(71) Applicant: The Board of Trustees of the Leland Stanford Junior University, Stanford, CA (US)

(72) Inventors: Michael J. Green, Stanford, CA (US); Alam Jahangir, Stanford, CA (US); Mehrdad Shamloo, Stanford, CA (US); Annelise E. Barron, Stanford, CA (US); Jennifer S. Lin, Stanford, CA (US); David Sperandio, Stanford, CA (US); Amna T. Adam, Stanford, CA (US); Heui Hye Park, Stanford, CA (US); Neir Eshel, Stanford, CA (US)

(21) Appl. No.: 18/257,167

(22) PCT Filed: Dec. 14, 2021
(86) PCT No.: PCT/US21/63360

§ 371 Date: Jun. 13, 2023

Related U.S. Application Data
(60) Provisional application No. 63/125,175, filed on Dec. 14, 2020.
(60) Provisional application No. 63/231,582, filed on Aug. 10, 2021.

(51) Int. Cl. C07D 413/14; A61P 25/16; C07D 401/14; C07D 413/12; C07D 471/04

(52) U.S. Cl. CPC C07D 413/14; A61P 25/16; C07D 401/14; C07D 413/12; C07D 471/04

(57) ABSTRACT

Provided herein are compounds, compositions, and methods for treating disorders (e.g., opioid addiction) using compounds disclosed herein, which are selective agonists of the cannabinoid 2 receptor (CB2R).

### CROSS-REFERENCE TO RELATED APPLICATIONS

This application is a national phase application under 35 U.S.C. § 371 of International Application No. PCT/US2021/063360, filed on Dec. 14, 2021, which claims priority to and the benefit of U.S. Provisional Patent Application No. 63/125,175, filed on Dec. 14, 2020, and U.S. Provisional Patent Application No. 63/231,582, filed on Aug. 10, 2021, each of which is incorporated herein by reference in its entirety.

### STATEMENT REGARDING FEDERALLY SPONSORED RESEARCH

This invention was made with Government support under contract DA052415 awarded by the National Institutes of Health. The Government has certain rights in the invention.

### BACKGROUND

Cannabinoids are a group of compounds found in Cannabis sativa (also known as marijuana), which includes Δ-tetrahydrocannabinol (THC), cannabinol, and cannabidiol. Although Cannabis has been used therapeutically for a variety of illnesses, its use is limited by its psychoactive effects including hallucination, addiction and dependence.

The physiological effects of cannabinoids are mediated by at least two G-protein coupled receptors, CB1R and CB2R. CB1 Rs are expressed primarily in the central nervous system and are found also in peripheral tissues including the immune system, regulate the release of neurotransmitters from the pre-synaptic neurons, and are believed to mediate most of the psychotropic effects of cannabis. CB2 Rs are predominantly found in the immune system in various cell types, including B cells, NK cells, monocytes, microglial cells, neutrophils, T cells, dendritic cells and mast cells, suggesting that a wide range of immune functions can be regulated through CB2R.

Selective activation of CB2R may provide beneficial effects while avoiding the adverse effects seen with dual CB1/CB2 cannabinoid receptor agonists (see, e.g., Fox et al. Expert Opin, Inv. Drug. 2005, 14(6), 695-703).

### SUMMARY

The present disclosure provides compounds of formula (I):

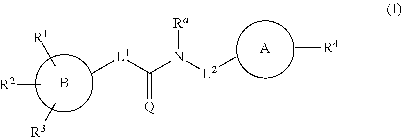

or a pharmaceutically acceptable salt thereof, wherein:
Q is O, S, or NR^{b};
L¹ is a bond or CR^{c}R^{d};
L² is a bond or CR^{e}R^{f};
A is a five- or six-membered heteroaryl having 1, 2, or 3 heteroatoms independently selected from N, O, and S; or a 8- to 10-membered heterocyclyl having 1, 2, or 3 heteroatoms independently selected from N, O, and S; wherein the heteroaryl or the heterocyclyl is optionally substituted with one substituent selected from C₁₋₆ alkyl and C₁₋₆ haloalkyl;
B is a 5- or 6-membered monocyclic heterocyclyl, a 5- or 6-membered monocyclic heteroaryl, or an 8- to 10-membered bicyclic heterocyclyl;
R¹, R², R³, R⁴, and R⁵ are each independently selected from hydrogen, C₁₋₆ alkyl, halo-C₁₋₆-alkyl, C₃₋₇ Cycloalkyl, C₁₋₃-alkyl-C₃₋₇-Cycloalkyl, C₃₋₇-Cycloalkyl-C₁₋₃-alkyl, —C(O)—C₁₋₆ alkyl, —C(O)-heterocyclyl, —NR^{6a}R^{6b}, —NR^{6c}C(O)R^{6d}, oxo, aryl, arylalkyl, heteroaryl, and heterocyclyl, each of which is unsubstituted or substituted with 1-6 substituents selected from halo, halo-C₁₋₃-alkyl, cyano, C₃₋₇ cycloalkyl, —OR^{6e}, —(C₁₋₃ alkyl)-OR^{6f}, —NR^{6g}R^{6h}, —C(O)NR⁶ⁱR^{6j}, and pentafluorosulfanyl;
R^{a}, R^{b}, R^{c}, R^{d}, R^{e}, and R^{f} are each independently selected from hydrogen and C₁₋₃ alkyl; and
R^{6a}, R^{6b}, R^{6c}, R^{6d}, R^{6e}, R^{6a}, R⁶, R^{6h}, R^{6′}, and R^{6j} are each independently selected from hydrogen, C₁₋₆ alkyl, and C₃₋₇ cycloalkyl.

In some embodiments, A is a five-membered heteroaryl having 1, 2, or 3 heteroatoms independently selected from N, O, and S. In some embodiments, the group

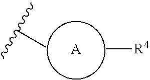

has a formula selected from:

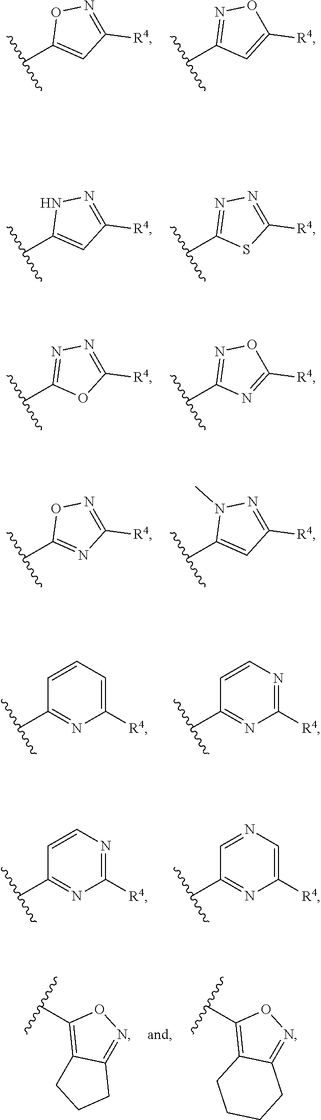

wherein [symbol] represents the point of attachment to L² in formula (I).

In some embodiments, R⁴ is selected from C₁₋₆ alkyl, halo-C₁₋₆-alkyl, C₃₋₇ cycloalkyl, C₁₋₃-alkyl-C₃₋₇-cycloalkyl, aryl, —NR^{6a}R^{6b}, —NR^{6c}C(O)R^{6d}, and heterocyclyl, each of which is unsubstituted or substituted with 1-4 substituents independently selected from halo, —OR^{6e}, —(C₁₋₃ alkyl)-OR^{6f}, —NR^{6g}R^{6h}, and cyano, wherein R^{6e}, R^{6f}, R^{6g}, and R^{6h} are each independently selected from hydrogen and methyl. In some embodiments, R⁴ is selected from:

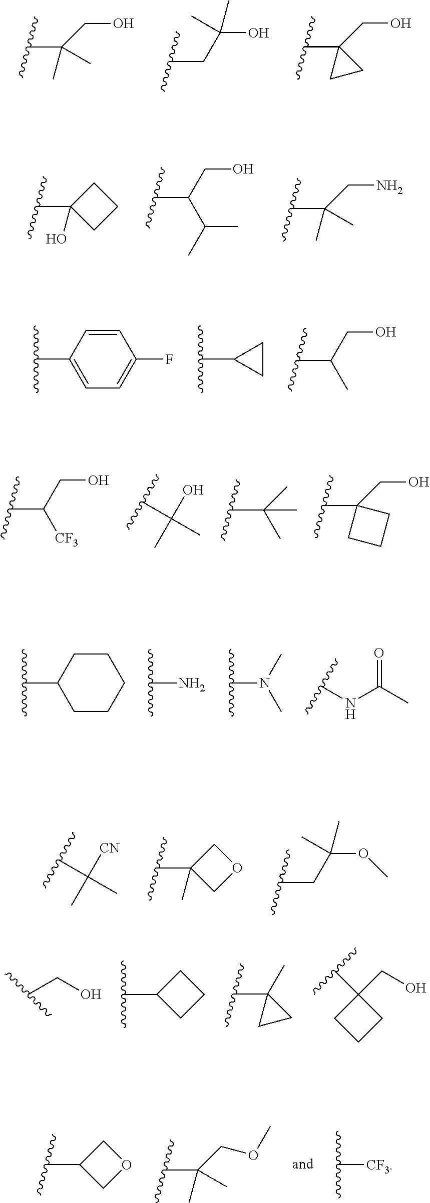

In some embodiments, R^{a} is hydrogen.

In some embodiments, Q is O or NR^{b}, wherein R^{b} is selected from hydrogen and methyl.

In some embodiments, L¹ and L² are each independently selected from a bond and —C(CH₃)₂—.

In some embodiments, B is a monocyclic 5- or 6-membered heterocyclyl having 1 or 2 nitrogen atoms, or a bicyclic 8- to 10-membered heterocyclyl having 1 or 2 nitrogen atoms.

In some embodiments, R² is selected from hydrogen and oxo.

In some embodiments, R³ is selected from hydrogen, C₁₋₆ alkyl, —C(O)CH₃, and —C(O)N(CH₃)₂.

In some embodiments, the group

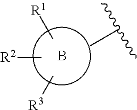

has a structure selected from:

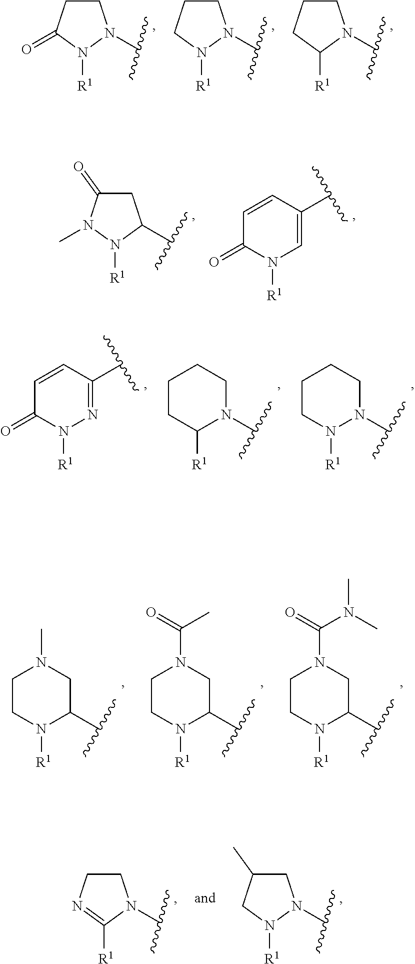

wherein [symbol] represents the point of attachment to L¹ in formula (I).

In some embodiments, R¹ is selected from aryl, heteroaryl, C₃₋₇ cycloalkyl, C₁₋₆ alkyl, C₃₋₇-cycloalkyl-C₁₋₃-alkyl, arylalkyl, heterocyclyl, and —C(O)heterocyclyl, each of which is independently unsubstituted or substituted with 1-3 substituents independently selected from halo, halo-C₁₋₃-alkyl, cyano, C₃₋₇ cycloalkyl, and pentafluorosulfanyl. In some embodiments, R¹ is selected from:

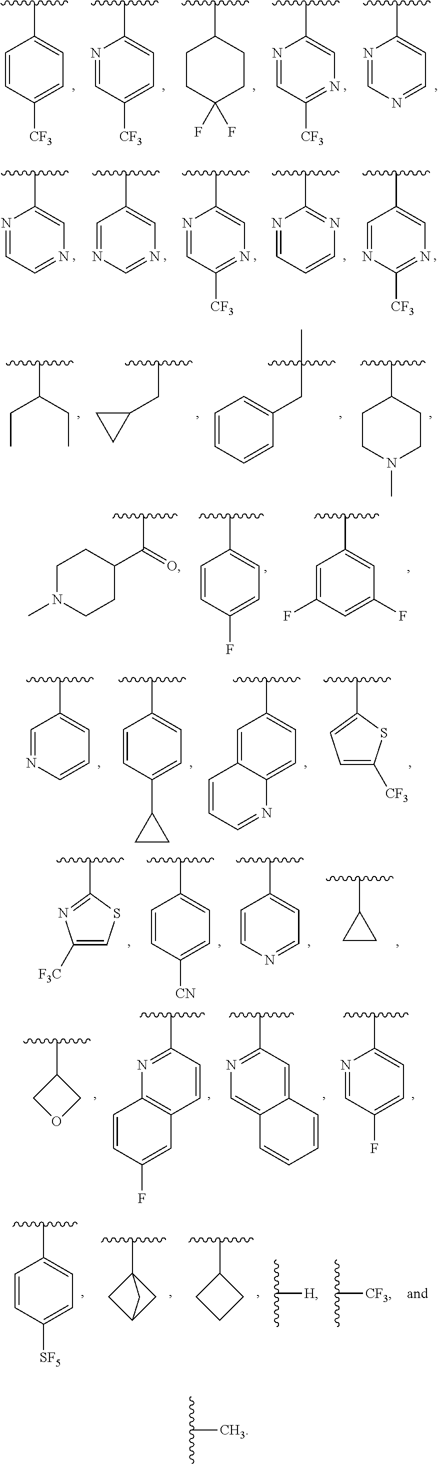

The present disclosure provides compounds of formula (Ia):

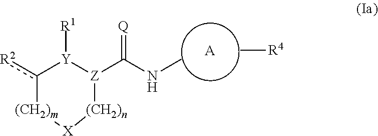

or a pharmaceutically acceptable salt thereof, wherein:
m and n are each independently 1, 2, 3, or 4;
X is a bond, O, NR⁵, or S(O)ₚ;
Y is N or CH;
p is 0, 1, or 2;
Z is CR³ or N;
Q is O, S, or NH;
A is a five- or six-membered heteroaryl having 1, 2, or 3 heteroatoms independently selected from N, O, and S;
the dashed line represents the presence or absence of a bond;
R¹, R², R³, R⁴, and R⁵ are each independently selected from hydrogen, C₁₋₆ alkyl, C₃₋₇ cycloalkyl, C₁₋₃-alkyl-C₃₋₇-cycloalkyl, C₃₋₇-cycloalkyl-C₁₋₃-alkyl, —C(O)—C₁₋₆ alkyl, aryl, and heteroaryl, each of which is unsubstituted or substituted with 1-6 substituents selected from halo, halo-C₁₋₃-alkyl, —OR^{6a}, and —(C₁₋₃ alkyl)-OR^{6b}; wherein, when the dashed line represents the presence of a bond, R² is oxo; and wherein R¹ and R² are optionally taken together with the atoms to which they are attached to form a heterocyclic ring; and R^{6a} and R^{6b} are each independently selected from hydrogen, C₁₋₆ alkyl, and C₃₋₇ cycloalkyl.

In some embodiments, X is a bond, O, NR⁵, S, or SO₂, and R⁵ is selected from hydrogen, C₁₋₆ alkyl, —C(O)—C₁₋₆ alkyl, and aryl.

In some embodiments, m and n are each independently selected from 1 and 2. In some embodiments, m is 1 and n is 1.

In some embodiments, Q is O.

In some embodiments, A is a five-membered heteroaryl having 1, 2, or 3 heteroatoms independently selected from N, O, and S, or a six-membered heteroaryl having 1 or 2 nitrogen atoms. In some embodiments, A is selected from:

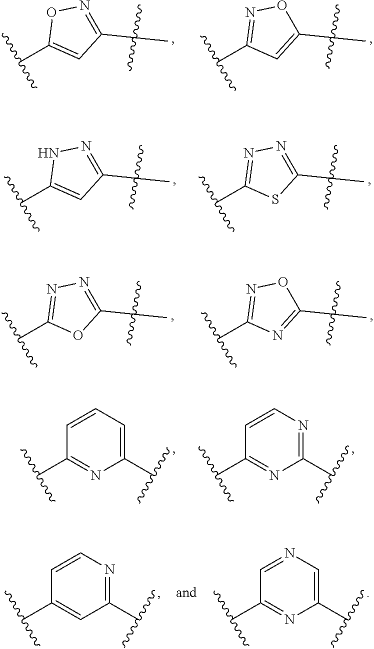

In some embodiments, R¹ is selected from C₁₋₆ alkyl, C₃₋₇ cycloalkyl, C₃₋₇-cycloalkyl-C₁₋₃-alkyl, aryl, and heteroaryl, each of which is unsubstituted or substituted with 1 or 2 substituents selected from halo and halo-C₁₋₃-alkyl. In some embodiments, R¹ is selected from methyl, isopropyl, cyclopropyl, cyclopropylmethyl, phenyl, and pyridyl, wherein the phenyl and pyridyl are optionally substituted with one trifluoromethyl.

In some embodiments, the dashed line represents the absence of a bond, and R² is hydrogen.

In some embodiments, R¹ and R² are taken together with the atoms to which they are attached to form a six-membered heterocyclic ring.

In some embodiments, Z is CR³, and R³ is selected from hydrogen and C₁₋₆ alkyl.

In some embodiments, Y is N.

In some embodiments, R⁴ is selected from C₁₋₆ alkyl, C₃₋₇ cycloalkyl, and C₁₋₃-alkyl-C₃₋₇-cycloalkyl, each of which is unsubstituted or substituted with 1-4 substituents independently selected from halo, —OR^{6a}, and —(C₁₋₃ alkyl)-OR^{6b}. In some embodiments, R⁴ is selected from C₁₋₆ alkyl, C₃₋₄ cycloalkyl, and C₁₋₃-alkyl-C₃₄-cycloalkyl, each of which is unsubstituted or substituted with 1-4 substituents independently selected from fluoro and hydroxy.

In some embodiments, the present disclosure provides a pharmaceutical composition comprising a compound of formula (I) or a compound of formula (Ia), or a pharmaceutically acceptable salt thereof, and a pharmaceutically acceptable carrier.

In some embodiments, the disclosure provides a compound selected from the group consisting of:

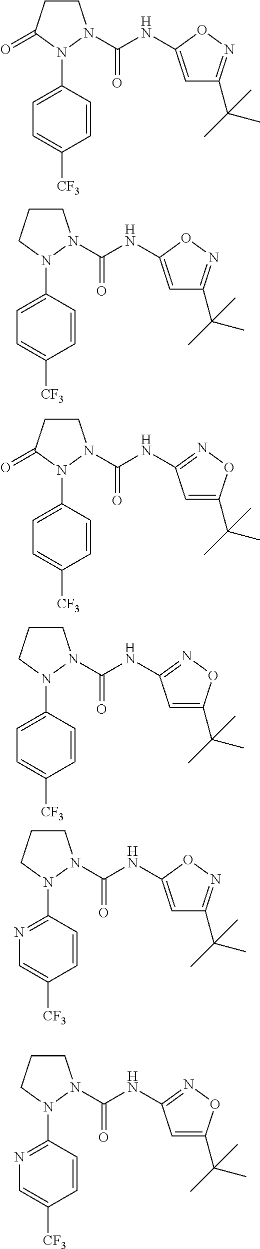

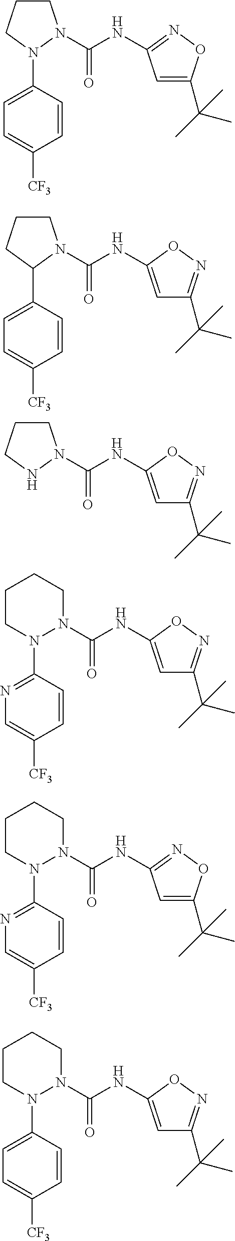

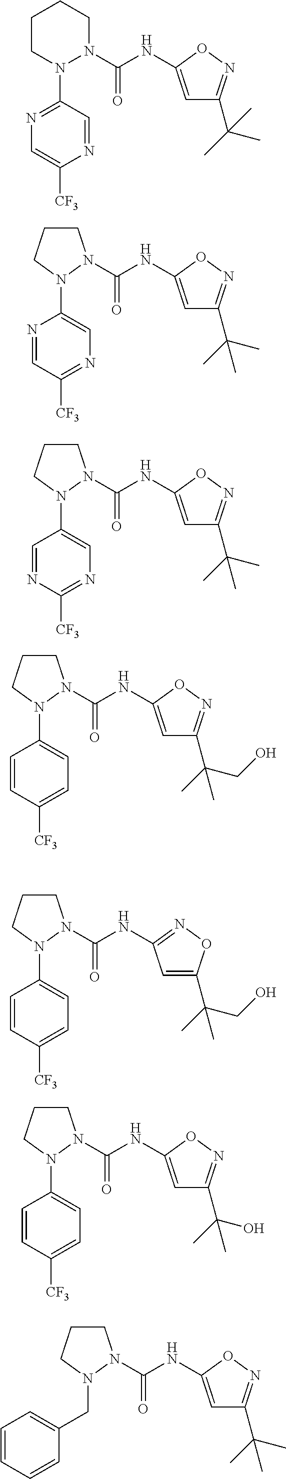

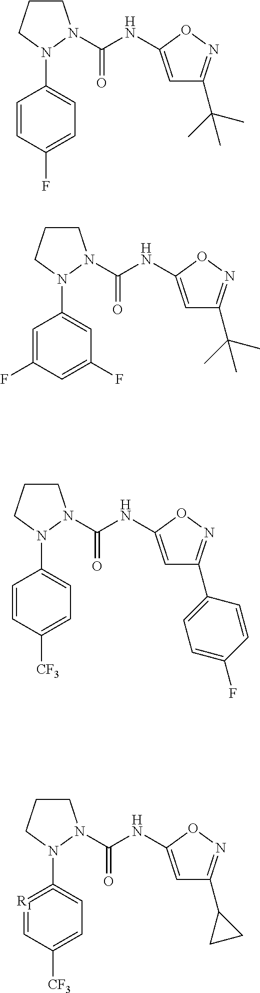

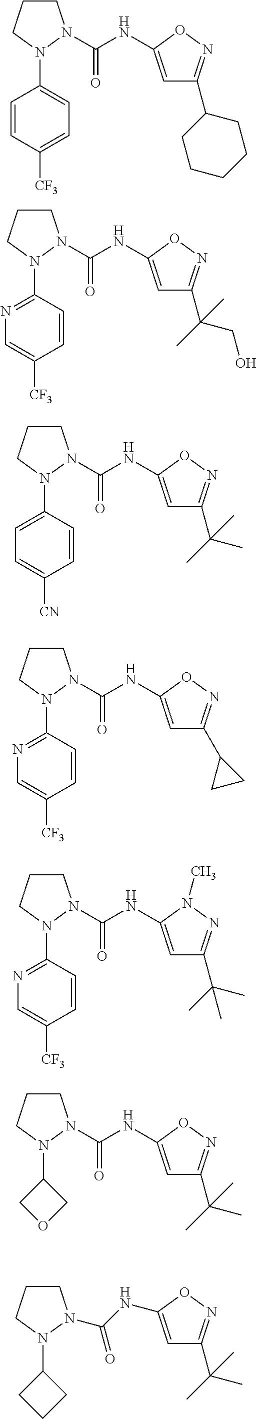

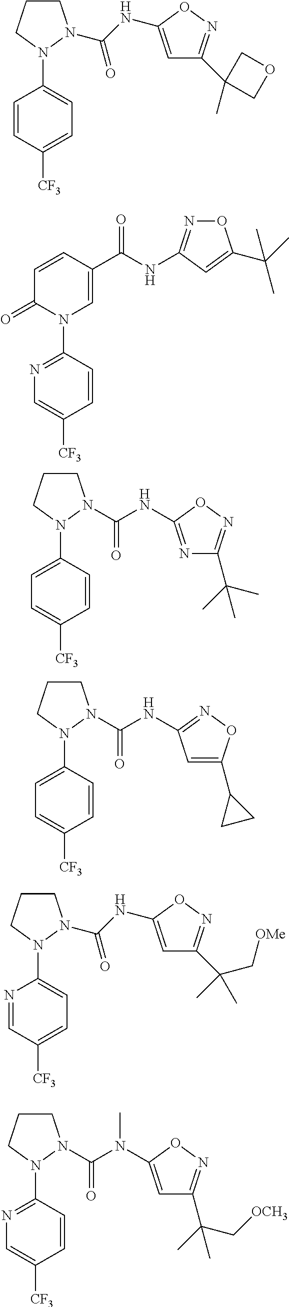

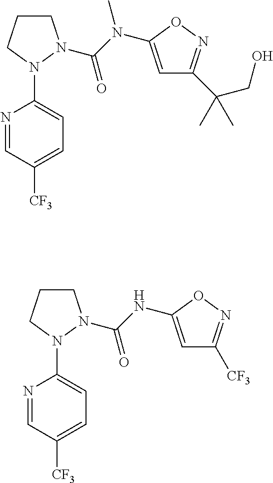 [SMILES: CN(C(=O)N1CCCN1c1ccc(C(F)(F)F)cn1)c1cc(C(C)(C)CO)no1 ; O=C(Nc1cc(C(F)(F)F)no1)N1CCCN1c1ccc(C(F)(F)F)cn1]

and pharmaceutically acceptable salts thereof.

In some embodiments, the present disclosure provides a pharmaceutical composition comprising a compound of formula (I) or a compound of formula (Ia), or a pharmaceutically acceptable salt thereof, and a pharmaceutically acceptable carrier

In some embodiments, the present disclosure provides a method of selectively agonizing a cannabinoid 2 receptor in a subject, comprising administering to the subject an effective amount of a compound of formula (I) or a compound of formula (Ia), or a pharmaceutically acceptable salt thereof, or a pharmaceutical composition comprising a compound of formula (I) or a compound of formula (Ia), or a pharmaceutically acceptable salt thereof.

In some embodiments, the present disclosure provides a method of treating a disorder in a subject in need of treatment, wherein the disorder is selected from addiction, pain, an inflammatory disorder or other disorder having an inflammatory component, a disease having a neuroinflammatory or neurodegenerative component, and Parkinson's disease, comprising administering to the subject a therapeutically effective amount of a compound of formula (I) or a compound of formula (Ia), or a pharmaceutically acceptable salt thereof, or a pharmaceutical composition comprising a compound of formula (I) or a compound of formula (Ia), or a pharmaceutically acceptable salt thereof. In some embodiments, the disorder is an addiction selected from opioid, alcohol, nicotine, methamphetamine, cocaine, and food addiction.

In some embodiments, the compound or composition is co-administered with a cannabinoid receptor antagonist. In some embodiments, the compound or composition is co-administered with a peripherally restricted cannabinoid receptor antagonist. In some embodiments, the compound or composition is co-administered with a centrally active cannabinoid receptor antagonist.

In some embodiments, the present disclosure provides a method of modulating dopaminergic signaling in a subject in need thereof, comprising administering to the subject an effective amount of a compound of formula (I) or a compound of formula (Ia), or a pharmaceutically acceptable salt thereof, or a pharmaceutical composition comprising a compound of formula (I) or a compound of formula (Ia), or a pharmaceutically acceptable salt thereof.

The present disclosure provides a method of manufacturing a compound of formula (I′):

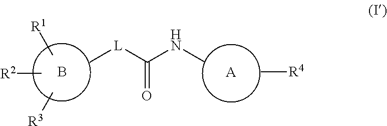

the method comprising:
(a) reacting a compound of formula (Ia′) with a compound of formula (Ib′) in the presence of a coupling reagent:

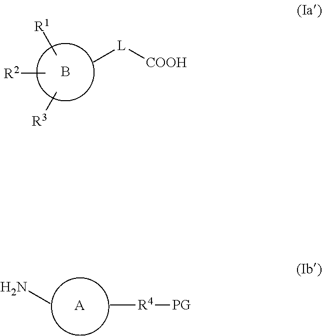

wherein PG is an optional protecting group; and
(b) when PG is present, reacting the product of step (a) with a deprotecting agent;
to thereby manufacture the compound.

In some embodiments, the present disclosure provides a method of manufacturing a compound of formula (Ia):

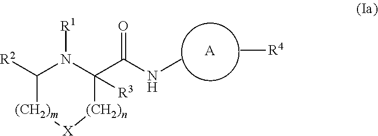

the method comprising:
(a) reacting a compound of formula (Ib) with a compound of formula (Ic) in the presence of a coupling reagent:

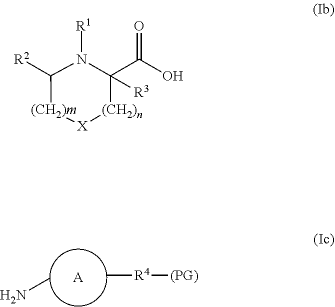

wherein PG is an optional protecting group; and
(b) when PG is present, reacting the product of step (a) with a deprotecting agent;
to thereby manufacture the compound.

The present disclosure provides compounds of formula (II):

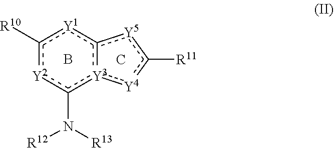

or a pharmaceutically acceptable salt thereof, wherein:
each dashed line represents the presence or absence of a bond, and each is independently chosen such that ring B and ring C are each heteroaryl;
Y¹ is selected from CR^{14a} and N;
Y² is selected from CR^{14b} and N;
Y³ is selected from C and N;
Y⁴ is selected from N, NR^{15a}, CR^{15b}, and S;
Y⁵ is selected from NR^{16a}, CR^{16b}, and S;
R¹⁰ is selected from hydrogen and C₁-C₄ alkyl;
R¹¹ is selected from heteroaryl and aryl, each of which is optionally substituted;
R¹² and R¹³ are each independently selected from hydrogen and C₁-C₆ alkyl, or R¹² and R¹³, together with the nitrogen atom to which they are attached, form an optionally substituted heterocyclic ring; and
R^{14a}, R^{14b}, R^{15a}, and R^{15b} are each independently selected from hydrogen and C₁-C₄ alkyl; and
R^{16a} and R^{16b} are each independently selected from hydrogen, C₁-C₆ alkyl, C₂-C₆ alkenyl, C₂-C₆ alkynyl, aryl, heteroaryl, cycloalkyl, and heterocyclyl, each of which is optionally substituted;
wherein Y¹, Y², Y⁴, and Y⁵ are not all simultaneously N.

In some embodiments, the compound has a structure selected from:

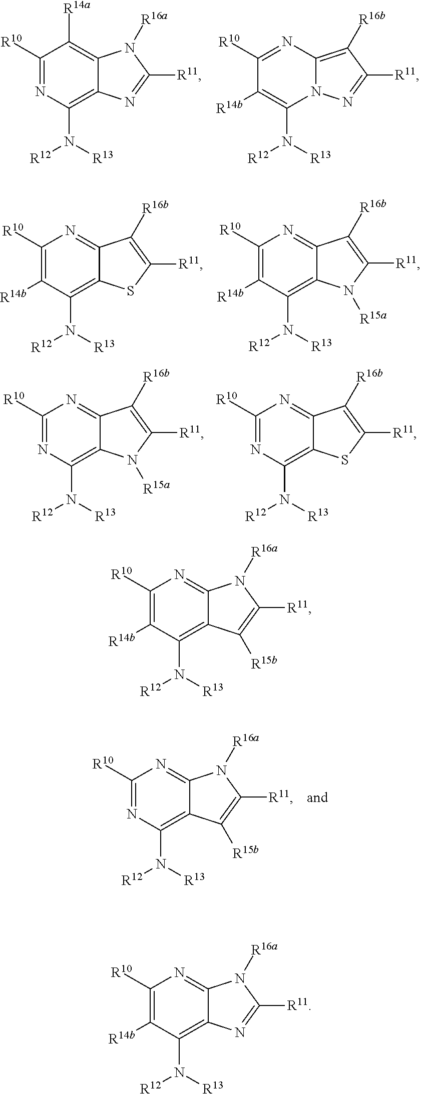

In some embodiments, Y¹ is CR^{14a}, and R^{14a} is hydrogen. In some embodiments, Y¹ is N.

In some embodiments, Y² is CR^{14b}, and R^{14b} is hydrogen. In some embodiments, Y² is N.

In some embodiments, Y³ is C. In some embodiments, Y³ is N.

In some embodiments, Y⁴ is N. In some embodiments, Y⁴ is NR^{15a}, and R^{15a} is methyl. In some embodiments, Y⁴ is CR^{15b}, and R^{15b} is hydrogen. In some embodiments, Y⁴ is S.

In some embodiments, Y⁵ is NR^{16a}, and R^{16a} is monocyclic heterocyclyl. In some embodiments, Y⁵ is CR^{16b}, and R^{16b} is monocyclic heterocyclyl.

In some embodiments, R¹⁰ is selected from hydrogen and methyl. In some embodiments, R¹⁰ is methyl.

In some embodiments, R¹¹ is aryl. In some embodiments, R¹¹ is phenyl substituted with 1 or 2 substituents independently selected from halo, C₁-C₄ alkyl, and C₁-C₄ alkoxy.

In some embodiments, R¹² and R¹³, together with the nitrogen atom to which they are attached, form a monocyclic 5-6 membered heterocyclic ring optionally substituted with 1 or 2 substituents independently selected from C₁-C₄ alkyl, halo, and C₁-C₄ alkoxy. In some embodiments, R¹² and R¹³, together with the nitrogen atom to which they are attached, form a 6-membered heterocyclic ring having 1 or 2 nitrogen atoms, wherein the heterocyclic ring is substituted with 1 substituent independently selected from C₁-C₄ alkyl, halo, and C₁-C₄ alkoxy.

In some embodiments, the compound of formula (II) is selected from:

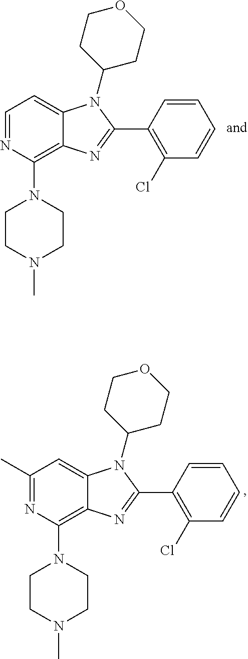 [SMILES: CN1CCN(c2nccc3c2nc(-c2ccccc2Cl)n3C2CCOCC2)CC1 ; Cc1cc2c(nc(-c3ccccc3Cl)n2C2CCOCC2)c(N2CCN(C)CC2)n1]

and pharmaceutically acceptable salts thereof.

In some embodiments, the present disclosure provides a pharmaceutical composition comprising a compound of formula (II), or a pharmaceutically acceptable salt thereof, and a pharmaceutically acceptable carrier.

In some embodiments, the present disclosure provides a method of selectively agonizing a cannabinoid 2 receptor in a subject, comprising administering to the subject an effective amount of a compound of formula (II) or a pharmaceutically acceptable salt thereof, or a pharmaceutical composition comprising a compound of formula (II) or a pharmaceutically acceptable salt thereof.

In some embodiments, the present disclosure provides a method of treating a disorder in a subject in need of treatment, wherein the disorder is selected from addiction, pain, an inflammatory disorder or other disorder having an inflammatory component, a disease having a neuroinflammatory or neurodegenerative component, and Parkinson's disease, comprising administering to the subject a therapeutically effective amount of a compound of formula (II) or a pharmaceutically acceptable salt thereof, or a pharmaceutical composition comprising a compound of formula (II) or a pharmaceutically acceptable salt thereof. In some embodiments, the disorder is an addiction selected from opioid, alcohol, nicotine, methamphetamine, cocaine, and food addiction.

In some embodiments, the compound or composition is co-administered with a cannabinoid receptor antagonist. In some embodiments, the compound or composition is co-administered with a peripherally restricted cannabinoid receptor antagonist. In some embodiments, the compound or composition is co-administered with a centrally active cannabinoid receptor antagonist.

In some embodiments, the present disclosure provides a method of modulating dopaminergic signaling in a subject in need thereof, comprising administering to the subject an effective amount of a compound of formula (II) or a pharmaceutically acceptable salt thereof, or a pharmaceutical composition comprising a compound of formula (II) or a pharmaceutically acceptable salt thereof.

In some embodiments, the present disclosure provides a method of manufacturing a compound of formula (II), wherein the compound is a compound of formula (IIa);

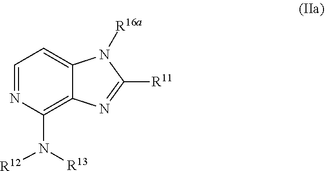

the method comprising:
(a) reacting 2,4-dichloro-3-nitropyridine with a compound R^{16a}—NH₂ in the presence of a base to generate a compound of formula (IIb):

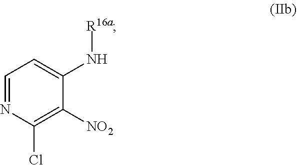

(b) reacting the compound of formula (IIb) with a compound HNR12R13 in the presence of base to generate a compound of formula (IIc):

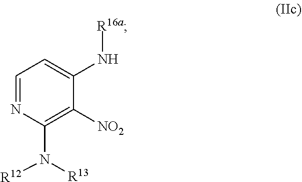

(c) reducing the compound of formula (IIc) to generate a compound of formula (IId):

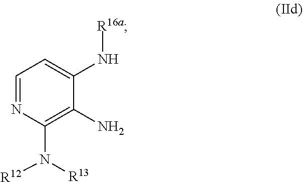

and
(d) reacting the compound of formula (Id) with a compound R¹¹-CHO in the presence of sodium metabisulfite to thereby synthesize the compound of formula (IIa).

### BRIEF DESCRIPTION OF THE DRAWINGS

FIGS. 1A-1C show: (1A) inhibition of accumulation of forskolin-stimulated cAMP levels by LY2828360 and CP-55940 in CHO cells stably expressing hCB1 receptor (top) or hCB2 receptor (bottom); (1B) inhibition of accumulation of forskolin-stimulated cAMP levels by Reference Compound 22 and CP-55940 in CHO cells stably expressing hCB1 receptor (top) or hCB2 receptor (bottom); (1C) beta arrestin recruitment at the hCB2 receptor in the presence of CP-55940, Reference Compound 22, or LY2828360.

FIGS. 2A-2B show: (2A) inhibition of accumulation of forskolin-stimulated cAMP levels by Compound 11 and CP-55940 in CHO cells stably expressing hCB1 receptor (top) or hCB2 receptor (bottom); (2B) beta arrestin recruitment at the hCB2 receptor in the presence of CP-55940 or Compound 11.

FIGS. 3A-3B show: (3A) inhibition of accumulation of forskolin-stimulated cAMP levels by Compound 22 and CP-55940 in CHO cells stably expressing hCB1 receptor (top) or hCB2 receptor (bottom); (3B) beta arrestin recruitment at the hCB2 receptor in the presence of CP-55940 or Compound 22.

FIGS. 4A-4B show: (4A) inhibition of accumulation of forskolin-stimulated cAMP levels by Compound 16 and CP-55940 in CHO cells stably expressing hCB1 receptor (top) or hCB2 receptor (bottom); (4B) beta arrestin recruitment at the hCB2 receptor in the presence of CP-55940 or Compound 16.

FIGS. 5A-5B show: (5A) inhibition of accumulation of forskolin-stimulated cAMP levels by Compound 24 and CP-55940 in CHO cells stably expressing hCB1 receptor (top) or hCB2 receptor (bottom); (5B) beta arrestin recruitment at the hCB2 receptor in the presence of CP-55940 or Compound 24.

FIGS. 6A-6B show data from a heroin self-administration assay: (6A) a timeline of the experiment; (6B) data from animals treated with LY2828360 (3 mg/kg, i.p.) during the 21 days of abstinence, showing a decrease in heroin seeking behavior compared to controls.

FIG. 7 shows data from a naloxone-precipitated withdrawal model, where LY2828360 treatment reduced the negative effective of naloxone-precipitated withdrawal in heroin-addicted mice.

FIGS. 8A-8B show data demonstrating that Compound 22 does not affect spontaneous locomotor activity in mice (FIG. 8A), and data from a hyperactivity model in mice (FIG. 8B).

FIGS. 9A-9B show data demonstrating that Compound 24 does not affect spontaneous locomotor activity in mice (FIG. 9A), and data from a hyperactivity model in mice (FIG. 9B).

FIGS. 10A-10D show plasma and brain concentrations after IV and PO dosing in mice (n=3/timepoint) at 10 mg/kg, for Compounds 11 (FIG. 10A), 22 (FIG. 10B), 16 (FIG. 10C), and 24 (FIG. 10D).

FIGS. 11A-11F show: a microdialysis schematic (FIG. 11A); data showing that Compound 22 appears to reduce extracellular dopamine (DA) release after AMPH dosing (n=1/group) (FIG. 11B); a schematic of GRAB-DA AAV injection (FIG. 11C); rate of spontaneous DA transients before and after injection of vehicle or Compound 22 (FIG. 11D); shape of average DA transient before and after Compound 22 injection (FIG. 11E); and example photometry recordings from a mouse treated with vehicle (left) or Compound 22 (right) before performing an operant conditioning task for sucrose reward (FIG. 11F).

FIGS. 12A-12B show data demonstrating that Compound 24 displays anti-inflammatory function in a LPS model of systemic inflammation (FIG. 12A), and that Compound 24 displays anti-inflammatory function in the brain.

FIGS. 13A-13D show data for compounds disclosed herein.

### DETAILED DESCRIPTION

The present disclosure relates to compounds that selectively bind to the cannabinoid 2 receptor (CB2R) compared to the cannabinoid 1 receptor (CB1R). The disclosure also relates to pharmaceutical compositions comprising the compounds, and methods of using the compounds and compositions in the treatment of disorders such as addiction (e.g., opioid addiction).

### Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents, and other references mentioned herein are incorporated by reference in their entirety. The materials, methods, and examples disclosed herein are illustrative only and not intended to be limiting.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Sorrell, Organic Chemistry, 2^{nd} edition, University Science Books, Sausalito, 2006; Smith, March's Advanced Organic Chemistry: Reactions, Mechanism, and Structure, 7^{th} Edition, John Wiley & Sons, Inc., New York, 2013; Larock, Comprehensive Organic Transformations, 3^{rd} Edition, John Wiley & Sons, Inc., New York, 2018; and Carruthers, Some Modern Methods of Organic Synthesis, 3^{rd} Edition, Cambridge University Press, Cambridge, 1987; the entire contents of each of which are incorporated herein by reference.

As used herein, the term “alkyl” means a straight or branched saturated hydrocarbon chain containing from 1 to 30 carbon atoms, for example 1 to 16 carbon atoms (C₁-C₁₆ alkyl), 1 to 14 carbon atoms (C₁-C₁₄ alkyl), 1 to 12 carbon atoms (C₁-C₁₂ alkyl), 1 to 10 carbon atoms (C₁-C₁₀ alkyl), 1 to 8 carbon atoms (C₁-C₈ alkyl), 1 to 6 carbon atoms (C₁-C₆ alkyl), 1 to 4 carbon atoms (C₁-C₄ alkyl), 6 to 20 carbon atoms (C₆-C₂₀ alkyl), or 8 to 14 carbon atoms (C₅-C₁₄ alkyl). Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, n-undecyl, and n-dodecyl.

As used herein, the term “alkoxy” refers to an alkyl group, as defined herein, appended to the parent molecular moiety through an oxygen atom. Representative examples of alkoxy include, but are not limited to, methoxy, ethoxy, propoxy, 2-propoxy, butoxy and tert-butoxy.

As used herein, the term “aryl” refers to an aromatic carbocyclic ring system having a single ring (monocyclic) or multiple rings (bicyclic or tricyclic) including fused ring systems, and zero heteroatoms. As used herein, aryl contains 6-20 carbon atoms (C₆-C₂₀ aryl), 6 to 14 ring carbon atoms (C₆-C₁₄ aryl), 6 to 12 ring carbon atoms (C₆-C₁₂ aryl), or 6 to 10 ring carbon atoms (C₆-C₁₀ aryl). Representative examples of aryl groups include, but are not limited to, phenyl, naphthyl, anthracenyl, and phenanthrenyl.

As used herein, the term “cycloalkyl” refers to a saturated carbocyclic ring system containing three to ten carbon atoms and zero heteroatoms. The cycloalkyl may be monocyclic, bicyclic, bridged, fused, or spirocyclic. Representative examples of cycloalkyl include, but are not limited to, cyclopropyl, cyclobutyl, cyclopentyl, cyclohexyl, cycloheptyl, cyclooctyl, cyclononyl, cyclodecyl, adamantyl, bicyclo[1.1.1]pentanyl, bicyclo[2.2.1]heptanyl, bicyclo[3.2.1]octanyl, and bicyclo[5.2.0]nonanyl.

As used herein, the term “halogen” or “halo” means F, Cl, Br, or I.

As used herein, the term “haloalkyl” means an alkyl group, as defined herein, in which at least one hydrogen atom (e.g., one, two, three, four, five, six, seven or eight hydrogen atoms) is replaced by a halogen. Representative examples of haloalkyl include, but are not limited to, fluoromethyl, difluoromethyl, trifluoromethyl, and 2,2,2-trifluoroethyl.

As used herein, the term “heteroaryl” refers to an aromatic group having a single ring (monocyclic) or multiple rings (bicyclic or tricyclic) having one or more ring heteroatoms independently selected from O, N, and S. The aromatic monocyclic rings are five- or six-membered rings containing at least one heteroatom independently selected from O, N, and S (e.g. 1, 2, 3, or 4 heteroatoms independently selected from O, N, and S). The five-membered aromatic monocyclic rings have two double bonds, and the six-membered aromatic monocyclic rings have three double bonds. The bicyclic heteroaryl groups are exemplified by a monocyclic heteroaryl ring appended fused to a monocyclic aryl group, as defined herein, or a monocyclic heteroaryl group, as defined herein. The tricyclic heteroaryl groups are exemplified by a monocyclic heteroaryl ring fused to two rings independently selected from a monocyclic aryl group, as defined herein, and a monocyclic heteroaryl group as defined herein. Representative examples of monocyclic heteroaryl include, but are not limited to, pyridinyl (including pyridin-2-yl, pyridin-3-yl, pyridin-4-yl), pyrimidinyl, pyrazinyl, pyridazinyl, pyrrolyl, benzopyrazolyl, 1,2,3-triazolyl, 1,3,4-thiadiazolyl, 1,2,4-thiadiazolyl, 1,3,4-oxadiazolyl, 1,2,4-oxadiazolyl, imidazolyl, thiazolyl, isothiazolyl, thienyl, furanyl, oxazolyl, isoxazolyl, 1,2,4-triazinyl, and 1,3,5-triazinyl. Representative examples of bicyclic heteroaryl include, but are not limited to, benzimidazolyl, benzodioxolyl, benzofuranyl, benzooxadiazolyl, benzopyrazolyl, benzothiazolyl, benzothienyl, benzotriazolyl, benzoxadiazolyl, benzoxazolyl, chromenyl, imidazopyridine, imidazothiazolyl, indazolyl, indolyl, isobenzofuranyl, isoindolyl, isoquinolinyl, naphthyridinyl, purinyl, pyridoimidazolyl, quinazolinyl, quinolinyl, quinoxalinyl, thiazolopyridinyl, thiazolopyrimidinyl, thienopyrrolyl, and thienothienyl. Representative examples of tricyclic heteroaryl include, but are not limited to, dibenzofuranyl and dibenzothienyl. The monocyclic, bicyclic, and tricyclic heteroaryls are connected to the parent molecular moiety through any carbon atom or any nitrogen atom contained within the rings.

As used herein, the term “heterocycle” or “heterocyclic” refers to a saturated or partially unsaturated non-aromatic cyclic group having one or more ring heteroatoms independently selected from O, N, and S. means a monocyclic heterocycle, a bicyclic heterocycle, or a tricyclic heterocycle. The monocyclic heterocycle is a three-, four-, five-, six-, seven-, or eight-membered ring containing at least one heteroatom independently selected from O, N, and S. The three- or four-membered ring contains zero or one double bond, and one heteroatom selected from O, N, and S. The five-membered ring contains zero or one double bond and one, two or three heteroatoms selected from O, N and S. The six-membered ring contains zero, one, or two double bonds and one, two, or three heteroatoms selected from O, N, and S. The seven- and eight-membered rings contains zero, one, two, or three double bonds and one, two, or three heteroatoms selected from O, N, and S. Representative examples of monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, oxetanyl, piperazinyl, piperidinyl, pyranyl, pyrazolinyl, pyrazolidinyl, pyrrolinyl, pyrrolidinyl, tetrahydrofuranyl, tetrahydropyranyl, tetrahydropyridinyl, tetrahydrothienyl, thiadiazolinyl, thiadiazolidinyl, 1,2-thiazinanyl, 1,3-thiazinanyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to a phenyl group, or a monocyclic heterocycle fused to a monocyclic cycloalkyl, or a monocyclic heterocycle fused to a monocyclic cycloalkenyl, or a monocyclic heterocycle fused to a monocyclic heterocycle, or a spiro heterocycle group, or a bridged monocyclic heterocycle ring system in which two non-adjacent atoms of the ring are linked by an alkylene bridge of 1, 2, 3, or 4 carbon atoms, or an alkenylene bridge of two, three, or four carbon atoms. Representative examples of bicyclic heterocycles include, but are not limited to, benzopyranyl, benzothiopyranyl, chromanyl, 2,3-dihydrobenzofuranyl, 2,3-dihydrobenzothienyl, 2,3-dihydroisoquinoline, 2-azaspiro[3.3]heptan-2-yl, azabicyclo[2.2.1]heptyl (including 2-azabicyclo[2.2.1]hept-2-yl), 2,3-dihydro-1H-indolyl, isoindolinyl, octahydrocyclopenta[c]pyrrolyl, octahydropyrrolopyridinyl, and tetrahydroisoquinolinyl. Tricyclic heterocycles are exemplified by a bicyclic heterocycle fused to a phenyl group, or a bicyclic heterocycle fused to a monocyclic cycloalkyl, or a bicyclic heterocycle fused to a monocyclic cycloalkenyl, or a bicyclic heterocycle fused to a monocyclic heterocycle, or a bicyclic heterocycle in which two non-adjacent atoms of the bicyclic ring are linked by an alkylene bridge of 1, 2, 3, or 4 carbon atoms, or an alkenylene bridge of two, three, or four carbon atoms. Examples of tricyclic heterocycles include, but are not limited to, octahydro-2,5-epoxypentalene, hexahydro-2H-2,5-methanocyclopenta[b]furan,hexahydro-1H-1,4-methanocyclopenta[c]furan, aza-adamantane (1-azatricyclo[3.3.1.1³-7]decane), and oxa-adamantane (2-oxatricyclo[3.3.1.1³-7]decane). The monocyclic, bicyclic, and tricyclic heterocycles are connected to the parent molecular moiety through any carbon atom or any nitrogen atom contained within the rings.

As used herein, the term “hydroxy” means an —OH group.

As used herein, the term “substituent” refers to a group substituted on an atom of the indicated group. When a group or moiety can be substituted, the term “substituted” indicates that one or more (e.g., 1, 2, 3, 4, 5, or 6; in some embodiments 1, 2, or 3; and in other embodiments 1 or 2) hydrogens on the group indicated in the expression using “substituted” can be replaced with a selection of recited indicated groups or with a suitable group known to those of skill in the art (e.g., one or more of the groups recited below), provided that the designated atom's normal valence is not exceeded. Substituent groups include, but are not limited to, alkyl, alkenyl, alkynyl, alkoxy, acyl, amino, amido, amidino, aryl, azido, carbamoyl, carboxyl, carboxyl ester, cyano, cycloalkyl, cycloalkenyl, guanidino, halo, haloalkyl, haloalkoxy, heteroalkyl, heteroaryl, heterocyclyl, hydroxy, hydrazino, imino, oxo, nitro, phosphate, phosphonate, sulfonic acid, thiol, thione, or combinations thereof.

The terms “administer,” “administering,” “administered,” or “administration” refer to any manner of providing a compound or a pharmaceutical composition (e.g., one described herein), to a subject or patient. Routes of administration can be accomplished through any means known by those skilled in the art. Such means include, but are not limited to, oral, buccal, intravenous, subcutaneous, intramuscular, transdermal, by inhalation and the like.

“Effective amount,” as used herein, refers to a dosage of a compound or a composition effective for eliciting a desired effect. This term as used herein may also refer to an amount effective at bringing about a desired in vivo effect in a subject, such as a human.

As used herein, the term “subject” is intended to include human and non-human animals. Exemplary human subjects include a human patient having a disorder, e.g., an opioid addiction. The term “non-human animals” includes all vertebrates, e.g., non-mammals (such as chickens, amphibians, reptiles) and mammals, such as non-human primates, domesticated and/or agriculturally useful animals (such as sheep, dogs, cats, cows, pigs, etc.), and rodents (such as mice, rats, hamsters, guinea pigs, etc.).

As used herein, the term “treat” or “treating” a subject having a disorder refers to administering a compound or a composition described herein to the subject, such that at least one symptom of the disorder is cured, healed, alleviated, relieved, altered, remedied, ameliorated, or improved. Treating includes administering an amount effective to alleviate, relieve, alter, remedy, ameliorate, cure, improve or affect the disorder or the symptoms of the disorder. The treatment may inhibit deterioration or worsening of a symptom of a disorder.

Compounds

The present disclosure provides a compound of formula (I):

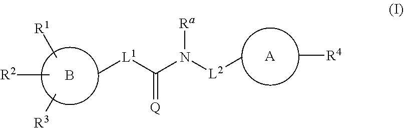

or a pharmaceutically acceptable salt thereof, wherein:
Q is O, S, or NR^{b};
L¹ is a bond or CR^{c}R^{d};
L² is a bond or CR^{e}R^{f};
A is a five- or six-membered heteroaryl having 1, 2, or 3 heteroatoms independently selected from N, O, and S; or a 8- to 10-membered heterocyclyl having 1, 2, or 3 heteroatoms independently selected from N, O, and S; wherein the heteroaryl or the heterocyclyl is optionally substituted with one substituent selected from C₁₋₆ alkyl and C₁₋₆ haloalkyl;
B is a 5- or 6-membered monocyclic heterocyclyl, a 5- or 6-membered monocyclic heteroaryl, or an 8- to 10-membered bicyclic heterocyclyl; R¹, R², R³, R⁴, and R⁵ are each independently selected from hydrogen, C₁₋₆ alkyl, halo-C₁₋₆-alkyl, C₃₋₇ cycloalkyl, C₁₋₃-alkyl-C₃₋₇-cycloalkyl, C₃₋₇-cycloalkyl-C₁₋₃-alkyl, —C(O)—C₁₋₆ alkyl, —C(O)-heterocyclyl, —NR^{6a}R^{6b}, NR^{6c}C(O)R^{6d}, oxo, aryl, arylalkyl, heteroaryl, and heterocyclyl, each of which is unsubstituted or substituted with 1-6 substituents selected from halo, halo-C₁₋₃-alkyl, cyano, C₃₋₇ cycloalkyl, —OR^{6e}, —(C₁₋₃ alkyl)-OR^{6f}, —NR^{6g}, R^{6h}, —C(O)NR⁶ⁱR^{6j}, and pentafluorosulfanyl;
R^{a}, R^{b}, R^{c}, R^{d}, R^{e}, and R^{f} are each independently selected from hydrogen and C₁₋₃ alkyl;
R^{6a}, R^{6b}, R^{6c}, R^{6d}, R^{6e}, R^{6f}, R^{6g}, R^{6h}, R⁶ⁱ, and R^{6j} are each independently selected from hydrogen, C₁₋₆ alkyl, and C₃₋₇ cycloalkyl.

In some embodiments, A is a five-membered heteroaryl having 1, 2, or 3 heteroatoms independently selected from N, O, and S, or a six-membered heteroaryl having 1 or 2 nitrogen atoms. In some embodiments, A is a five-membered heteroaryl having 1, 2, or 3 heteroatoms independently selected from N, O, and S. In some embodiments, A is a six-membered heteroaryl having 1 or 2 nitrogen atoms. In some embodiments, A is selected from isoxazole, pyrazole, thiadiazole (e.g., 1,2,4-thiadiazole or 1,3,4-thiadiazole), oxadiazole (e.g., 1,2,4-oxadiazole or 1,3,4-oxadiazole), pyridine, pyridazine, pyrimidine, and pyrazine. In some embodiments, the group

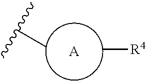

has a formula selected from:

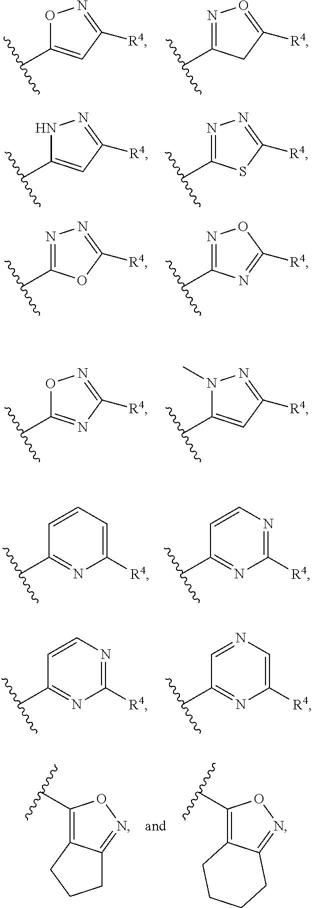

wherein [symbol] represents the point of attachment to L² in formula (I).

In some embodiments, the group

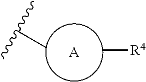

has a formula selected from:

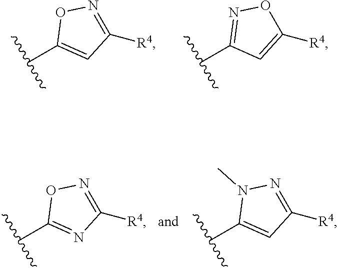

wherein [symbol] represents the point of attachment to L² in formula (I).

In some embodiments, R⁴ is selected from C₁₋₆ alkyl, halo-C₁₋₆-alkyl, C₃₋₇ cycloalkyl, C₁₋₃-alkyl-C₃₋₇-cycloalkyl, aryl, —NR^{6a}R^{6b}, —NR^{6c}C(O)R^{6d}, and heterocyclyl, each of which is unsubstituted or substituted with 1-4 substituents independently selected from halo, —OR^{6e}, —(C₁₋₃ alkyl)-OR⁶¹, —NR^{6g}R^{6h}, and cyano, wherein R^{6e}, R^{6f}, R^{6g}, and R^{6h} are each independently selected from hydrogen and methyl. In some embodiments, R⁴ is C₁₋₆ alkyl or halo-C₁₋₆-alkyl, each of which is unsubstituted or substituted with 1 substituent independently selected from hydroxy, methoxy, and —NH₂. In some embodiments, R⁴ is C₁₋₆ alkyl (e.g., ethyl, isopropyl, tert-butyl, isobutyl, or 3-methylbutan-2-yl), substituted with one hydroxy group. In some embodiments, R⁴ is C₃₋₆ cycloalkyl (e.g., cyclopropyl, cyclobutyl, or cyclohexyl), optionally substituted with one hydroxy group or one hydroxymethyl group. In some embodiments, R⁴ is C₁₋₃-alkyl-C₃₋₇-cycloalkyl (e.g., methylcyclopropyl) substituted with one hydroxy group. In some embodiments, R⁴ is unsubstituted C₁₋₆ alkyl (e.g., tert-butyl).

In some embodiments, R⁴ is selected from:

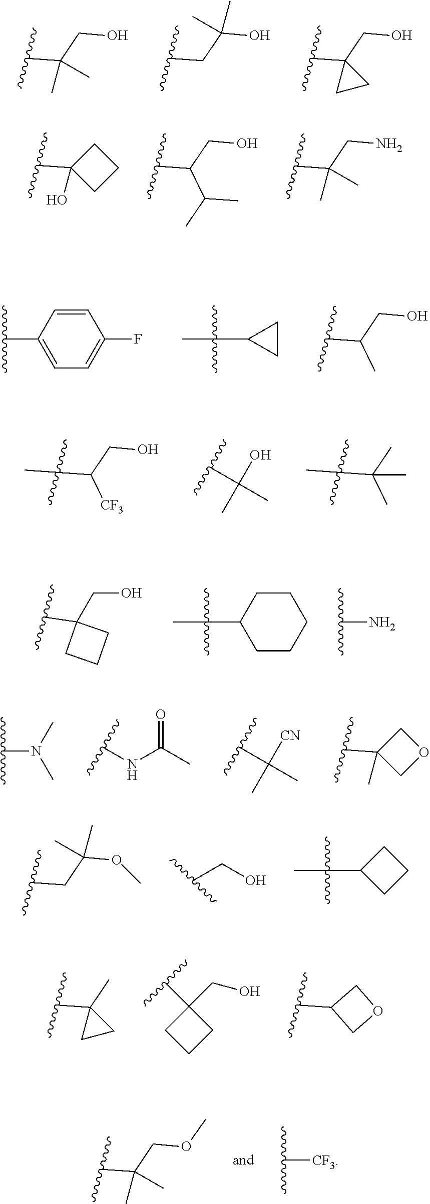

In some embodiments, R⁴ is selected from:

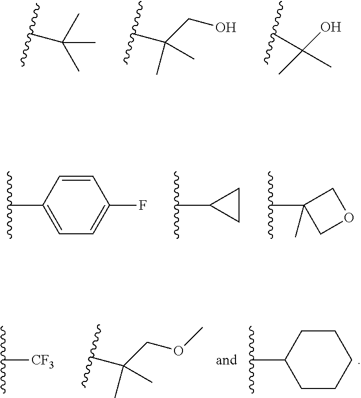

In some embodiments, R^{a} is hydrogen.

In some embodiments, Q is O or NR^{b}. In some embodiments, Q is O, NH, or N(CH₃). In some embodiments, Q is O. In some embodiments, Q is NH. In some embodiments, Q is N(CH₃). In some embodiments, Q is S.

In some embodiments, L¹ is a bond. In some embodiments, L¹ is —C(CH₃)₂—. In some embodiments, L² is a bond. In some embodiments, L² is —C(CH₃)₂—.

In some embodiments, B is a monocyclic 5- or 6-membered heterocyclyl having 1 or 2 nitrogen atoms. In some embodiments, B is selected from pyrrolidinyl, pyrazolidinyl, piperidinyl, hexahydropyridazinyl, dihydropyridinyl, dihydropyridazinyl, piperazinyl, and dihydroimidazolyl. In some embodiments, R² is selected from hydrogen and oxo. In some embodiments, R³ is selected from hydrogen, C₁₋₆ alkyl (e.g., methyl), —C(O)CH₃, and —C(O)N(CH₃)₂.

In some embodiments, the group

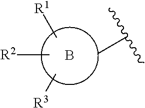

has a structure selected from:

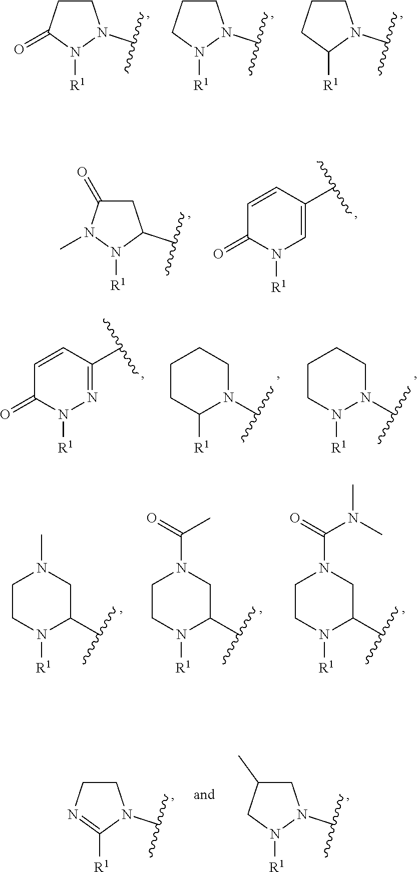

wherein [symbol] represents the point of attachment to L¹ in formula (I).

In some embodiments, the group

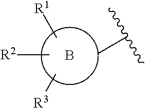

has a structure selected from:

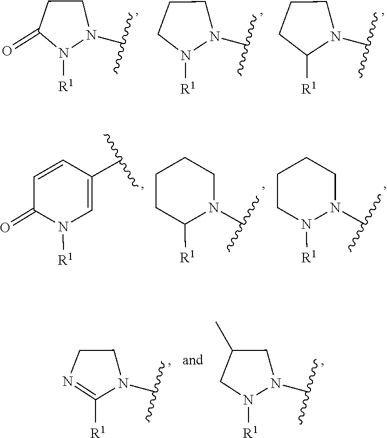

wherein [symbol] represents the point of attachment to L¹ in formula (I).

In some embodiments, the group

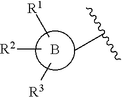

has a structure:

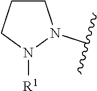

wherein [symbol] represents the point of attachment to L¹ in formula (I).

In some embodiments, R¹ is selected from aryl, heteroaryl, C₃₋₇ cycloalkyl, C₁₋₆ alkyl, C₃₋₇-cycloalkyl-C₁₋₃-alkyl, arylalkyl, heterocyclyl, and —C(O)heterocyclyl, each of which is independently unsubstituted or substituted with 1-3 substituents independently selected from halo, halo-C₁₋₃-alkyl, cyano, C₃₋₇ cycloalkyl, and pentafluorosulfanyl.

In some embodiments, R¹ is selected from:

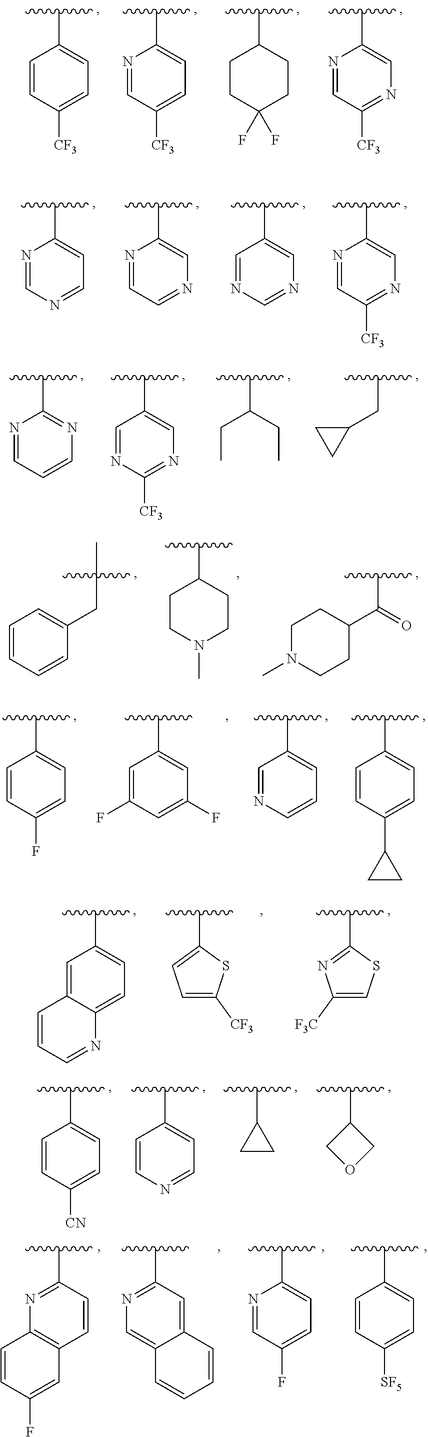

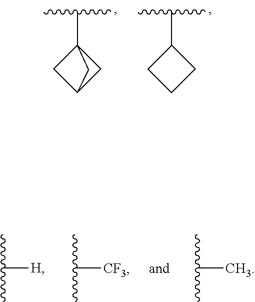

In some embodiments, R¹ is selected from:

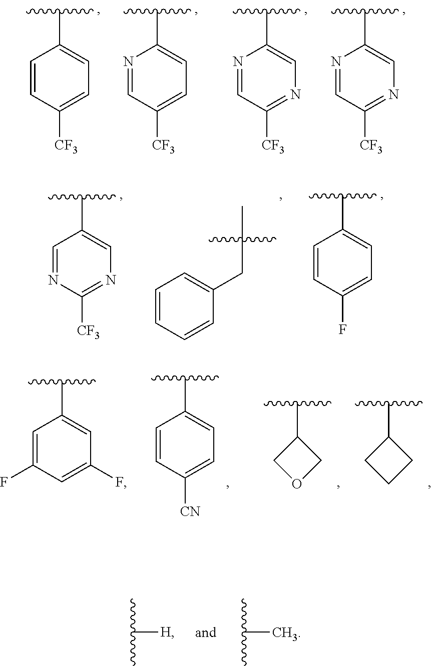

In some embodiments, the compound of formula (I) is a compound of formula (I′):

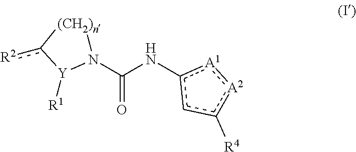

or a pharmaceutically acceptable salt thereof, wherein:
R¹ is selected from hydrogen, aryl, heteroaryl, and arylalkyl, wherein the aryl, heteroaryl, and heteroaryl are each independently unsubstituted or substituted with 1, 2, or 3 substituents independently selected from halo, halo-C₁₋₃-alkyl, and cyano;
R² is hydrogen or oxo;
n′ is 2 or 3;
Y is N or CH;
R⁴ is selected from C₁₋₆ alkyl, hydroxy-C₁₋₆ alkyl, C₃₋₇ cycloalkyl, and aryl, wherein aryl is unsubstituted or substituted with 1, 2, or 3 substituents independently selected from halo, halo-C₁₋₃-alkyl, and cyano; and
one of A¹ and A² is nitrogen and the other is oxygen; and
each dashed line represents the presence or absence of a bond.

In some embodiments, R¹ is selected from hydrogen, phenyl, benzyl, and a six-membered monocyclic heteroaryl having 1 or 2 nitrogen atoms, wherein the phenyl, benzyl, and heteroaryl are independently unsubstituted or substituted with 1 or 2 substituents independently selected from fluoro, trifluoromethyl, and cyano.

In some embodiments, R² is oxo. In some embodiments, R² is hydrogen.

In some embodiments, Y is N. In some embodiments, Y is CH.

In some embodiments, R⁴ is selected from tert-butyl, 1-hydroxy-2-methylpropan-2-yl, 2-hydroxypropan-2-yl, phenyl, cyclopropyl, and cyclohexyl, wherein the phenyl is optionally substituted with 1 fluoro.

In some embodiments, A¹ is oxygen and A² is nitrogen. In some embodiments, A¹ is nitrogen and A² is oxygen. In some embodiments, the group

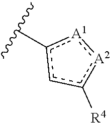

has a formula selected from:

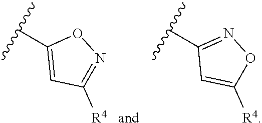

The present disclosure also provides a compound of formula (Ia):

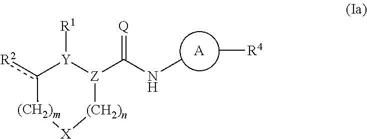

or a pharmaceutically acceptable salt thereof, wherein:
m and n are each independently 1, 2, 3, or 4;
X is a bond, O, NR⁵, or S(O)ₚ;
Y is N or CH;
p is 0, 1, or 2;
Z is CR³ or N;
Q is O, S, or NH;
A is a five- or six-membered heteroaryl having 1, 2, or 3 heteroatoms independently selected from N, O, and S;
the dashed line represents the presence or absence of a bond;
R¹, R², R³, R⁴, and R⁵ are each independently selected from hydrogen, C₁₋₆ alkyl, C₃₋₇ cycloalkyl, C₁₋₃-alkyl-C₃₋₇-cycloalkyl, C₃₋₇-cycloalkyl-C₁₋₃-alkyl, —C(O)—C₁₋₆ alkyl, aryl, and heteroaryl, each of which is unsubstituted or substituted with 1-6 substituents selected from halo, halo-C₁₋₃-alkyl, —OR^{6a}, and —(C₁₋₃ alkyl)-OR^{6b}; wherein, when the dashed line represents the presence of a bond, R² is oxo; and wherein R¹ and R² are optionally taken together with the atoms to which they are attached to form a heterocyclic ring; and
R^{6a} and R^{6b} are each independently selected from hydrogen, C₁₋₆ alkyl, and C₃₋₇ cycloalkyl.

In some embodiments, when R⁴ is unsubstituted alkyl, R¹ is not aryl, C₃₋₇ cycloalkyl, or C₁₋₃-alkyl-C₃₇-cycloalkyl. In some embodiments, when R⁴ is unsubstituted alkyl, m is 1 and n is 1. In some embodiments, R⁴ is substituted with at least one —OR⁶a group (e.g., at least one hydroxy group).

In some embodiments, X is a bond, O, NR⁵, S, or SO₂, and R⁵ is selected from hydrogen, methyl, ethyl, —C(O)CH₃, and phenyl. In some embodiments, X is a bond. In some embodiments, X is O. In some embodiments, X is NR⁵, wherein R⁵ is selected from hydrogen, C₁₋₆ alkyl (e.g., C₁₋₃ alkyl), —C(O)—C₁₋₆ alkyl, and aryl. In some embodiments, X is NR⁵, wherein R⁵ is selected from hydrogen, methyl, ethyl, —C(O)CH₃, and phenyl. In some embodiments, X is S(O)ₚ, and p is 0. In some embodiments, X is S(O)ₚ, and p is 2. In some embodiments, m and n are each independently selected from 1 and 2. In some embodiments, m is 1 and n is 2. In some embodiments, m is 1 and n is 1.

In some embodiments, Q is O or NH. In some embodiments, Q is O. In some embodiments, Q is NH. In some embodiments, Q is S.

In some embodiments, A is a five-membered heteroaryl having 1, 2, or 3 heteroatoms independently selected from N, O, and S, or a six-membered heteroaryl having 1 or 2 nitrogen atoms. In some embodiments, A is a five-membered heteroaryl having 1, 2, or 3 heteroatoms independently selected from N, O, and S. In some embodiments, A is a six-membered heteroaryl having 1 or 2 nitrogen atoms. In some embodiments, A is selected from isoxazole, pyrazole, thiadiazole (e.g., 1,2,4-thiadiazole or 1,3,4-thiadiazole), oxadiazole (e.g., 1,2,4-oxadiazole or 1,3,4-oxadiazole), pyridine, pyridazine, pyrimidine, and pyrazine. In some embodiments, A is selected from:

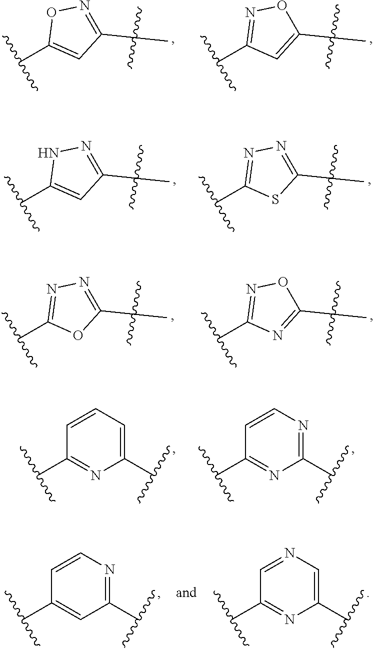

In some embodiments, R¹ is selected from C₁₋₆ alkyl, C₃₋₇-cycloalkyl-C₁₋₃-alkyl, aryl, and heteroaryl, each of which is unsubstituted or substituted with 1-3 substituents selected from halo and halo-C₁₋₃-alkyl. In some embodiments, R¹ is selected from methyl, isopropyl, cyclopropyl, cyclopropylmethyl, phenyl, and pyridyl, wherein the phenyl and pyridyl are unsubstituted or substituted with one substituent selected from halo and halo-C₁₋₃-alkyl. In some embodiments, R¹ is methyl. In some embodiments, R¹ is cyclopropyl. In some embodiments, R¹ is phenyl substituted with one trifluoromethyl group. In some embodiments, R¹ is pyridyl substituted with one trifluoromethyl group.

In some embodiments, the dashed line represents the absence of a bond, and R² is hydrogen. In some embodiments, the dashed line represents the presence of a bond, and R² is oxo.

In some embodiments, R¹ and R² are taken together with the atoms to which they are attached to form a six-membered heterocyclic ring.

In some embodiments, Y is N. In some embodiments, Y is CH.

In some embodiments, Z is CR³. In some embodiments, R³ is selected from hydrogen and C₁₋₆ alkyl. In some embodiments, R³ is selected from hydrogen and methyl. In some embodiments, Z is N.

In some embodiments, Y and Z are not simultaneously N. In some embodiments, when Y is N, Z is CR³. In some embodiments, when Z is N, Y is CH.

In some embodiments, R⁴ is selected from C₁₋₆ alkyl, C₃₋₇ cycloalkyl, and C₁₋₃-alkyl-C₃₋₇-cycloalkyl, each of which is unsubstituted or substituted with 1-4 substituents independently selected from halo, —OR^{6a}, and —(C₁₋₃ alkyl)-OR^{6b}. In some embodiments, R⁴ is C₁₋₆ alkyl substituted with 1-4 substituents independently selected from hydroxy and fluoro. In some embodiments, R⁴ is C₁₋₆ alkyl (e.g., ethyl, isopropyl, tert-butyl, isobutyl, or 3-methylbutan-2-yl), substituted with one hydroxy group. In some embodiments, R⁴ is C₃₋₇ cycloalkyl (e.g., cyclobutyl) substituted with one hydroxy group or one hydroxymethyl group. In some embodiments, R⁴ is C₁₋₃-alkyl-C₃₋₇-cycloalkyl (e.g., methylcyclopropyl) substituted with one hydroxy group. In some embodiments, R⁴ is unsubstituted C₁₋₆ alkyl.

In some embodiments, R⁴ is selected from:

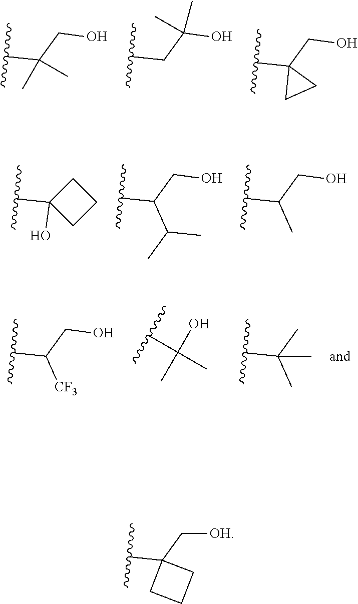

In some embodiments, the disclosure provides a compound selected from:

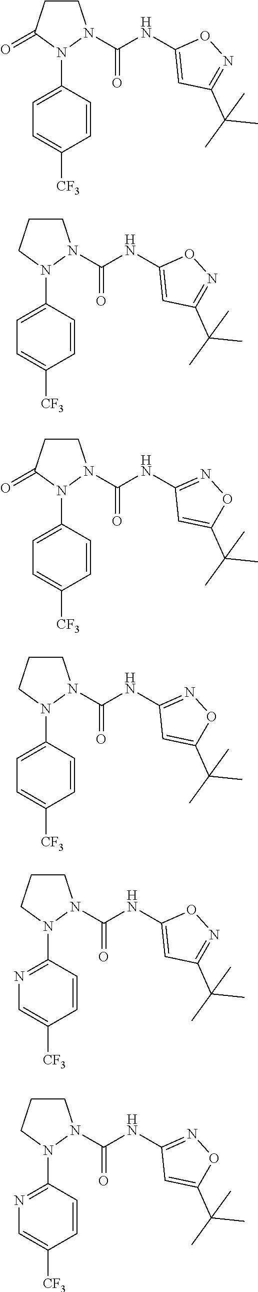

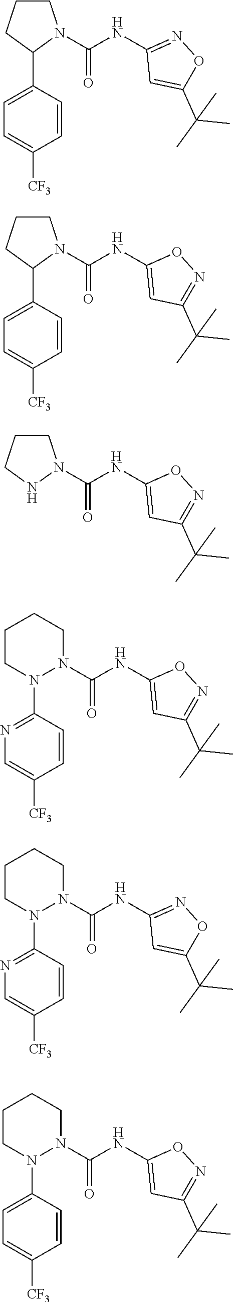

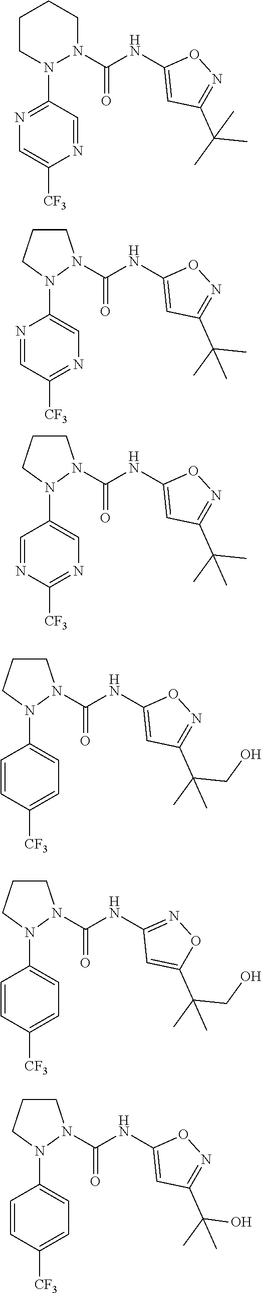

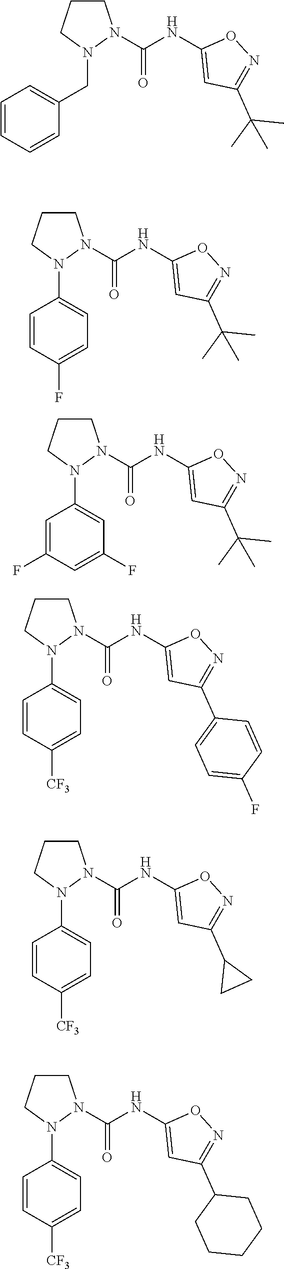

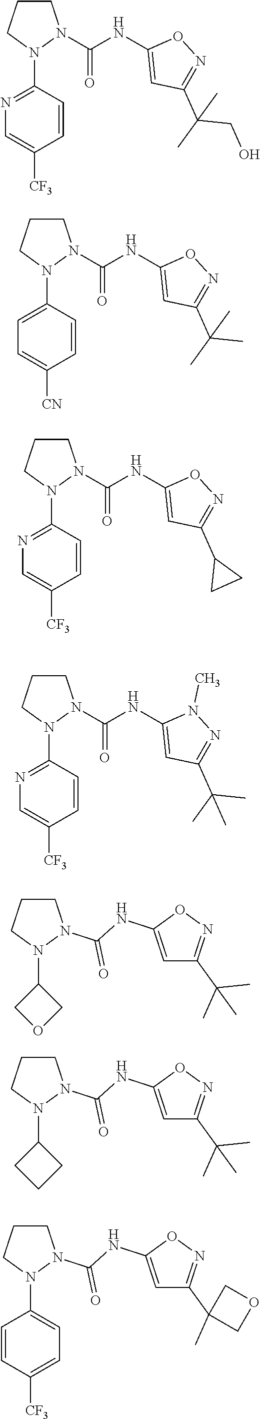

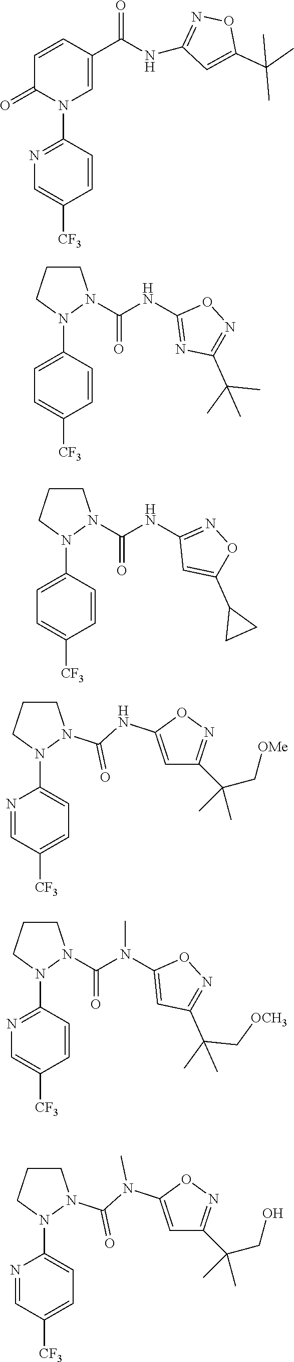

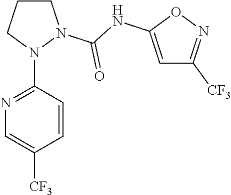 [SMILES: O=C(Nc1cc(C(F)(F)F)no1)N1CCCN1c1ccc(C(F)(F)F)cn1]

and pharmaceutically acceptable salts thereof.

Other exemplary compounds of formula (I) and/or formula (Ia) include the following:

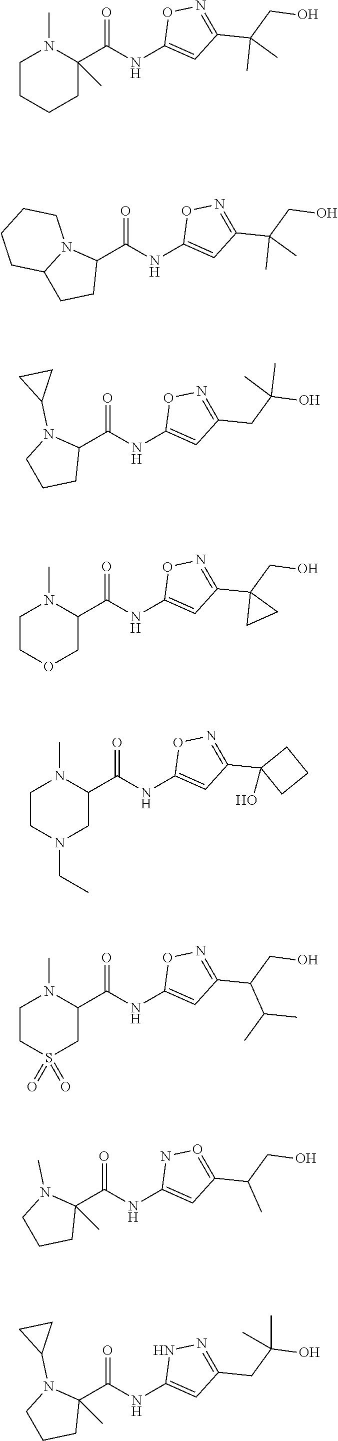

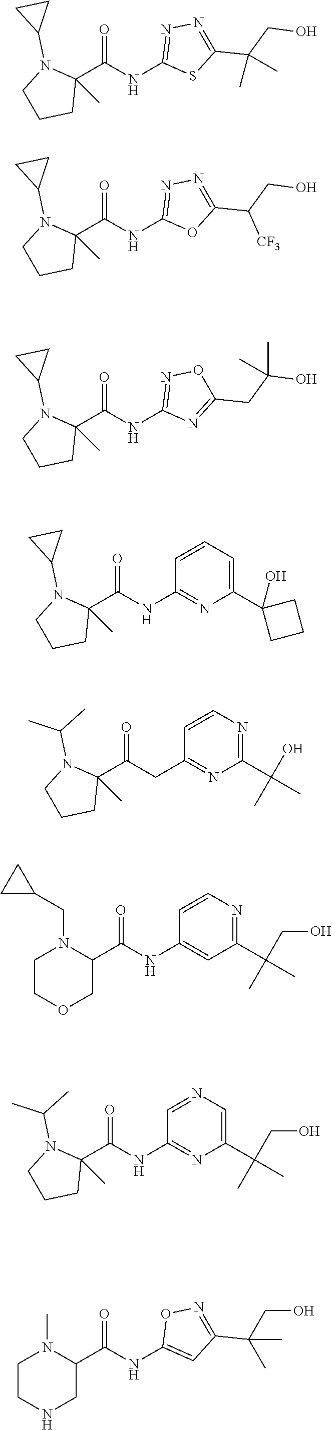

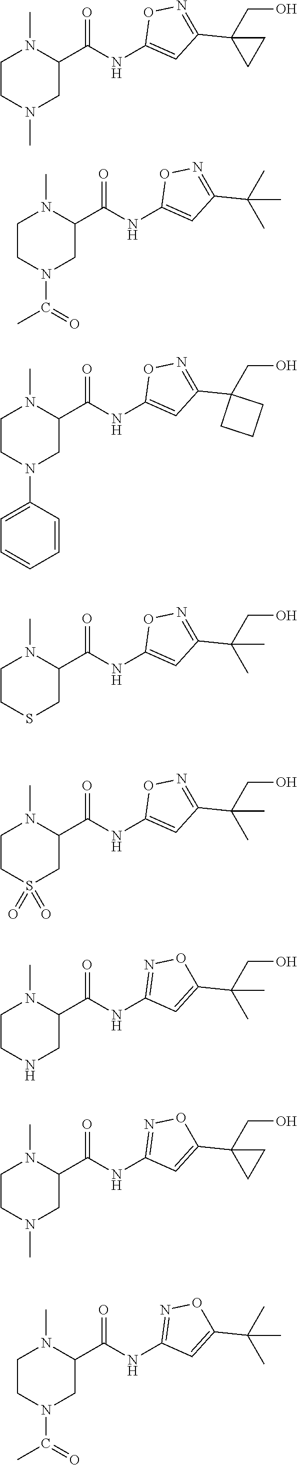

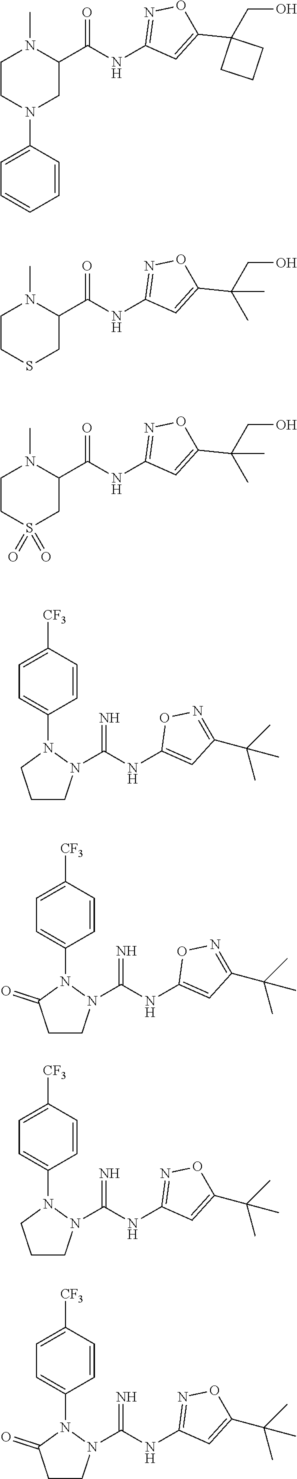

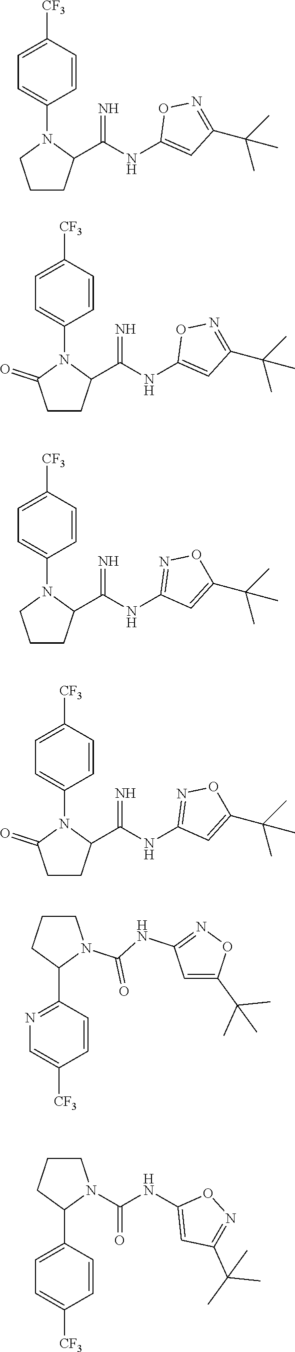

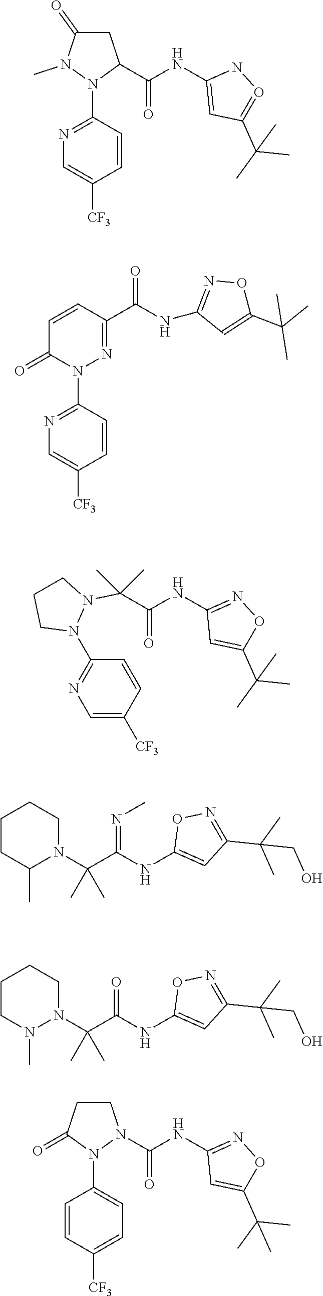

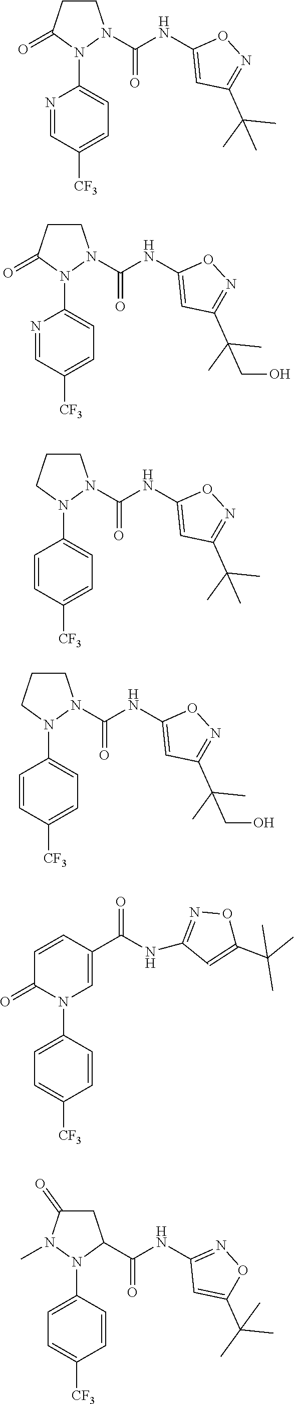

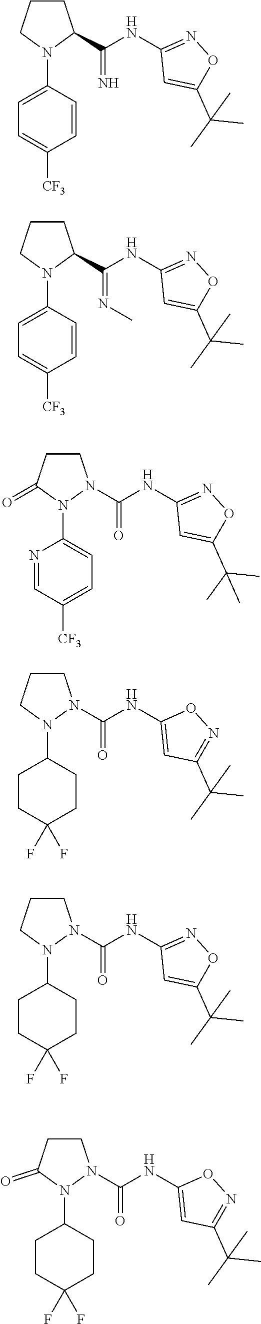

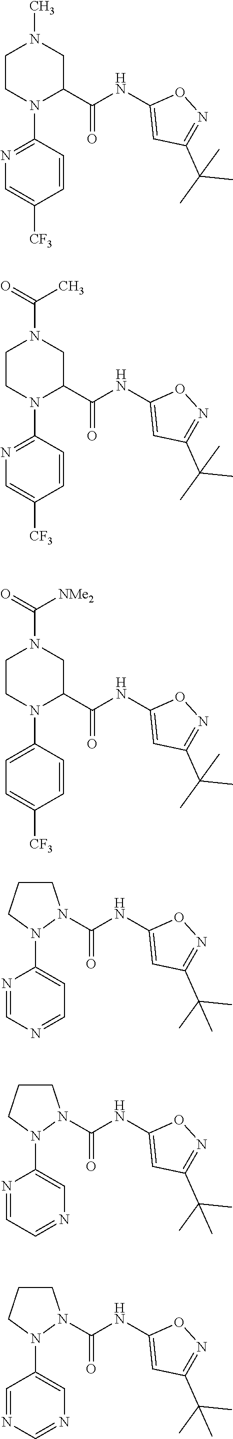

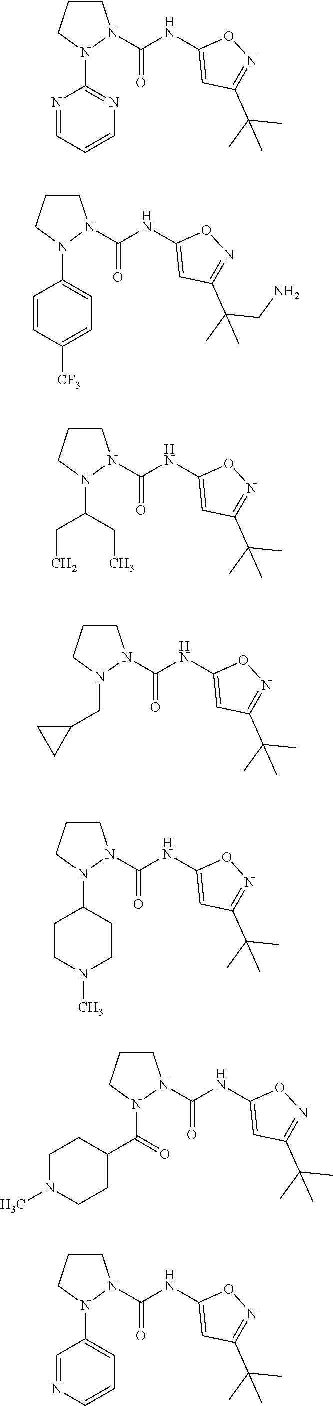

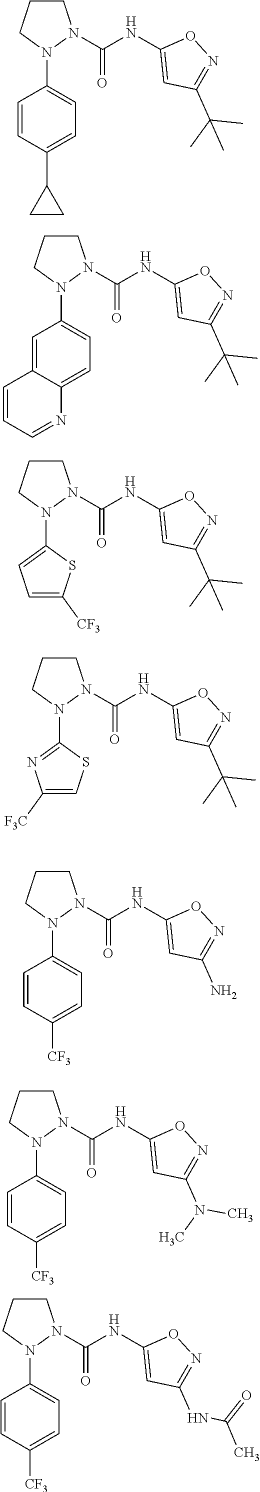

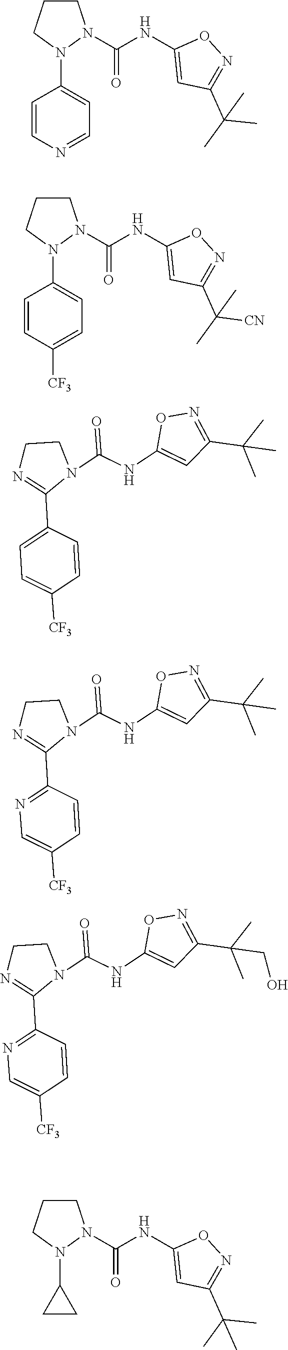

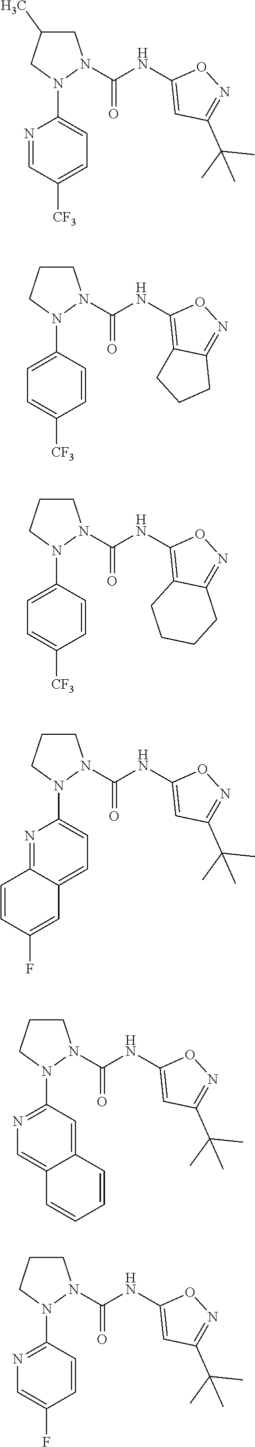

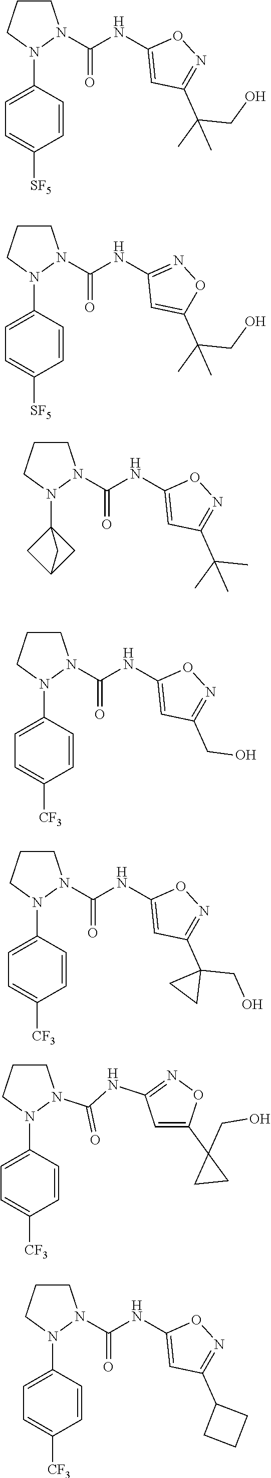

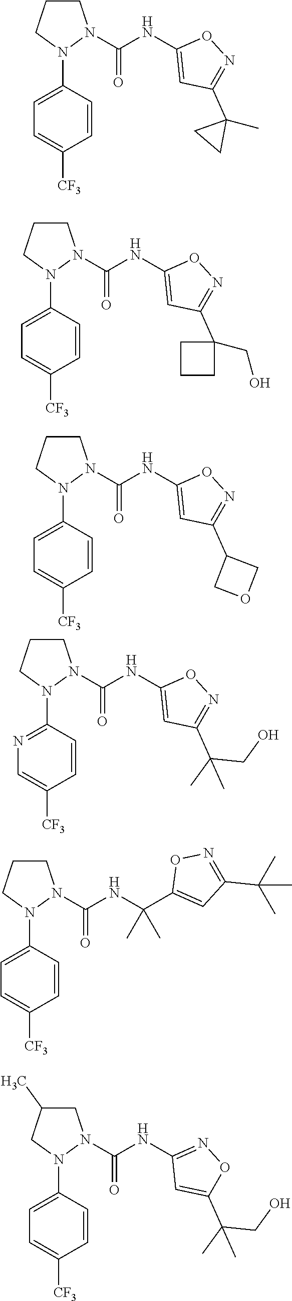

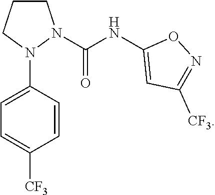 [SMILES: O=C(Nc1cc(C(F)(F)F)no1)N1CCCN1c1ccc(C(F)(F)F)cc1]

The present disclosure also provides a compound of formula (II):

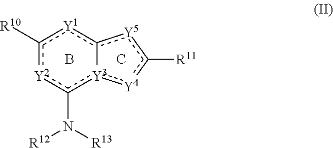

or a pharmaceutically acceptable salt thereof, wherein:
each dashed line represents the presence or absence of a bond, and each is independently chosen such that ring B and ring C are each heteroaryl;
Y¹ is selected from CR^{14a} and N;
Y² is selected from CR^{14b} and N;
Y³ is selected from C and N;
Y⁴ is selected from N, NR^{15a}, CR^{15b}, and S;
Y⁵ is selected from NR^{16a}, CR^{16b}, and S;
R¹⁰ is selected from hydrogen and C₁-C₄ alkyl;
R¹¹ is selected from heteroaryl and aryl, each of which is optionally substituted;
R¹² and R¹³ are each independently selected from hydrogen and C₁-C₆ alkyl, or R¹² and R¹³, together with the nitrogen atom to which they are attached, form an optionally substituted heterocyclic ring; and
R^{14a}, R^{14b}, R^{15s}, and R^{15b} are each independently selected from hydrogen and C₁-C₄ alkyl; and
R^{16a} and R^{16b} are each independently selected from hydrogen, C₁-C₆ alkyl, C₂-C₆ alkenyl, C₂-C₆ alkynyl, aryl, heteroaryl, cycloalkyl, and heterocyclyl, each of which is optionally substituted;
wherein Y¹, Y², Y⁴, and Y⁵ are not all simultaneously N.

In some embodiments, the compound has a structure selected from:

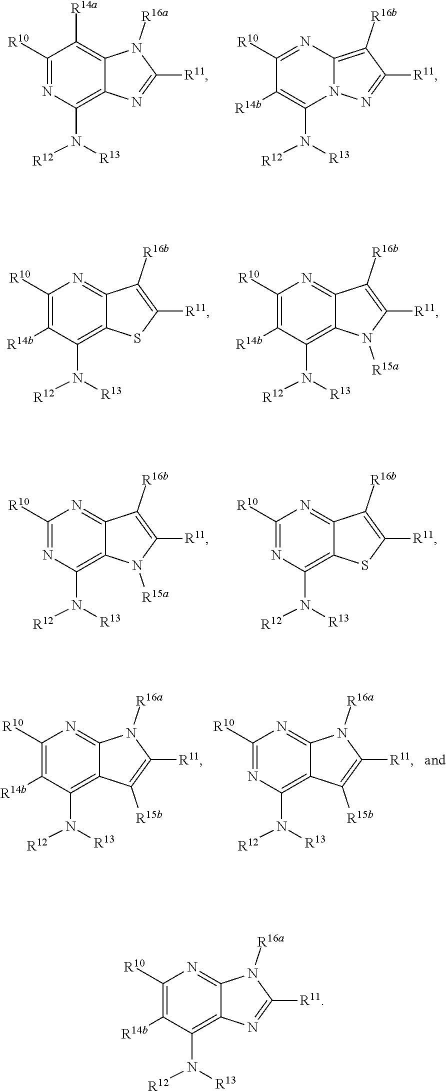

In some embodiments, Y¹ is CR^{14a}, and R^{14a} is hydrogen. In some embodiments, Y¹ is N.

In some embodiments, Y² is CR^{14b}, and R^{14b} is hydrogen. In some embodiments, Y² is N.

In some embodiments, Y³ is C. In some embodiments, Y³ is N.

In some embodiments, Y⁴ is N. In some embodiments, Y⁴ is NR¹⁵a, and R^{15a} is C₁-C₄ alkyl (e.g., methyl). In some embodiments, Y⁴ is CR¹⁵b, and R^{15b} is hydrogen (i.e. Y⁴ is CH). In some embodiments, Y⁴ is S.

In some embodiments, Y⁵ is NR^{16a}. In some embodiments, R^{16a} is heterocyclyl. In some embodiments, R^{16a} is monocyclic heterocyclyl. In some embodiments, R^{16a} is 5- or 6-membered monocyclic heterocyclyl having 1 or 2 heteroatoms independently selected from N, O, and S. In some embodiments, R^{16a} is a 6-membered monocyclic heterocyclyl having 1 heteroatom selected from N, O, and S. In some embodiments, R^{16a} is tetrahydropyranyl. In some embodiments, R^{16a} is tetrahydro-2H-pyran-4-yl. In some embodiments, Y⁵ is CR^{16b}, and R^{16b} is monocyclic heterocyclyl or aryl. In some embodiments, Y⁵ is CR^{16b}, and R^{16b} is monocyclic heterocyclyl. In some embodiments, R^{16b} is 5- or 6-membered monocyclic heterocyclyl having 1 or 2 heteroatoms independently selected from N, O, and S, or phenyl optionally substituted with one substituent (e.g., halo). In some embodiments, R^{16b} is 5- or 6-membered monocyclic heterocyclyl having 1 or 2 heteroatoms independently selected from N, O, and S. In some embodiments, R^{16b} is a 6-membered monocyclic heterocyclyl having 1 heteroatom selected from N, O, and S. In some embodiments, R^{16b} is tetrahydropyranyl. In some embodiments, R^{16b} is tetrahydro-2H-pyran-4-yl. In some embodiments, R^{16b} is phenyl substituted with one halo substituent.

In some embodiments, R¹⁰ is selected from hydrogen and methyl. In some embodiments, R¹⁰ is hydrogen. In some embodiments, R¹⁰ is C₁-C₄ alkyl In some embodiments, R¹⁰ is methyl.

In some embodiments, R¹¹ is aryl. In some embodiments, R¹¹ is phenyl substituted with 1 or 2 substituents independently selected from halo, C₁-C₄ alkyl, and C₁-C₄ alkoxy. In some embodiments, R¹¹ is phenyl substituted with 1 halo substituent. In some embodiments, R¹¹ is phenyl substituted with chloro. In some embodiments, R¹¹ is 2-chlorophenyl.

In some embodiments, R¹² and R¹³, together with the nitrogen atom to which they are attached, form a monocyclic 5-6 membered heterocyclic ring optionally substituted with 1 or 2 substituents independently selected from C₁-C₄ alkyl, halo, and C₁-C₄ alkoxy. In some embodiments, R¹² and R¹³, together with the nitrogen atom to which they are attached, form a 6-membered heterocyclic ring having 1 or 2 nitrogen atoms, wherein the heterocyclic ring is substituted with 1 substituent independently selected from C₁-C₄ alkyl, halo, and C₁-C₄ alkoxy. In some embodiments, R¹² and R¹³, together with the nitrogen atom to which they are attached, form a 6-membered heterocyclic ring having 2 nitrogen atoms, wherein the heterocyclic ring is substituted with 1 C₁-C₄ alkyl substituent. In some embodiments, R¹² and R¹³, together with the nitrogen atom to which they are attached, form a 4-methylpiperazin-1-yl ring.

In some embodiments, the compound of formula (II) is selected from:

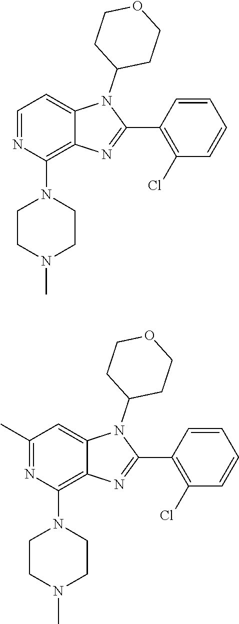 [SMILES: CN1CCN(c2nccc3c2nc(-c2ccccc2Cl)n3C2CCOCC2)CC1 ; Cc1cc2c(nc(-c3ccccc3Cl)n2C2CCOCC2)c(N2CCN(C)CC2)n1]

and pharmaceutically acceptable salts thereof.

Other exemplary compounds of formula (II) include the following:

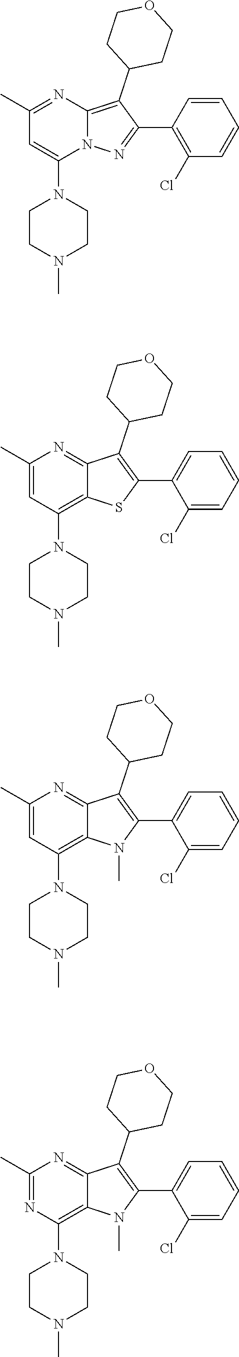 [SMILES: Cc1cc(N2CCN(C)CC2)n2nc(-c3ccccc3Cl)c(C3CCOCC3)c2n1 ; Cc1cc(N2CCN(C)CC2)c2sc(-c3ccccc3Cl)c(C3CCOCC3)c2n1 ; Cc1cc(N2CCN(C)CC2)c2c(n1)c(C1CCOCC1)c(-c1ccccc1Cl)n2C ; Cc1nc(N2CCN(C)CC2)c2c(n1)c(C1CCOCC1)c(-c1ccccc1Cl)n2C]

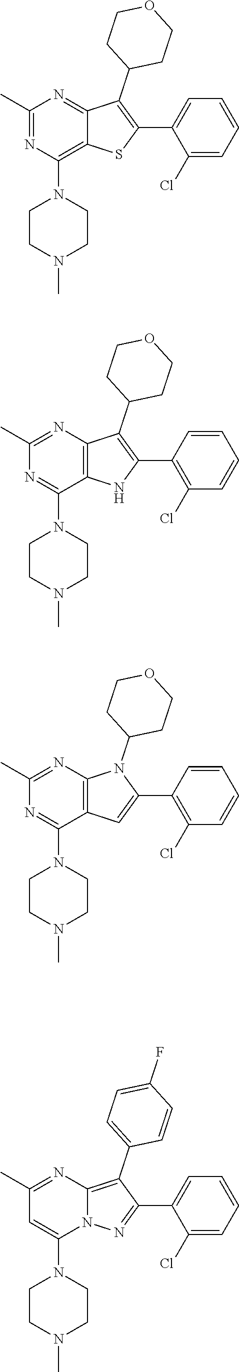 [SMILES: Cc1nc(N2CCN(C)CC2)c2sc(-c3ccccc3Cl)c(C3CCOCC3)c2n1 ; Cc1nc(N2CCN(C)CC2)c2[nH]c(-c3ccccc3Cl)c(C3CCOCC3)c2n1 ; Cc1nc(N2CCN(C)CC2)c2cc(-c3ccccc3Cl)n(C3CCOCC3)c2n1 ; Cc1cc(N2CCN(C)CC2)n2nc(-c3ccccc3Cl)c(-c3ccc(F)cc3)c2n1]

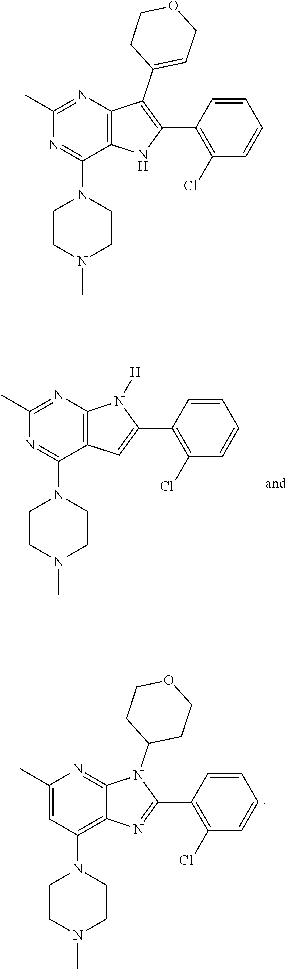 [SMILES: Cc1nc(N2CCN(C)CC2)c2[nH]c(-c3ccccc3Cl)c(C3=CCOCC3)c2n1 ; Cc1nc(N2CCN(C)CC2)c2cc(-c3ccccc3Cl)[nH]c2n1 ; Cc1cc(N2CCN(C)CC2)c2nc(-c3ccccc3Cl)n(C3CCOCC3)c2n1]

The compound (e.g., a compound of formula (I) or a compound of formula (II)) may exist as a stereoisomer wherein asymmetric or chiral centers are present. The stereoisomer is “R” or “S” depending on the configuration of substituents around the chiral carbon atom. The terms “R” and “S” used herein are configurations as defined in IUPAC 1974 Recommendations for Section E, Fundamental Stereochemistry, in Pure Appl. Chem., 1976, 45: 13-30. The disclosure contemplates various stereoisomers and mixtures thereof and these are specifically included within the scope of this invention. Stereoisomers include enantiomers and diastereomers, and mixtures of enantiomers or diastereomers. Individual stereoisomers of the compounds may be prepared synthetically from commercially available starting materials, which contain asymmetric or chiral centers or by preparation of racemic mixtures followed by methods of resolution well-known to those of ordinary skill in the art. These methods of resolution are exemplified by (1) attachment of a mixture of enantiomers to a chiral auxiliary, separation of the resulting mixture of diastereomers by recrystallization or chromatography and optional liberation of the optically pure product from the auxiliary as described in Furniss, Hannaford, Smith, and Tatchell, “Vogel's Textbook of Practical Organic Chemistry,” 5th edition (1989), Longman Scientific & Technical, Essex CM20 2JE, England, or (2) direct separation of the mixture of optical enantiomers on chiral chromatographic columns, or (3) fractional recrystallization methods.

The compound (e.g., a compound of formula (I) or a compound of formula (II)) may possess tautomeric forms, and tautomers also constitute embodiments of the disclosure.

The present disclosure also includes isotopically-labeled compound (e.g., an isotopically-labeled compound of formula (I) or an isotopically-labeled compound of formula (II)), which are identical to those recited in formula (I) or formula (II), but for the fact that one or more atoms are replaced by an atom having an atomic mass or mass number different from the atomic mass or mass number usually found in nature. Examples of isotopes suitable for inclusion in the compounds of the invention are hydrogen, carbon, nitrogen, oxygen, phosphorus, sulfur, fluorine, and chlorine, such as, but not limited to ²H, ³H, ¹³C, ¹⁴C, ¹⁵N, ¹⁸O, ³¹P, ³S, ¹⁸F, and ³⁶Cl, respectively. Substitution with heavier isotopes such as deuterium, i.e. ²H, can afford certain therapeutic advantages resulting from greater metabolic stability, for example increased in vivo half-life or reduced dosage requirements and, hence, may be preferred in some circumstances. The compound may incorporate positron-emitting isotopes for medical imaging and positron-emitting tomography (PET) studies for determining the distribution of receptors. Suitable positron-emitting isotopes that can be incorporated in compounds of formula (I) and formula (II) are ¹¹C, ¹³N, ¹⁵O, and ¹⁸F. Isotopically-labeled compounds of formula (I) and formula (II) can generally be prepared by conventional techniques known to those skilled in the art or by processes analogous to those described herein using an appropriate isotopically-labeled reagent in place of a non-isotopically-labeled reagent.

Certain compounds of formula (I) can be synthesized as illustrated in Scheme 1.

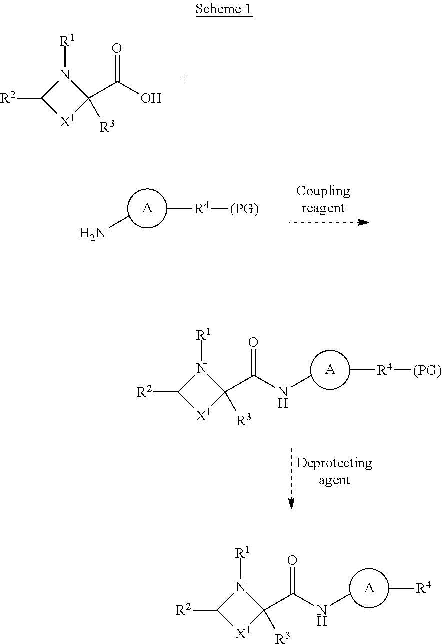

In Scheme 1, the group PG is an optional protecting group. The coupling reagent can be a standard reagent used for amide bond formation such as HATU, DCC, DIC/HOBT, BOP, PyBOP, or the like. The deprotecting agent will depend on the choice of the particular protecting group and the functional group being protected. In some embodiments, such as those in which R⁴ includes a hydroxy group, the protecting group is a tetrahydropyranyl ether, and the deprotecting agent is a protic acid such as hydrochloric acid or trifluoroacetic acid.

Certain compounds of formula (II) can be synthesized as illustrated in Scheme 2.

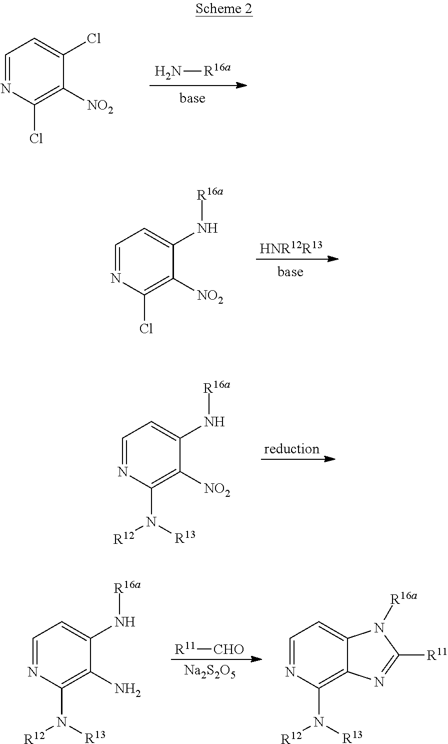

The compounds and intermediates may be isolated and purified by methods well-known to those skilled in the art of organic synthesis. Examples of conventional methods for isolating and purifying compounds can include, but are not limited to, chromatography on solid supports such as silica gel, alumina, or silica derivatized with alkylsilane groups, by recrystallization at high or low temperature with an optional pretreatment with activated carbon, thin-layer chromatography, distillation at various pressures, sublimation under vacuum, and trituration, as described for instance in “Vogel's Textbook of Practical Organic Chemistry,” 5th edition (1989), by Furniss, Hannaford, Smith, and Tatchell, pub. Longman Scientific & Technical, Essex CM20 2JE, England.

Reaction conditions and reaction times for each individual step can vary depending on the particular reactants employed and substituents present in the reactants used. Reactions can be worked up in a conventional manner, e.g., by eliminating the solvent from the residue and further purified according to methodologies generally known in the art such as, but not limited to, crystallization, distillation, extraction, trituration and chromatography. Unless otherwise described, the starting materials and reagents are either commercially available or can be prepared by one skilled in the art from commercially available materials using methods described in the chemical literature.

Standard experimentation, including appropriate manipulation of the reaction conditions, reagents and sequence of the synthetic route, protection of any chemical functionality that cannot be compatible with the reaction conditions, and deprotection at a suitable point in the reaction sequence of the method are included in the scope of the invention. Suitable protecting groups and the methods for protecting and deprotecting different substituents using such suitable protecting groups are well known to those skilled in the art; examples of which can be found in PGM Wuts and TW Greene, in Greene's book titled Protective Groups in Organic Synthesis (4^{th} ed.), John Wiley & Sons, NY (2006).

When an optically active form of a disclosed compound is required, it can be obtained by carrying out one of the procedures described herein using an optically active starting material (prepared, for example, by asymmetric induction of a suitable reaction step), or by resolution of a mixture of the stereoisomers of the compound or intermediates using a standard procedure (such as chromatographic separation, recrystallization or enzymatic resolution).

Similarly, when a pure geometric isomer of a compound is required, it can be obtained by carrying out one of the procedures described herein using a pure geometric isomer as a starting material, or by resolution of a mixture of the geometric isomers of the compound or intermediates using a standard procedure such as chromatographic separation.

The synthetic schemes and specific examples as described are illustrative and are not to be read as limiting the scope of the invention as it is defined in the claims. All alternatives, modifications, and equivalents of the synthetic methods and specific examples are included within the scope of the claims.

The disclosed compounds may exist as pharmaceutically acceptable salts. The term “pharmaceutically acceptable salt” refers to salts or zwitterions of the compounds which are water or oil-soluble or dispersible, suitable for treatment of disorders without undue toxicity, irritation, and allergic response, commensurate with a reasonable benefit/risk ratio and effective for their intended use. The salts may be prepared during the final isolation and purification of the compounds or separately by reacting an amino group of the compounds with a suitable acid. For example, a compound may be dissolved in a suitable solvent, such as but not limited to methanol and water and treated with at least one equivalent of an acid, like hydrochloric acid. The resulting salt may precipitate out and be isolated by filtration and dried under reduced pressure. Alternatively, the solvent and excess acid may be removed under reduced pressure to provide a salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetate, trifluoroacetate, glutamate, para-toluenesulfonate, undecanoate, hydrochloric, hydrobromic, sulfuric, phosphoric and the like. The amino groups of the compounds may also be quaternized with alkyl chlorides, bromides and iodides such as methyl, ethyl, propyl, isopropyl, butyl, lauryl, myristyl, stearyl, and the like.

Basic addition salts may be prepared during the final isolation and purification of the disclosed compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts can be prepared, such as those derived from methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine and N,N′-dibenzylethylenediamine, ethylenediamine, ethanolamine, diethanolamine, piperidine, piperazine, and the like.

Pharmaceutical Compositions

The disclosed compounds may be incorporated into pharmaceutical compositions suitable for administration to a subject (such as a patient, which may be a human or non-human). The pharmaceutical compositions may include a “therapeutically effective amount” or a “prophylactically effective amount” of the agent. A “therapeutically effective amount” refers to an amount effective, at dosages and for periods of time necessary, to achieve the desired therapeutic result. A therapeutically effective amount of the composition may be determined by a person skilled in the art and may vary according to factors such as the disease state, age, sex, and weight of the individual, and the ability of the composition to elicit a desired response in the individual. A therapeutically effective amount is also one in which any toxic or detrimental effects of a compound of the invention (e.g., a compound of formula (I) or a compound of formula (II)) are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease or condition, the prophylactically effective amount will be less than the therapeutically effective amount.

For example, a therapeutically effective amount of a compound of formula (I) or a compound of formula (II) may be about 1 mg/kg to about 1000 mg/kg, about 5 mg/kg to about 950 mg/kg, about 10 mg/kg to about 900 mg/kg, about 15 mg/kg to about 850 mg/kg, about 20 mg/kg to about 800 mg/kg, about 25 mg/kg to about 750 mg/kg, about 30 mg/kg to about 700 mg/kg, about 35 mg/kg to about 650 mg/kg, about 40 mg/kg to about 600 mg/kg, about 45 mg/kg to about 550 mg/kg, about 50 mg/kg to about 500 mg/kg, about 55 mg/kg to about 450 mg/kg, about 60 mg/kg to about 400 mg/kg, about 65 mg/kg to about 350 mg/kg, about 70 mg/kg to about 300 mg/kg, about 75 mg/kg to about 250 mg/kg, about 80 mg/kg to about 200 mg/kg, about 85 mg/kg to about 150 mg/kg, and about 90 mg/kg to about 100 mg/kg.

The pharmaceutical compositions may include pharmaceutically acceptable carriers. The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Thus, the compounds and their pharmaceutically acceptable salts may be formulated for administration by, for example, solid dosing, eye drop, in a topical oil-based formulation, injection, inhalation (either through the mouth or the nose), implants, or oral, buccal, parenteral, or rectal administration. Techniques and formulations may generally be found in “Remington's Pharmaceutical Sciences,” (Meade Publishing Co., Easton, Pa.). Therapeutic compositions must typically be sterile and stable under the conditions of manufacture and storage.

The route by which the disclosed compounds are administered and the form of the composition will dictate the type of carrier to be used. The composition may be in a variety of forms, suitable, for example, for systemic administration (e.g., oral, rectal, nasal, sublingual, buccal, implants, or parenteral) or topical administration (e.g., dermal, pulmonary, nasal, aural, ocular, liposome delivery systems, or iontophoresis).

Carriers for systemic administration typically include at least one of diluents, lubricants, binders, disintegrants, colorants, flavors, sweeteners, antioxidants, preservatives, glidants, solvents, suspending agents, wetting agents, surfactants, combinations thereof, and others. All carriers are optional in the compositions.

Suitable diluents include sugars such as glucose, lactose, dextrose, and sucrose; diols such as propylene glycol; calcium carbonate; sodium carbonate; sugar alcohols, such as glycerin; mannitol; and sorbitol. The amount of diluent(s) in a systemic or topical composition is typically about 50 to about 90%.

Suitable lubricants include silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil and oil of theobroma. The amount of lubricant(s) in a systemic or topical composition is typically about 5 to about 10%.

Suitable binders include polyvinyl pyrrolidone; magnesium aluminum silicate; starches such as corn starch and potato starch; gelatin; tragacanth; and cellulose and its derivatives, such as sodium carboxymethylcellulose, ethyl cellulose, methylcellulose, microcrystalline cellulose, and sodium carboxymethylcellulose. The amount of binder(s) in a systemic composition is typically about 5 to about 50%.

Suitable disintegrants include agar, alginic acid and the sodium salt thereof, effervescent mixtures, croscarmellose, crospovidone, sodium carboxymethyl starch, sodium starch glycolate, clays, and ion exchange resins. The amount of disintegrant(s) in a systemic or topical composition is typically about 0.1 to about 10%.

Suitable colorants include a colorant such as an FD&C dye. When used, the amount of colorant in a systemic or topical composition is typically about 0.005 to about 0.1%.

Suitable flavors include menthol, peppermint, and fruit flavors. The amount of flavor(s), when used, in a systemic or topical composition is typically about 0.1 to about 1.0%.

Suitable sweeteners include aspartame and saccharin. The amount of sweetener(s) in a systemic or topical composition is typically about 0.001 to about 1%.

Suitable antioxidants include butylated hydroxyanisole (“BHA”), butylated hydroxytoluene (“BHT”), and vitamin E. The amount of antioxidant(s) in a systemic or topical composition is typically about 0.1 to about 5%.

Suitable preservatives include benzalkonium chloride, methyl paraben and sodium benzoate. The amount of preservative(s) in a systemic or topical composition is typically about 0.01 to about 5%.

Suitable glidants include silicon dioxide. The amount of glidant(s) in a systemic or topical composition is typically about 1 to about 5%.

Suitable solvents include water, isotonic saline, ethyl oleate, glycerin, hydroxylated castor oils, alcohols such as ethanol, and phosphate buffer solutions. The amount of solvent(s) in a systemic or topical composition is typically from about 0 to about 100%.

Suitable suspending agents include AVICEL RC-591 (from FMC Corporation of Philadelphia, PA) and sodium alginate. The amount of suspending agent(s) in a systemic or topical composition is typically about 1 to about 8%.

Suitable surfactants include lecithin, Polysorbate 80, and sodium lauryl sulfate, and the TWEENS from Atlas Powder Company of Wilmington, Delaware. Suitable surfactants include those disclosed in the C.T.F.A. Cosmetic Ingredient Handbook, 1992, pp. 587-592; Remington's Pharmaceutical Sciences, 15th Ed. 1975, pp. 335-337; and McCutcheon's Volume 1, Emulsifiers & Detergents, 1994, North American Edition, pp. 236-239. The amount of surfactant(s) in the systemic or topical composition is typically about 0.1% to about 5%.

Although the amounts of components in the systemic compositions may vary depending on the type of systemic composition prepared, in general, systemic compositions include 0.01% to 50% of an active compound (e.g., a compound of formula (I) or a compound of formula (II)) or a compound of formula (II), and 50% to 99.99% of one or more carriers. Compositions for parenteral administration typically include 0.1% to 10% of actives and 90% to 99.9% of a carrier including a diluent and a solvent.

Compositions for oral administration can have various dosage forms. For example, solid forms include tablets, capsules, granules, and bulk powders. These oral dosage forms include a safe and effective amount, usually at least about 5%, and more particularly from about 25% to about 50% of actives. The oral dosage compositions include about 50% to about 95% of carriers, and more particularly, from about 50% to about 75%.

Tablets can be compressed, tablet triturates, enteric-coated, sugar-coated, film-coated, or multiple-compressed. Tablets typically include an active component, and a carrier comprising ingredients selected from diluents, lubricants, binders, disintegrants, colorants, flavors, sweeteners, glidants, and combinations thereof. Specific diluents include calcium carbonate, sodium carbonate, mannitol, lactose and cellulose. Specific binders include starch, gelatin, and sucrose. Specific disintegrants include alginic acid and croscarmellose. Specific lubricants include magnesium stearate, stearic acid, and talc. Specific colorants are the FD&C dyes, which can be added for appearance. Chewable tablets preferably contain sweeteners such as aspartame and saccharin, or flavors such as menthol, peppermint, fruit flavors, or a combination thereof.

Capsules (including implants, time release and sustained release formulations) typically include an active compound (e.g., a compound of formula (I) or a compound of formula (II)), and a carrier including one or more diluents disclosed above in a capsule comprising gelatin. Granules typically comprise a disclosed compound, and preferably glidants such as silicon dioxide to improve flow characteristics. Implants can be of the biodegradable or the non-biodegradable type.

The selection of ingredients in the carrier for oral compositions depends on secondary considerations like taste, cost, and shelf stability, which are not critical for the purposes of this invention.

Solid compositions may be coated by conventional methods, typically with pH or time-dependent coatings, such that a disclosed compound is released in the gastrointestinal tract in the vicinity of the desired application, or at various points and times to extend the desired action. The coatings typically include one or more components selected from the group consisting of cellulose acetate phthalate, polyvinyl acetate phthalate, hydroxypropyl methyl cellulose phthalate, ethyl cellulose, EUDRAGIT coatings (available from Evonik Industries of Essen, Germany), waxes and shellac.

Compositions for oral administration can have liquid forms. For example, suitable liquid forms include aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like. Liquid orally administered compositions typically include a disclosed compound and a carrier, namely, a carrier selected from diluents, colorants, flavors, sweeteners, preservatives, solvents, suspending agents, and surfactants. Peroral liquid compositions preferably include one or more ingredients selected from colorants, flavors, and sweeteners.

Other compositions useful for attaining systemic delivery of the subject compounds include sublingual, buccal and nasal dosage forms. Such compositions typically include one or more of soluble filler substances such as diluents including sucrose, sorbitol and mannitol; and binders such as acacia, microcrystalline cellulose, carboxymethyl cellulose, and hydroxypropyl methylcellulose. Such compositions may further include lubricants, colorants, flavors, sweeteners, antioxidants, and glidants.

The disclosed compounds can be topically administered. Topical compositions that can be applied locally to the skin may be in any form including solids, solutions, oils, creams, ointments, gels, lotions, shampoos, leave-on and rinse-out hair conditioners, milks, cleansers, moisturizers, sprays, skin patches, and the like. Topical compositions include: a disclosed compound (e.g., a compound of formula (I) or a compound of formula (II), or a pharmaceutically acceptable salt thereof), and a carrier. The carrier of the topical composition preferably aids penetration of the compounds into the skin. The carrier may further include one or more optional components.

The amount of the carrier employed in conjunction with a disclosed compound is sufficient to provide a practical quantity of composition for administration per unit dose of the compound. Techniques and compositions for making dosage forms useful in the methods of this invention are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2nd Ed., (1976).

A carrier may include a single ingredient or a combination of two or more ingredients. In the topical compositions, the carrier includes a topical carrier. Suitable topical carriers include one or more ingredients selected from phosphate buffered saline, isotonic water, deionized water, monofunctional alcohols, symmetrical alcohols, aloe vera gel, allantoin, glycerin, vitamin A and E oils, mineral oil, propylene glycol, PPG-2 myristyl propionate, dimethyl isosorbide, castor oil, combinations thereof, and the like. More particularly, carriers for skin applications include propylene glycol, dimethyl isosorbide, and water, and even more particularly, phosphate buffered saline, isotonic water, deionized water, monofunctional alcohols, and symmetrical alcohols.

The carrier of a topical composition may further include one or more ingredients selected from emollients, propellants, solvents, humectants, thickeners, powders, fragrances, pigments, and preservatives, all of which are optional.

Suitable emollients include stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, di-n-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate, and combinations thereof. Specific emollients for skin include stearyl alcohol and polydimethylsiloxane. The amount of emollient(s) in a skin-based topical composition is typically about 5% to about 95%.

Suitable propellants include propane, butane, isobutane, dimethyl ether, carbon dioxide, nitrous oxide, and combinations thereof. The amount of propellant(s) in a topical composition is typically about 0% to about 95%.

Suitable solvents include water, ethyl alcohol, methylene chloride, isopropanol, castor oil, ethylene glycol monoethyl ether, diethylene glycol monobutyl ether, diethylene glycol monoethyl ether, dimethylsulfoxide, dimethyl formamide, tetrahydrofuran, and combinations thereof. Specific solvents include ethyl alcohol and homotopic alcohols. The amount of solvent(s) in a topical composition is typically about 0% to about 95%.

Suitable humectants include glycerin, sorbitol, sodium 2-pyrrolidone-5-carboxylate, soluble collagen, dibutyl phthalate, gelatin, and combinations thereof. Specific humectants include glycerin. The amount of humectant(s) in a topical composition is typically 0% to 95%.

The amount of thickener(s) in a topical composition is typically about 0% to about 95%.

Suitable powders include beta-cyclodextrins, hydroxypropyl cyclodextrins, chalk, talc, fullers earth, kaolin, starch, gums, colloidal silicon dioxide, sodium polyacrylate, tetra alkyl ammonium smectites, trialkyl aryl ammonium smectites, chemically-modified magnesium aluminum silicate, organically-modified montmorillonite clay, hydrated aluminum silicate, fumed silica, carboxyvinyl polymer, sodium carboxymethyl cellulose, ethylene glycol monostearate, and combinations thereof. The amount of powder(s) in a topical composition is typically 0% to 95%.

The amount of fragrance in a topical composition is typically about 0% to about 0.5%, particularly, about 0.001% to about 0.1%.

Suitable pH adjusting additives include HCl or NaOH in amounts sufficient to adjust the pH of a topical pharmaceutical composition.

Compound Activities

Compounds disclosed herein modulate the CB2 receptor. CB2R ligands can be classified generally as agonists, antagonists, and inverse agonists. Compounds disclosed herein include agonists of the CB2 receptor. In some embodiments, compounds disclosed herein are partial agonists of the CB2 receptor. In some embodiments, compounds disclosed herein exhibit selectivity for the CB2 receptor compared to the CB1 receptor.

Different CB2R ligands have the ability to activate or inhibit different signaling pathways, which is known as functional selectivity or biased agonism. For example, certain compounds such as CP-55940 are agonists of both the cyclase pathway (inhibiting adenylyl cyclase and decreasing cAMP production, which suppresses cAMP-dependent protein kinase A activity), and the arrestin signaling pathway (recruiting arrestins, leading to receptor desensitization and internalization). Other compounds are known to be biased for one pathway or the other; for example, THC is a cyclase-biased ligand, while the compound GW833972A is an arrestin-biased ligand. See, e.g., Dhopeshwarkar et al. J. Pharmacol. Exp. Ther. 2016, 358(2):342-51. In some embodiments, compounds disclosed herein exhibit biased agonist activity. In some embodiments, compounds disclosed herein are cyclase-biased ligands. In some embodiments, compounds disclosed herein are arrestin-biased ligands. There may be particular advantages associated with cyclase-biased ligands; for example, activation of the beta-arrestin pathway can lead to receptor desensitization, and this has been suggested as a possible reason for the failure and lack of efficacy of non-biased CB2R agonists in past clinical trials for pain (Dhopeshwarkar et al. J. Pharmacol. Exp. Ther. 2016; 358(2):342-51).

Methods of Use

Compounds disclosed herein are selective CB2 agonists, and accordingly find use in methods of treating a variety of disorders, including addiction, pain, inflammatory disorders and other disorders having an inflammatory component, a disease having a neuroinflammatory or neurodegenerative component, Parkinson's disease, and other disorders.

a. Addiction

Compounds disclosed herein can be used to treat addiction/addictive disorders, including, e.g., opioid addiction, nicotine addiction, alcohol addiction, food addiction, methamphetamine addiction, cocaine addiction, or other rewarding behaviors.

Currently, selective CB2R agonists are being investigated for the treatment of nicotine, cocaine, and morphine addiction, with promising results in preclinical animal models (Galaj et al. CNS Drugs 2019; 33(10):1001-30). Selective CB2R agonist compounds disclosed herein accordingly should find use in similar methods.

In one embodiment, disclosed herein is a method of treating opioid addiction. More than 2 million Americans are addicted to opioids, resulting in ˜130 overdose deaths per day. The economic burden of prescription opioid misuse alone in the US is estimated to be $78.5 billion/year, including the costs of healthcare, lost productivity, addiction treatment, and criminal justice. Heroin is one of the main culprits in this opioid crisis, with nearly 80% of heroin users having first misused prescription opioids. Although there currently are approved heroin addiction treatments available such as methadone, these drugs also target the opioid receptors. Consequently, these treatments require strict government regulation and have high abuse potential. Additionally, methadone treatment is expensive requires clinical monitoring for safety.

Presented in the examples are data in a rodent model of heroin addiction, showing a selective CB2R agonist mitigates addictive-like behavior in rats. A selective, potent CB2R agonist may be effective in treating heroin use disorder by reducing the increase in dopamine (DA) release associated with heroin intake, thereby reducing the heroin craving behavior.

Accordingly, in some embodiments, disclosed herein is a method of treating opioid addiction in a subject in need thereof, comprising administering to the subject a therapeutically effective amount of a compound disclosed herein (e.g., a compound of formula (I) or a compound of formula (II)) or a pharmaceutically acceptable salt thereof, or a pharmaceutical composition disclosed herein (e.g., a pharmaceutical composition comprising a compound of formula (I) or a compound of formula (II), or a pharmaceutically acceptable salt thereof). In some embodiments, the subject in need of treatment is a subject having an opioid addiction. For example, in some embodiments, the subject has an addiction to an opioid selected from heroin, codeine, hydrocodone, morphine, oxycodone, hydromorphone, oxymorphone, fentanyl, dilaudid, tapentadol, methadone, buprenorphine, meperidine, tramadol, levorphanol, cocaine, and methamphetamine, or a salt or an ester of any thereof. In some embodiments, the subject has an addiction to heroin.

Compounds disclosed herein can also be used to treat addiction/addictive disorders, such as methamphetamine use disorder (MUD). About 1 million Americans suffer from MUD and about another 1 million suffer from cocaine use disorder. Psychosocial treatment is currently the standard treatment for methamphetamine dependence, but effects may not be sustained after cessation of treatment or as effective for the severe disorder, and pharmacological treatment has the potential to overcome these shortcomings (Siefried et al. CNS Drugs 2020; 34(4):337-65). Methamphetamine and cocaine are dopamine transporter (DAT) inhibitors that prevent dopamine (DA) reuptake to the cells and also lead to further DA release from the neurons, resulting in elevated levels of synaptic DA in circuits involved in reward and addiction (Newman et al. Annu. Rev. Pharmacolog. 2021; 61:609-28)

b. Pain

CB2 agonists have been shown to inhibit pain. For example, CB2 selective agonists blunt the pain response induced by thermal or other stimuli (Malan et al. Pain (2001) 93:239-45; Nackley et al. Neuroscience (2003) 119:747-57). CB2 activation has also been demonstrated to inhibit neuropathic pain response (Ibrahim et al. Proc. Natl. Acad. Sci. USA (2003) 100:10529-33). Agonists targeting CB2 receptors have been proposed as therapies for the treatment or management of a range of painful conditions, including acute pain, chronic inflammatory pain, and neuropathic pain (Ehrhart et al. J. Neuroinflammation 2005, 2:29). Accordingly, compounds and composition disclosed herein can be used in methods of treating:

Acute pain, such as dental pain, perioperative, post-operative pain, traumatic pain, muscle pain, pain in burned skin, sun burn, trigeminal neuralgia, sun burn; spasm of the gastrointestinal tract or uterus, colics;

Visceral pain, such as pain associated with chronic pelvic pain, pancreatitis, peptic ulcer, interstitial cystitis, renal colic, angina, dysmenorrhea, menstruation, gynecological pain, irritable bowel syndrome (IBS), non-ulcer dyspepsia, non-cardiac chest pain, myocardial ischemia;

Neuropathic pain, such as low back pain, non-herpetic neuralgia, post herpetic neuralgia, diabetic neuropathy, nerve injury, acquired immune deficiency syndrome (AIDS) related neuropathic pain, head trauma, painful traumatic mononeuropathy, toxin and chemotherapy induced pain, phantom limb pain, painful polyneuropathy, thalamic pain syndrome, post-stroke pain, central nervous system injury, post surgical pain, stump pain, repetitive motion pain, pain induced by post mastectomy syndrome, multiple sclerosis, root avulsions, post-thoracotomy syndrome, neuropathic pain associated hyperalgesia and allodynia.

Inflammatory/nociceptive pain induced by or associated with disorders such as osteoarthritis, rheumatoid arthritis, rheumatic disease, tenosynovitis, gout, vulvodynia, myofascial pain (muscular injury, fibromyalgia), tendonitis, osteoarthritis, juvenile arthritis, spondylitis, gouty arthritis, psoriatic arthritis, musculoskeletal pain, fibromyalgia, sprains and strains, sympathetically maintained pain, myositis, pain associated with migraine, toothache, influenza and other viral infections such as the common cold, rheumatic fever, systemic lupus erythematosus; and

Cancer pain induced by or associated with tumors such as lymphatic leukemia, Hodgkin's disease, malignant lymphoma, lymphogranulomatosis, lymphosarcoma, solid malignant tumors, or extensive metastases.

c. Inflammation/Disorders Accompanied by an Inflammatory Process

CB2 selective ligands have been developed and tested for their effects in various inflammatory settings. For example, in animal models of inflammation, CB2 selective agonists, inverse agonists and antagonists have been shown to be effective in suppressing inflammation (Hanus et al. Proc. Natl. Acad. Sci. USA 1999, 96:14228-14233; Ueda et al. Eur. J. Pharmacol. 2005, 520:164-171; Smith et al. Eur. J. Pharmacol. 2001, 432: 107-119). Accordingly, some embodiments, the disclosure provides a method of treating inflammation in a subject in need thereof, comprising administering to the subject an effective amount of a compound disclosed herein (e.g., a compound of formula (I) or a compound of formula (II)) or a pharmaceutically acceptable salt thereof, or a pharmaceutical composition disclosed herein (e.g., a pharmaceutical composition comprising a compound of formula (I) or a compound of formula (II), or a pharmaceutically acceptable salt thereof). In some embodiments, the disclosure provides a method of treating a disorder that is accompanied by an inflammatory process in a subject in need thereof, comprising administering to the subject an effective amount of a compound disclosed herein (e.g., a compound of formula (I) or a compound of formula (II)) or a pharmaceutically acceptable salt thereof, or a pharmaceutical composition disclosed herein (e.g., a pharmaceutical composition comprising a compound of formula (I) or a compound of formula (II), or a pharmaceutically acceptable salt thereof). For example, disorders or indications that are accompanied by an inflammatory process include:

Lung diseases: e.g. asthma, bronchitis, allergic rhinitis, emphysema, adult respiratory distress syndrome (ARDS), pigeon fancier's disease, farmer's lung, chronic obstructive pulmonary disease (COPD), asthma including allergic asthma (atopic or non-atopic) as well as exercise-induced bronchoconstriction, occupational asthma, viral- or bacterial exacerbation of asthma, other non-allergic asthmas and “wheezy-infant syndrome,” pneumoconiosis, including aluminosis, anthracosis, asbestosis, chalicosis, ptilosis, siderosis, silicosis, tabacosis and byssinosis;

Rheumatic diseases or autoimmune diseases or musculoskeletal diseases: all forms of rheumatic diseases, especially rheumatoid arthritis, acute rheumatic fever, and polymyalgia rheumatica; reactive arthritis; rheumatic soft tissue diseases; inflammatory soft tissue diseases of other genesis; arthritic symptoms in degenerative joint diseases (arthroses); tendinitis, bursitis, osteoarthritis, traumatic arthritis; collagenoses of any genesis, e.g., systemic lupus erythematosus, scleroderma, polymyositis, dermatomyositis, Sjogren syndrome, Still disease, Felty syndrome; and osteoporosis and other bone resorption diseases;

Allergic diseases: all forms of allergic reactions, e.g., angioneurotic edema, hay fever, insect bites, allergic reactions to drugs, blood derivatives, contrast agents, etc., anaphylactic shock (anaphylaxis), urticaria, angioneurotic edema, and contact dermatitis;

Vascular diseases: polyarteritis nodosa, polyarteritis nodosa, periarteritis nodosa, arteritis temporalis, Wegner granulomatosis, giant cell arthritis, atherosclerosis, reperfusion injury and erythema nodosum;

Dermatological diseases: e.g. dermatitis, psoriasis; sunburn, burns, eczema; (vi) Renal diseases: e.g. nephrotic syndrome; and all types of nephritis, e.g., glomerulonephritis; pancreatitis;

Hepatic diseases: e.g. acute liver cell disintegration; acute hepatitis of various genesis, e.g., viral, toxic, drug-induced; and chronically aggressive and/or chronically intermittent hepatitis;

Gastrointestinal diseases: e.g. inflammatory bowel diseases, irritable bowel syndrome, regional enteritis (Crohns disease), colitis ulcerosa; gastritis; aphthous ulcer, celiac disease, regional ileitis, gastroesophageal reflux disease;

Neuroprotection: e.g. in the treatment of neurodegeneration following stroke; cardiac arrest; pulmonary bypass; traumatic brain injury; spinal cord injury or the like;

Eye diseases: allergic keratitis, uveitis, or iritis; conjunctivitis; blepharitis; neuritis nervi optici; choroiditis; glaucoma and sympathetic ophthalmia;

Diseases of the ear, nose, and throat (ENT) area: e.g. tinnitus; allergic rhinitis or hay fever; otitis externa; caused by contact eczema, infection, etc.; and otitis media;

Neurological diseases: e.g. brain edema, particularly tumor-related brain edema; multiple sclerosis; acute encephalomyelitis; meningitis; acute spinal cord injury; trauma; dementia, particularly degenerative dementia (including senile dementia, Alzheimer's disease; Parkinson's disease and Creutzfeldt-Jacob disease; Huntington's chorea, Pick's disease; motor neuron disease), vascular dementia (including multi-infarct dementia) as well as dementia associated with intracranial space occupying lesions; infections and related conditions (including HIV infection); Guillain-Barre syndrome; myasthenia gravis, stroke; and various forms of seizures, e.g., nodding spasms;

Blood diseases: acquired hemolytic anemia; aplastic anemia, and idiopathic thrombocytopenia;

Tumor diseases: acute lymphatic leukemia; Hodgkin's disease, malignant lymphoma; lymphogranulomatoses; lymphosarcoma; solid malignant tumors; extensive metastases;

Endocrine diseases: endocrine ophthalmopathy; endocrine orbitopathia; thyrotoxic crisis; Thyroiditis de Quervain; Hashimoto thyroiditis; Morbus Basedow; granulomatous thyroiditis; struma lymphomatosa; and Graves' disease; type I diabetes (insulin-dependent diabetes);

Organ and tissue transplantations and graft-versus-host diseases;

Severe states of shock, e.g., septic shock, anaphylactic shock, and systemic inflammatory response syndrome (SIRS);

Headache such as cluster headache, migraine with and without aura, tension type headache, headache with different origins, headache disorders including prophylactic and acute use; and

Various other disease-states or conditions including, restenosis following percutaneous transluminal coronary angioplasty, acute and chronic pain, atherosclerosis, reperfusion injury, congestive heart failure, myocardial infarction, thermal injury, multiple organ injury secondary to trauma, necrotizing enterocolitis and syndromes associated with hemodialysis, leukopheresis, and granulocyte transfusion, sarcoidosis, gingivitis, pyrexia, edema resulting from trauma associated with burns, sprains or fracture, cerebral oedema and angioedema, Diabetes such as diabetic vasculopathy, diabetic neuropathy, diabetic retinopathy, post capillary resistance or diabetic symptoms associated with insulitis (e.g. hyperglycemia, diuresis, proteinuria and increased nitrite and kallikrein urinary excretion).

d. Diseases Having a Neuroinflammatory or Neurodegenerative Component

CB2 receptor agonists have also been proposed for treating diseases that have a neuroinflammatory or neurodegenerative component, such as multiple sclerosis (Pertwee et al. Mol. Neurobiol. 2007, 36:45-59; Dittel, Br. J. Pharmacol. 2008, 153:271-276; Baker et al. Nature (2000) 404:84-87; Arevalo-Martin et al. J. Neurosci. (2003) 23:2511-2516), amyotrophic lateral sclerosis (Kim et al. Eur. J. Pharmacol. 2006, 542:100-105; Shoemaker et al. J. Neurochem. 2007, 101:87-98), Huntington's disease (Sagredo et al. Recent Patents CNS Drug Discov. 2012, 7:41-48), and stroke (Zhang et al. J. Cereb. Blood Flow Metab. 2007, 27:1387-1396; Pacher et al. Br. J. Pharmacol. 2008, 153:252-262). Accordingly, in some embodiments, the disclosure provides a method of treating a disease having a neuroinflammatory or neurodegenerative component in a subject in need thereof, comprising administering to the subject an effective amount of a compound disclosed herein (e.g., a compound of formula (I) or a compound of formula (II)) or a pharmaceutically acceptable salt thereof, or a pharmaceutical composition disclosed herein (e.g., a pharmaceutical composition comprising a compound of formula (I) or a compound of formula (II), or a pharmaceutically acceptable salt thereof). In some embodiments, the disease having a neuroinflammatory or neurodegenerative component is selected from multiple sclerosis, amyotrophic lateral sclerosis, Huntington's disease, and stroke.

e. Parkinson's Disease

Parkinson's disease (PD) is one of the world's fastest-growing neurological disorders. Evidence indicates that inflammation and the involvement of the acquired and innate immune systems are instrumental in leading neuronal death and disease progression in PD patients (Tan et al. Nat Rev Neurol. 2020; 16(6):303-18). Activated microglia, brain infiltration of T-lymphocytes, and impaired BBB are common denominators of PD pathophysiology (Alvarez-Luquin et al. J. Neuroinflamm. 2019; 16(1):1-11. The intertwining of innate and adaptive immunity is modulated by the ECS, especially through the CB2 receptor, which is abundantly expressed on microglia (Walter et al. J Neurosci. 2003; 23(4):1398-405). A few reports have shown increased levels of endocannabinoids in the CSF of PD patients (Pisani et al. Movement Disord. 2010; 25(7):920-4), while increased levels of CB2R have been observed in neurotoxin-based mouse models of PD (Price et al. Eur. J. Neurosci. 2009; 29(11):2177-86. Pharmacological targeting of CB2R could be a promising therapeutic strategy to curtail both symptoms and disease progression in PD patients. For instance, nabilone, which is a partial CB1 and CB2 agonist, alleviates non-motor symptoms in PD patients (Peball et al. Ann Neurol. 2020; 88(4):712-22). Furthermore, previous studies showed that selective pharmacological activation of CB2R using HU-308 alone or in conjunction with amantadine had a dual therapeutic effect on neuroinflammation and L-DOPA induced dyskinesia in a 6-hydroxydopamine (6-OHDA) model of PD (Rentsch et al. Neurobiol Dis. 2020; 134:104646).

Accordingly, in some embodiments, the disclosure provides a method of treating Parkinson's disease in a subject in need thereof, comprising administering to the subject an effective amount of a compound disclosed herein (e.g., a compound of formula (I) or a compound of formula (II)) or a pharmaceutically acceptable salt thereof, or a pharmaceutical composition disclosed herein (e.g., a pharmaceutical composition comprising a compound of formula (I) or a compound of formula (II), or a pharmaceutically acceptable salt thereof).

f. Other Disorders

CB2 receptor agonists have also been proposed for treating other disorders including emesis (see, e.g., Van Sickle et al. Science 2005, 310:329-332), therapeutics ischemia/reperfusion injury (see, e.g., Bátkai et al. FASEB J. 2007, 21:1788-1800), renal fibrosis (see, e.g., Barutta et al. Diabetes 2011, 60:2386-2396), liver diseases such as liver cirrhosis (see, e.g., Mallat et al. Expert Opin, Ther. Targets 2007, 11:43-409; Izzo et al. Gut 2008, 57:1140-1155; Lotersztajn et al. Br. J. Pharmacol. 2008, 153:286-289; Dai et al. Int. J. Infect. Dis. 2017, 59:124-130), cancer (see, e.g., Guzmán, Nat. Rev. Cancer 2003, 3:745-755; Izzo et al. Gut 2008, 57:1140-1155; Wright et al. Br. J. Pharmacol. 2008, 153:263-270), epilepsy, septic shock (e.g., as antihypovolemic and/or antihypotensive agents), benign prostatic hyperplasia and hyperactive bladder, pruritis, vitiligo, general gastrointestinal disorders, disturbances of visceral motility at respiratory, genitourinary, gastrointestinal or vascular regions, wounds, burns, tissue damage and postoperative fever, and syndromes associated with itching.

f. Selective Agonization of CB2R

Also disclosed herein is method of selectively agonizing a cannabinoid 2 receptor in a subject, comprising administering to the subject an effective amount of a compound disclosed herein (e.g., a compound of formula (I) or a compound of formula (II)) or a pharmaceutically acceptable salt thereof, or a pharmaceutical composition disclosed herein (e.g., a pharmaceutical composition comprising a compound of formula (I) or a compound of formula (II), or a pharmaceutically acceptable salt thereof).

f. Modulation of Dopamine Transmission

Compounds disclosed herein have the ability to modify and modulate dopaminergic signaling. Accordingly, the compounds can be used in a method of modulating dopaminergic signaling in a subject in need thereof, comprising administering to the subject an effective amount of a compound disclosed herein (e.g., a compound of formula (I) or a compound of formula (II)) or a pharmaceutically acceptable salt thereof, or a pharmaceutical composition disclosed herein (e.g., a pharmaceutical composition comprising a compound of formula (I) or a compound of formula (II), or a pharmaceutically acceptable salt thereof).

g. Dosages and Administration

It will be appreciated that appropriate dosages of the compounds, and compositions comprising the compounds, can vary from patient to patient. Determining the optimal dosage will generally involve the balancing of the level of therapeutic benefit against any risk or deleterious side effects of the treatments described herein. The selected dosage level will depend on a variety of factors including, but not limited to, the activity of the particular compound, the route of administration, the time of administration, the rate of excretion of the compound, the duration of the treatment, other drugs, compounds, and/or materials used in combination, and the age, sex, weight, condition, general health, and prior medical history of the patient. The amount of compound and route of administration will ultimately be at the discretion of the physician, although generally the dosage will be to achieve local concentrations at the site of action which achieve the desired effect without causing substantial harmful or deleterious side-effects.

Administration in vivo can be effected in one dose, continuously or intermittently (e.g. in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician.

A compound described herein may be used in combination with other known therapies. Administered “in combination,” as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

A compound described herein and the at least one additional therapeutic agent can be administered simultaneously, in the same or in separate compositions, or sequentially. For sequential administration, the compound described herein can be administered first, and the additional agent can be administered subsequently, or the order of administration can be reversed.

In some embodiments, a compound described herein is administered with at least one additional therapeutic agent, such as an anti-addiction agent. An anti-addiction agent may be an agent that helps with cravings, or an agent that aids with opioid withdrawal. Examples of anti-addiction agents that may be used as an additional active ingredient include, but are not limited to, buprenorphine, bupropion, buspirone, clonidine, levomethadyl acetate, lofexidine, methadone, naloxone, and naltrexone. In some embodiments, a compound described herein is administered in conjunction with another mode of therapy, such as a behavioral therapy or counseling.

In some embodiments, a compound or composition described herein is co-administered with a peripherally restricted cannabinoid receptor antagonist to block the peripheral effects while maintaining the central effects. In some embodiments, a compound or composition described herein is co-administered with a centrally active cannabinoid receptor antagonist to block the central effects while maintaining the peripheral effects.

In some embodiments, a compound or compound described herein is co-administered with a selective CB2R agonist. In some embodiments, the selective CB2R agonist is selected from PRS-211375, GW842166, GRC10693 (tedalinab), KHK6188, ABT521, LY 2828360, APD 371 (olorinab), or S-777469.

The following examples further illustrate aspects of the disclosure but, of course, should not be construed as in any way limiting its scope.

### EXAMPLES

Abbreviations used in the Examples include the following: ACN is acetonitrile; DCM is dichloromethane; DIPEA is N,N-diisopropylethylamine; DMSO is dimethylsulfoxide; EtOH is ethanol; LCMS is liquid chromatography mass spectrometry; MeOH is methanol; TEA is triethylamine; THF is tetrahydrofuran; and TLC is thin layer chromatography.

### Example 1: Compound Syntheses

### 2-(2-chlorophenyl)-4-(4-methylpiperazin-1-yl)-1-(tetrahydro-2H-pyran-4-yl)-1H-imidazo[4,5-c]pyridine (Compound 1)

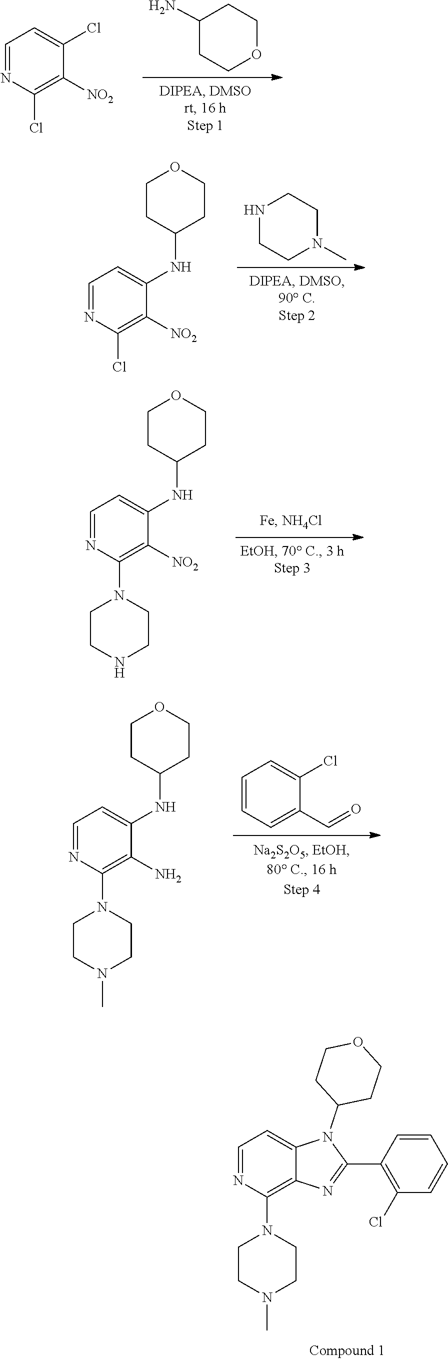

Step 1: 2-chloro-3-nitro-N-(tetrahydro-2H-pyran-4-yl)pyridin-4-amine. To a stirred solution of 2,4-dichloro-3-nitropyridine (1.5 g, 7.772 mmol), and tetrahydro-2H-pyran-4-amine (0.785 g, 7.772 mmol) in DMSO, was added DIPEA (2.03 mL, 11.658 mmol) and the resulting reaction mixture was allowed to stir at 25° C. for 16 h. The reaction mixture was diluted with ice cold water and the precipitated solid was filtered and dried under vacuo to afford 2-chloro-3-nitro-N-(tetrahydro-2H-pyran-4-yl)pyridin-4-amine (1.5 g, 75%) as a yellow solid with LCMS: 90.47% (258.07, [M+H]⁺.

Step 2: 3-nitro-2-(piperazin-1-yl)-N-(tetrahydro-2H-pyran-4-yl)pyridin-4-amine. To a stirred solution of the product of Step 1 (1.5 g, 5.822 mmol), and N-methylpiperizine (0.698 g, 6.987 mmol) in DMSO, was added DIPEA (1.52 mL, 8.733 mmol) and the resulting reaction mixture was heated to 90° C. for 3 h. The reaction was diluted with ice cold water and extracted with ethyl acetate dried over anhydrous Na₂SO₄, filtered and concentrated under reduced pressure. The obtained crude was triturated with n-pentane and the precipitated solid was filtered and dried under vacuo to afford 3-nitro-2-(piperazin-1-yl)-N-(tetrahydro-2H-pyran-4-yl)pyridin-4-amine (1.65 g, 97%) as a yellow solid LCMS: 96.8% (322.24, 317.9[M+H]⁺).

Step 3: 2-(4-methylpiperazin-1-yl)-N4-(tetrahydro-2H-pyran-4-yl)pyridine-3,4-diamine. To a stirred solution of the product of Step 2 (3.7 g, 11.690 mmol) in a mixture of methanol and water (9:1), was added Iron powder (1.97 g ml, 35.480 mmol) and NH₄Cl (3.79 g, 70.956 mmol). The reaction mixture was stirred at 70° C. for 3 h then diluted with methanol, filtered through a pad of celite and the filtrate concentrated under reduced pressure to afford crude 2-(4-methylpiperazin-1-yl)-N4-(tetrahydro-2H-pyran-4-yl)pyridine-3,4-diamine was used for next step without purification.

Step 4: 2-(2-chlorophenyl)-4-(4-methylpiperazin-1-yl)-1-(tetrahydro-2H-pyran-4-yl)-1H-imidazo[4,5-c]pyridine (Compound 1). To a stirred solution of the product of Step 3 (0.25 g, 0.857 mmol) and 2-chlorobenzaldehyde (0.17 g, 1.286 mmol) in ethanol, was added Na₂S₂O₅ (0.4 g, 2.143 mmol) and the resulting reaction mixture was heated to 80° C. for 16 h. The reaction mixture was diluted with methanol, filtered through a pad of celite and the filtrate was concentrated under reduced pressure to give a crude product which was purified by preparative HPLC to afford Compound 1 (0.24 g, 79%) as an off-white solid.

Characterization Data. Melting range: 231-235° C. LCMS: 99.31% (412.34 [M+H]⁺). ¹H NMR (401 MHz, DMSO): δ 7.86 (d, J=5.7 Hz, 1H), 7.67 (m, 3H), 7.54 (t, J=7.4 Hz, 1H), 7.10 (d, J=5.8 Hz, 1H), 4.00 (m, 7H), 3.30 (m, 2H), 2.41 (t, J=4.5 Hz, 4H), 2.19 (s, 5H), 1.80 (s, 2H).

### 2-(2-chlorophenyl)-6-methyl-4-(4-methylpiperazin-1-yl)-1-(tetrahydro-2H-pyran-4-yl)-1H-imidazo[4,5-c]pyridine (Compound 2)

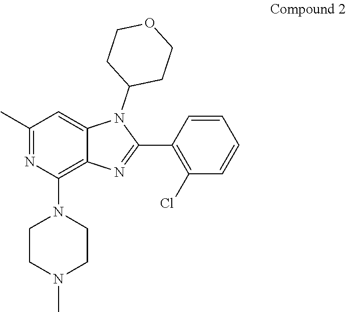 [SMILES: Cc1cc2c(nc(-c3ccccc3Cl)n2C2CCOCC2)c(N2CCN(C)CC2)n1]

2-(2-chlorophenyl)-6-methyl-4-(4-methylpiperazin-1-yl)-1-(tetrahydro-2H-pyran-4-yl)-1H-imidazo[4,5-c]pyridine (Compound 2) was prepared by a similar sequence of reactions to those used to prepare Compound 1, to give an off white solid with melting range: 248-252° C., LCMS: 99.59% (426.23 [M−H]+) and ¹H NMR (401 MHz, DMSO): δ 7.69 (d, J=8.0 Hz, 1H), 7.65-7.60 (m, 2H), 7.53 (t, J=7.3 Hz, 1H), 6.94 (s, 1H), 4.10-3.91 (m, 7H), 3.27 (m, 2H), 2.45-2.18 (m, 12H), 1.85-1.65 (br s, 2H).

### Example 2: Additional Compound Syntheses

### 5-(1-((tert-butyldimethylsilyl)oxy)-2-methylpropan-2-yl)isoxazol-3-amine (Intermediate 1)

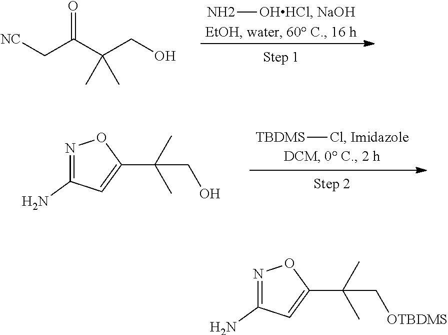

Step 1: 2-(3-aminoisoxazol-5-yl)-2-methylpropan-1-ol. To a stirred solution of 5-hydroxy-4,4-dimethyl-3-oxopentanenitrile (3 g, 21.26 mmol) in a mixture of ethanol and water (1:1), were added NaOH (1 g, 25.08 mmol) and NH₂OH·HCl (1.74 g, 25.08 mmol) and the resulting reaction mixture was stirred at 60° C. for 16 h. Concentrated HCl was then added to the reaction mass and heated at 70° C. for 1 h. Upon completion, the reaction mixture was evaporated to dryness and dissolved in chloroform. The chloroform layer was then basified to pH=12, using 7M NaOH and extracted with 10% MeOH/DCM (10×50 mL) to give crude 2-(3-aminoisoxazol-5-yl)-2-methylpropan-1-ol which was used in the next step without purification.

Step 2: 5-(1-((tert-butyldimethylsilyl)oxy)-2-methylpropan-2-yl)isoxazol-3-amine. A solution of the product of Step 1 (1 g, 6.40 mmol) in DCM, tert-butyldimethylsilyl chloride (1.74 g, 11.5 mmol) and imidazole (871 mg, 12.8 mmol) was stirred at 0° C. After 2 h the reaction was diluted with DCM and washed with 2N HCl. The organic layer was dried over anhydrous sodium sulfate and concentrated under reduced pressure. The crude product was purified by flash chromatography to give 5-(1-((tert-butyldimethylsilyl)oxy)-2-methylpropan-2-yl)isoxazol-3-amine as a yellow oil (1.6 g, (92%). LCMS: 93.13% (271.06 [M+H]⁺). ¹H NMR (401 MHz, DMSO): δ 5.53 (s, 1H), 5.39 (br s, 2H), 3.53 (s, 2H), 1.16 (s, 6H), 0.83 (s, 9H), −0.001 (s, 6H).

### 3-(1-((tert-butyldimethylsilyl)oxy)-2-methylpropan-2-yl)isoxazol-5-amine (Intermediate 2)

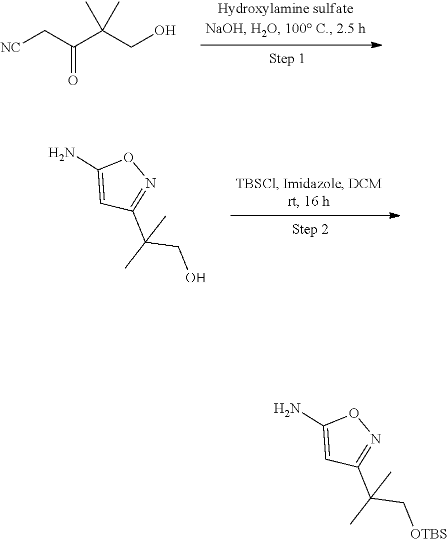

Step 1: 2-(5-aminoisoxazol-3-yl)-2-methylpropan-1-ol. A stirred solution of 5-hydroxy-4,4-dimethyl-3-oxopentanenitrile (1 g, 141.17 mmol), hydroxylamine sulfate (1.16 g, 7.083 mmol) and NaOH (1.13 g, 28.3 mmol) in water (10 mL), was stirred it for 2.5 h at 100° C. The After reaction mixture was then cooled and the product extracted with 10% MeOH/DCM. The organic layer was dried over anhydrous sodium sulfate and concentrated under reduced pressure to give crude 2-(5-aminoisoxazol-3-yl)-2-methylpropan-1-ol which was used for the next step without further purification.

Step 2: 3-(1-((tert-butyldimethylsilyl)oxy)-2-methylpropan-2-yl)isoxazol-5-amine. A solution of the product of Step 1 (470 mg, 156.09 mmol) in DCM (10 mL), imidazole (410 mg, 5.43 mmol) and TBSCl (818 mg, 5.43 mmol) were stirred for 16 h at rt. After completion of the reaction, mixture was quenched with water, worked up with DCM. The organic layer was dried over anhydrous sodium sulfate and concentrated under reduced pressure to give 3-(1-((tert-butyldimethylsilyl)oxy)-2-methylpropan-2-yl)isoxazol-5-amine (0.7 g, 86%). LCMS: 84.20% (271.48 [M+H]⁺).

### N-(3-(tert-butyl)isoxazol-5-yl)-2-(5-(trifluoromethyl)pyridin-2-yl)pyrazolidine-1-carboxamide (Compound 3) 136

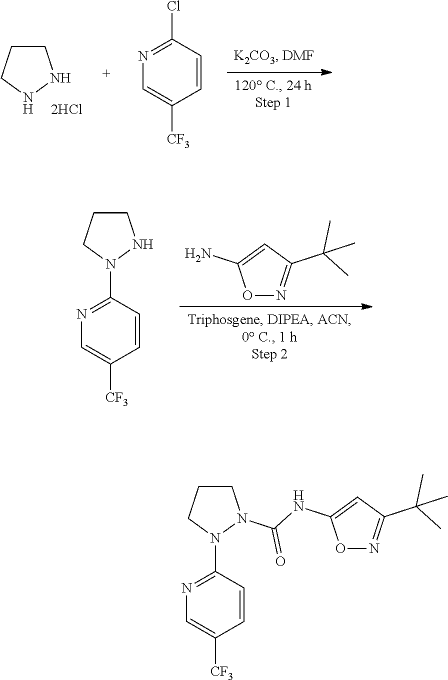

Step 1: 2-(pyrazolidin-1-yl)-5-(trifluoromethyl)pyridine. A mixture of pyrazolidine dihydrochloride (1.0 g, 6.90 mmol), 2-chloro-5-(trifluoromethyl)pyridine (1.4 g, 7.59 mmol) and K₂CO₃ (4.8 g, 34.48 mmol) in DMF (10 mL) was stirred at 120° C. for 24 h. The reaction was then quenched with chilled water (50 mL) and the resulting solid was filtered and dried to afford 2-(pyrazolidin-1-yl)-5-(trifluoromethyl)pyridine (0.5 g) as an off-white solid. LCMS: 84% (218.19, [M+H]⁺

Step 2: N-(3-(tert-butyl)isoxazol-5-yl)-2-(5-(trifluoromethyl)pyridin-2-yl)pyrazolidine-1-carboxamide. A solution of triphosgene (0.170 g, 0.57 mmol), 3-(tert-butyl)isoxazol-5-amine (0.16 g, 1.15 mmol) and DIPEA (0.6 mL, 3.45 mmol) in acetonitrile (15 mL) at −10° C. was stirred for 5 min, then a solution of the product of Step 1 (0.25 g, 1.15 mmol) and DIPEA (0.6 mL, 3.45 mmol) in acetonitrile (10 mL) was added. The reaction mixture was stirred for 1 h at 0° C., quenched with ice water and the product was extracted into EtOAc. The organic layer was dried over anhydrous Na₂SO₄, filtered and concentrated under reduced pressure to give a crude product which was purified by chromatography to give N-(3-(tert-butyl)isoxazol-5-yl)-2-(5-(trifluoromethyl)pyridin-2-yl)pyrazolidine-1-carboxamide (0.045 g) as a white solid. Melting range: 184-188° C.; LCMS: 99.86% (384.19 [M+H]⁺). ¹H NMR (400 MHz, DMSO): δ 10.78 (s, 1H), 8.63 (s, 1H), 7.98 (dd, J=2.0 Hz and 8.8 Hz, 1H), 7.10 (d, J=8.8 Hz, 1H), 6.14 (s, 1H), 4.46 (s, 1H), 4.09 (s, 1H), 3.25 (t, J=9.1 Hz, 1H), 3.11 (d, J=7.4 Hz, 1H), 2.07 (d, J=3.2 Hz, 1H), 1.93 (d, J=5.8 Hz, 1H), 1.24 (s, 9H).

### N-(5-(tert-butyl)isoxazol-3-yl)-2-(5-(trifluoromethyl)pyridin-2-yl)pyrazolidine-1-carboxamide (Compound 4) 134

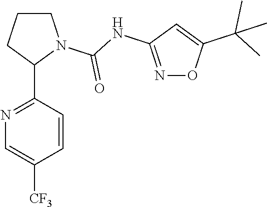 [SMILES: CC(C)(C)c1cc(NC(=O)N2CCCC2c2ccc(C(F)(F)F)cn2)no1]

Prepared in a similar manner as Compound 3. Melting range: 148-152° C., LCMS: 97.02% (382.14 [M−H]⁻). ¹H NMR (400 MHz, DMSO): δ 10.10 (s, 1H), 8.59 (s, 1H), 7.94 (d, J=8.4 Hz, 1H), 7.06 (d, J=8.8 Hz, 1H), 6.52 (s, 1H), 4.39 (br s, 1H), 4.12 (br s, 1H), 3.24-3.04 (m, 2H), 2.04-1.93 (m, 2H), 1.27 (s, 9H).

### N-(3-(tert-butyl)isoxazol-5-yl)-2-(5-(trifluoromethyl)pyrazin-2-yl)pyrazolidine-1-carboxamide (Compound 5) 145

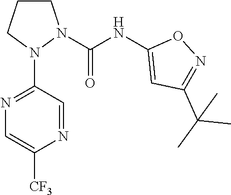 [SMILES: CC(C)(C)c1cc(NC(=O)N2CCCN2c2cnc(C(F)(F)F)cn2)on1]

Prepared in a similar manner as Compound 3. Melting range: 172-176° C., LCMS: 98.86% (383.31 [M−H]⁻). ¹H NMR (400 MHz, DMSO): δ 10.90 (s, 1H), 8.74 (s, 1H), 8.46 (s, 1H), 6.16 (s, 1H), 4.30-4.16 (m, 2H), 3.36-3.31 (m, 1H), 3.16 (br s, 1H), 2.07-2.03 (m, 2H), 1.24 (s, 9H).

### N-(3-cyclopropylisoxazol-5-yl)-2-(5-(trifluoromethyl)pyridin-2-yl)pyrazolidine-1-carboxamide (Compound 6) 160

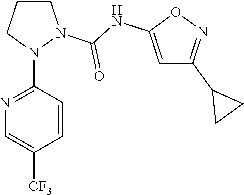 [SMILES: O=C(Nc1cc(C2CC2)no1)N1CCCN1c1ccc(C(F)(F)F)cn1]

Prepared in a similar manner as Compound 3. LCMS: 98.77% (368.55 [M+H]⁺). ¹H NMR (400 MHz, DMSO): δ 10.76 (s, 1H), 8.62 (s, 1H), 7.97 (d, J=8.00 Hz, 1H), 7.08 (d, J=8.80 Hz, 1H), 5.87 (s, 1H), 4.45 (s, 1H), 4.07 (s, 1H), 3.27-3.24 (m, 1H), 3.11-3.09 (m, 1H), 2.00-1.80 (m, 3H), 0.97-0.95 (m, 2H), 0.75-0.70 (m, 2H).

### N-(3-(tert-butyl)-1-methyl-1H-pyrazol-5-yl)-2-(5-(trifluoromethyl)pyridin-2-yl)pyrazolidine-1-carboxamide (Compound 7) 162

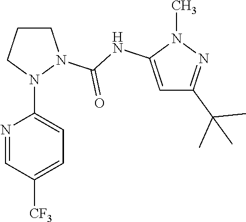 [SMILES: Cn1nc(C(C)(C)C)cc1NC(=O)N1CCCN1c1ccc(C(F)(F)F)cn1]

Prepared in a similar manner as Compound 3. LCMS: 99.14% (395.49 [M−H]−). ¹H NMR (400 MHz, DMSO): δ 9.09 (s, 1H), 8.62 (s, 1H), 8.03-8.00 (m, 1H), 7.19 (d, J=8.8 Hz, 1H), 5.89 (s, 1H), 4.46 (br s, 1H), 4.07 (br s, 1H), 3.50 (s, 3H), 3.31 (br s, 1H), 3.01 (br s, 1H), 2.07-1.95 (m, 2H), 1.19 (s, 9H).

### N-(3-(tert-butyl)isoxazol-5-yl)-2-(5-(trifluoromethyl)pyridin-2-yl)tetrahydropyridazine-1(2H)-carboxamide (Compound 8) 143

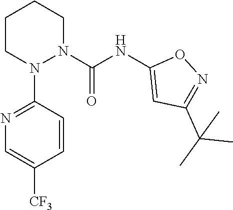 [SMILES: CC(C)(C)c1cc(NC(=O)N2CCCCN2c2ccc(C(F)(F)F)cn2)on1]

Prepared in a similar manner as Compound 3. LCMS: 98.65% (396.33 [M−H]). ¹H NMR (400 MHz, DMSO): δ 10.76 (s, 1H), 8.60 (s, 1H), 7.94 (d, J=7.6 Hz, 1H), 6.95 (d, J=8.8 Hz, 1H), 6.09 (s, 1H), 4.70 (d, J=13.6 Hz, 1H), 4.25 (d, J=12.8 Hz, 1H), 3.05 (q, J=8.4 Hz, 1H), 2.90-2.80 (m, 1H), 1.80-1.60 (m, 4H), 1.23 (s, 9H).

### N-(5-(tert-butyl)isoxazol-3-yl)-2-(5-(trifluoromethyl)pyridin-2-yl)tetrahydropyridazine-1(2H)-carboxamide (Compound 9) 144

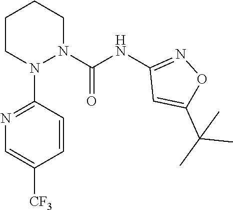 [SMILES: CC(C)(C)c1cc(NC(=O)N2CCCCN2c2ccc(C(F)(F)F)cn2)no1]

Prepared in a similar manner as Compound 3. LCMS: 97.74% (396.33 [M−H]). ¹H NMR (400 MHz, DMSO) δ 10.13 (s, 1H), 8.62 (s, 1H), 7.97 (q, J=2.0 Hz, 1H), 6.95 (d, J=8.4 Hz, 1H), 6.56 (s, 1H), 4.71 (d, J=13.6 Hz, 1H), 4.24 (d, J=12.7 Hz, 1H), 3.04 (m, 1H), 2.80 (m, 1H), 1.61 (m, 4H), 1.28 (s, 9H).

### N-(3-(tert-butyl)isoxazol-5-yl)-2-(5-(trifluoromethyl)pyrazin-2-yl)tetrahydropyridazine-1(2H)-carboxamide (Compound 10) 148

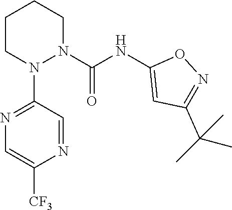 [SMILES: CC(C)(C)c1cc(NC(=O)N2CCCCN2c2cnc(C(F)(F)F)cn2)on1]

Prepared in a similar manner as Compound 3. Melting range: 172-176° C.; LCMS: 97.33% (399.39 [M−H]⁻). ¹H NMR (400 MHz, DMSO): δ 10.85 (s, 1H), 8.62-8.31 (m, 1H), 6.04 (br s, 1H), 4.58 (d, J=12.8 Hz, 1H), 4.31 (s, 1H), 3.05-2.92 (m, 2H), 1.66-1.59 (m, 4H), 1.21 (s, 9H).

### N-(3-(tert-butyl)isoxazol-5-yl)-2-(4-(trifluoromethyl)phenyl)pyrazolidine-1-carboxamide (Compound 11) 139

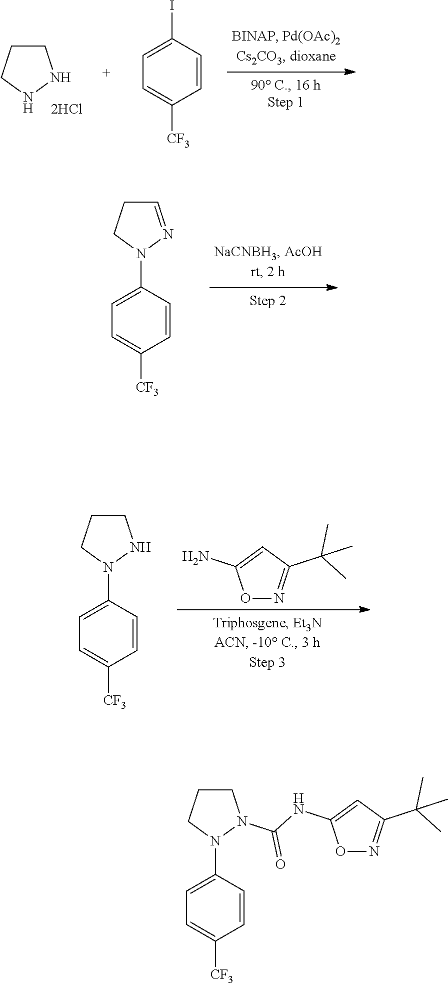

Step 1: 1-(4-(trifluoromethyl)phenyl)-4,5-dihydro-1H-pyrazole. A stirred mixture of pyrazolidine dihydrochloride (1 g, 6.90 mmol), 1-iodo-4-(trifluoromethyl)benzene (2.81 g, 10.34 mmol) and Cs₂CO₃ (6.75 g, 20.69 mmol) in dioxane (30 mL) in a sealed tube was purged with nitrogen gas for 15 minutes. Pd(OAc)₂ (155 mg, 0.689 mmol) and racemic BINAP (0.850 g, 1.38 mmol) was added and, after repurging with nitrogen, the tube was sealed and heated at 90° C. for 16 h. The reaction mixture was then filtered through celite and concentrated under reduced pressure. The residue was treated with water (15 mL) and the product extracted into EtOAc. After drying over anhydrous sodium sulfate and concentrating under reduced pressure, the crude product was purified by chromatography to give 1-(4-(trifluoromethyl)phenyl)-4,5-dihydro-1H-pyrazole (0.8 g) as a white solid. LCMS: 91.1% (215.30, [M+H]⁺. ¹H NMR (400 MHz, CDCl₃): δ 7.50 (d, J=8.4 Hz, 2H), 7.04 (d, J=8.8 Hz, 2H), 6.90 (s, 1H), 3.71 (t, J=10.4 Hz, 2H), 2.99 (t, J=10.0 Hz, 2H).

Step 2: 1-(4-(trifluoromethyl)phenyl)pyrazolidine. A mixture of the product of Step 1 (0.800 g, 3.73 mmol) in acetic acid (2 mL and NaCNBH₃ (0.470 g, 7.47 mmol) was stirred for 2 h at RT. The reaction mixture was then concentrated under reduced pressure to afford crude 1-(4-(trifluoromethyl)phenyl)pyrazolidine which was used in the next step without further purification.

Step 3: N-(3-(tert-butyl)isoxazol-5-yl)-2-(4-(trifluoromethyl)phenyl)pyrazolidine-1-carboxamide. To a stirred solution of triphosgene (0.404 g, 1.3 mmol) in acetonitrile (5 mL) under nitrogen atmosphere at −10° C., was added a mixture of 3-(tert-butyl)isoxazol-5-amine (0.382 g, 2.7 mmol) and trimethylamine (1.9 mL, 14 mmol) in acetonitrile (2 ml) drop wise. After 15 minutes the product of Step 2 (0.450 g, 1.3 mmol) and 5 eq triethylamine in ACN (3 ml) was added drop wise to the reaction mixture. After stirring for 3 h at −10° C. for 3 h, the reaction mixture was quenched with sat NH₄Cl solution at 0° C. and the product extracted into ethyl acetate (60 mL). The organic layer was dried over anhydrous sodium sulfate and concentrated under reduced pressure to give a crude product which was purified by Prep-HPLC to give N-(3-(tert-butyl)isoxazol-5-yl)-2-(4-(trifluoromethyl)phenyl)pyrazolidine-1-carboxamide (20 mg, 12%) as a white solid. Melting range: 211-215° C. LCMS: 99.52% (383.14 [M+H]⁺). ¹H NMR (400 MHz, DMSO): δ 10.57 (s, 1H), 7.61 (d, J=8.4 Hz, 2H), 7.17 (d, J=8.4 Hz, 2H), 6.12 (s, 1H), 4.00 (brs, 1H), 3.78 (brs, 1H), 3.41-3.49 (m, 1H), 3.16 (brs, 1H), 2.07 (brs, 1H), 2.05 (brs, 1H), 1.23 (s, 9H).

### N-(5-(tert-butyl)isoxazol-3-yl)-2-(4-(trifluoromethyl)phenyl)pyrazolidine-1-carboxamide (Compound 12) 140

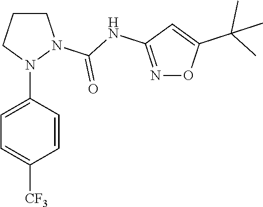 [SMILES: CC(C)(C)c1cc(NC(=O)N2CCCN2c2ccc(C(F)(F)F)cc2)no1]

Prepared in a similar manner as Compound 11. Melting range: 215-219° C., LCMS: 98.93% (381.09 [M−H]+). ¹H NMR (400 MHz, DMSO-d6): δ 9.80 (s, 1H), 7.61 (d, J=8.80 Hz, 2H), 7.16 (d, J=8.40 Hz, 2H), 6.52 (s, 1H), 4.01 (br s, 1H), 3.75 (br s, 1H), 3.40 (brs, 1H), 3.14 (brs, 1H), 2.07 (br s, 1H), 2.04 (br s, 1H), 1.27 (s, 9H).

### N-(3-(4-fluorophenyl)isoxazol-5-yl)-2-(4-(trifluoromethyl)phenyl)pyrazolidine-1-carboxamide (Compound 13) 151

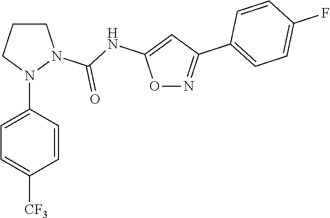 [SMILES: O=C(Nc1cc(-c2ccc(F)cc2)no1)N1CCCN1c1ccc(C(F)(F)F)cc1]

Prepared in a similar manner as Compound 11. Melting range: 184-188° C.; LCMS: 99.10% (419.34 [M−H]⁻). ¹H NMR (400 MHz, DMSO): δ 10.79 (s, 1H), 7.90-7.86 (m, 2H), 7.63 (d, J=8.8 Hz, 2H), 7.32 (t, J=8.8 Hz, 2H), 7.19 (d, J=8.4 Hz, 2H), 6.63 (s, 1H), 4.10-4.00 (br s, 1H), 3.85-3.75 (br s, 1H), 3.45-3.35 (br s, 1H), 3.25-3.15 (br s, 1H), 2.07-1.90 (m, 2H).

### N-(3-(tert-butyl)isoxazol-5-yl)-2-(2-(trifluoromethyl)pyrimidin-5-yl)pyrazolidine-1-carboxamide (Compound 14) 152

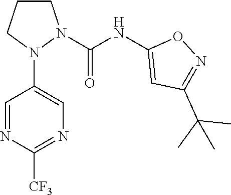 [SMILES: CC(C)(C)c1cc(NC(=O)N2CCCN2c2cnc(C(F)(F)F)nc2)on1]

Prepared in a similar manner as Compound 11. LCMS: 98.89% (385.49 [M+H]⁺). ¹H NMR (400 MHz, DMSO): δ 10.74 (s, 1H), 8.66 (s, 2H), 6.14 (s, 1H), 4.15-3.50 (m, 3H), 3.25-3.10 (m, 1H), 2.05 (t, J=6.4 Hz, 2H), 1.23 (s, 9H).

### N-(3-cyclohexylisoxazol-5-yl)-2-(4-(trifluoromethyl)phenyl)pyrazolidine-1-carboxamide (Compound 15) 153

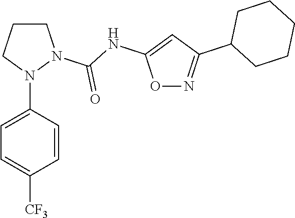 [SMILES: O=C(Nc1cc(C2CCCCC2)no1)N1CCCN1c1ccc(C(F)(F)F)cc1]

Prepared in a similar manner as Compound 11. Melting range: 223-237° C.; LCMS: 93.12% (409.30 [M+H]⁺). ¹H NMR (400 MHz, DMSO): δ 10.56 (s, 1H), 7.61 (d, J=8.8 Hz, 2H), 7.17 (d, J=8.8 Hz, 2H), 6.04 (s, 1H), 3.99 (br s, 1H), 3.79 (br s, 1H), 3.38 (br s, 1H), 3.21 (br s, 1H), 2.65-2.55 (m, 1H), 2.10-1.60 (m, 7H), 1.91 (m, 7H), 1.45-1.20 (m, 5H).

### N-(3-cyclopropylisoxazol-5-yl)-2-(4-(trifluoromethyl)phenyl)pyrazolidine-1-carboxamide (Compound 16) 154

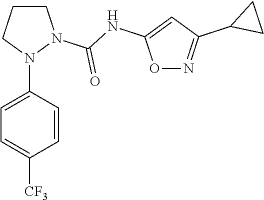 [SMILES: O=C(Nc1cc(C2CC2)no1)N1CCCN1c1ccc(C(F)(F)F)cc1]

Prepared in a similar manner as Compound 11. Melting range: 196-200° C.; LCMS: 93.28% (367.43 [M+H]⁺). ¹H NMR (400 MHz, DMSO): δ 10.55 (s, 1H), 7.61 (d, J=8.8 Hz, 2H), 7.16 (d, J=8.8 Hz, 2H), 5.85 (s, 1H), 3.99 (br s, 1H), 3.78 (br s, 1H), 3.38 (br s, 1H), 3.21 (br s, 1H), 1.93 (m, 3H), 0.98-0.95 (m, 2H), 0.75-0.65 (m, 2H).

### N-(3-(tert-butyl)isoxazol-5-yl)-2-(4-cyanophenyl)pyrazolidine-1-carboxamide (Compound 17) 157

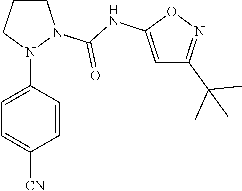 [SMILES: CC(C)(C)c1cc(NC(=O)N2CCCN2c2ccc(C#N)cc2)on1]

Prepared in a similar manner as Compound 11. LCMS: 97.29% (338.37 [M−H]. ¹H NMR (400 MHz, DMSO): δ 10.61 (s, 1H), 7.70 (d, J=8.8 Hz, 2H), 7.11 (d, J=8.8 Hz, 2H), 6.11 (s, 1H), 4.02 (br s, 1H), 3.77 (br s, 1H), 3.36 (br s, 1H), 3.12 (br s, 1H), 2.03-1.90 (m, 2H), 1.23 (s, 9H).

### N-(3-(tert-butyl)isoxazol-5-yl)-2-(4-(trifluoromethyl)phenyl)tetrahydropyridazine-1(2H)-carboxamide (Compound 18) 146

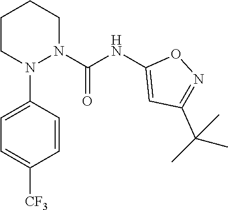 [SMILES: CC(C)(C)c1cc(NC(=O)N2CCCCN2c2ccc(C(F)(F)F)cc2)on1]

Prepared in a similar manner as Compound 11. Melting range: 171-174° C.; LCMS: 99.53% (395.43 [M−H]). ¹H NMR (400 MHz, DMSO): δ 10.65 (s, 1H), 7.62 (d, J=8.4 Hz, 2H), 7.04 (d, J=8.8 Hz, 2H), 6.13 (s, 1H), 4.14 (t, J=15.2 Hz, 2H), 3.21-3.14 (m, 1H), 2.86-2.79 (m, 1H), 1.70-1.50 (m, 4H), 1.24 (s, 9H).

### N-(3-(tert-butyl)isoxazol-5-yl)-2-(3,5-difluorophenyl)pyrazolidine-1-carboxamide (Compound 19) 156

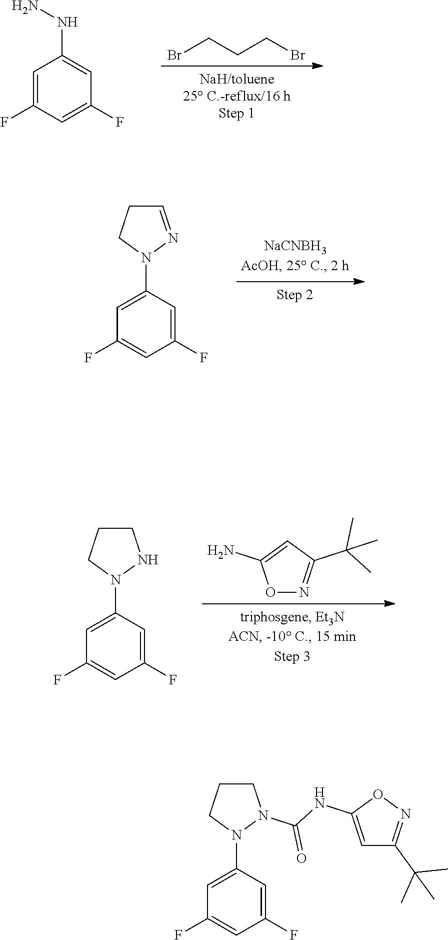

Step 1: 1-(3,5-difluorophenyl)-4,5-dihydro-1H-pyrazole. A mixture of (3,5-difluorophenyl)hydrazine (400 mg, 2.77 mmol) and NaH (57% in oil) (233 mg, 5.54 mmol) in toluene (10 mL) was warmed at 50° C. for 30 mins. Then a portion of 1,3-dibromopropane (196 mg, 0.97 mmol) was added to the reaction mixture at 25° C. and refluxed for 1.5 h. Another portion of 1,3-dibromopropane (196 mg, 0.97 mmol) was added and the reaction mixture was refluxed for a further 16 h. The reaction mixture was quenched with ice and extracted with 10% MeOH/DCM. The organic layer was dried over anhydrous sodium sulfate and concentrated under reduced pressure to obtain a crude product which was purified by Flash chromatography to give 1-(3,5-difluorophenyl)-4,5-dihydro-1H-pyrazole as a yellow oil (260 mg).

Step 2: 1-(3,5-difluorophenyl)pyrazolidine. NaCNBH₃ (179 mg, 2.85 mmol) was added to a stirred solution of the product of Step 1 (260 mg, 1.42 mmol) in acetic acid (3 mL), and the resulting reaction mixture was allowed to stir at 25° C. for 2 h. The reaction mixture was concentrated under reduced pressure to afford crude 1-(3,5-difluorophenyl)pyrazolidine which was used for the next step without further purification.

Step 3: N-(3-(tert-butyl)isoxazol-5-yl)-2-(3,5-difluorophenyl)pyrazolidine-1-carboxamide. To a stirred solution of triphosgene (209 mg, 0.70 mmol) in ACN (5 mL) under nitrogen atmosphere at −10° C., was added a mixture of 3-(tert-butyl)isoxazol-5-amine (197 mg, 1.41 mmol) and triethylamine (1.5 eq) in ACN (5 mL). After 5 mins at −10° C. a mixture of the product of Step 2 (260 mg, 1.41 mmol) and triethylamine (1.5 eq) in ACN (5 mL) was added drop wise in to the reaction mixture and stirring was continued at −10° C. for 15 mins. The reaction, mixture was quenched with sat. NH₄Cl solution at 0° C. and extracted with ethyl acetate. The organic layer was dried over anhydrous sodium sulfate and concentrated under reduced pressure to obtain a crude product which was purified by flash column to give N-(3-(tert-butyl)isoxazol-5-yl)-2-(3,5-difluorophenyl)pyrazolidine-1-carboxamide as a white solid (91 mg). LCMS: 98.75% (351.43 [M+H]⁺); Melting Range: 181-185° C. ¹H NMR (400 MHz, DMSO): δ 10.55 (s, 1H), 6.80-6.68 (m, 3H), 6.11 (s, 1H), 4.00-3.76 (m, 2H), 3.30-3.15 (m, 2H), 2.10-1.90 (m, 2H), 1.23 (s, 9H).

### 2-benzyl-N-(3-(tert-butyl)isoxazol-5-yl)pyrazolidine-1-carboxamide (Compound 20) 155

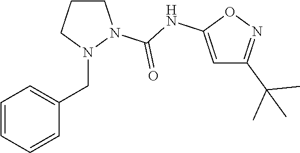 [SMILES: CC(C)(C)c1cc(NC(=O)N2CCCN2Cc2ccccc2)on1]

Prepared by a similar series of reactions as Compound 19. LCMS: 99.46% (329.77 [M+H]+); Melting Range: 143-147° C. ¹H NMR (400 MHz, DMSO): δ 9.48 (s, 1H), 7.48-7.40 (m, 2H), 7.35-7.24 (m, 3H), 5.91 (s, 1H), 3.75 (s, 2H), 3.55 (br s, 2H), 2.93 (br s, 2H), 2.13 (br s, 2H), 1.21 (s, 9H).

### N-(3-(tert-butyl)isoxazol-5-yl)-2-(4-fluorophenyl)pyrazolidine-1-carboxamide (Compound 21) 150

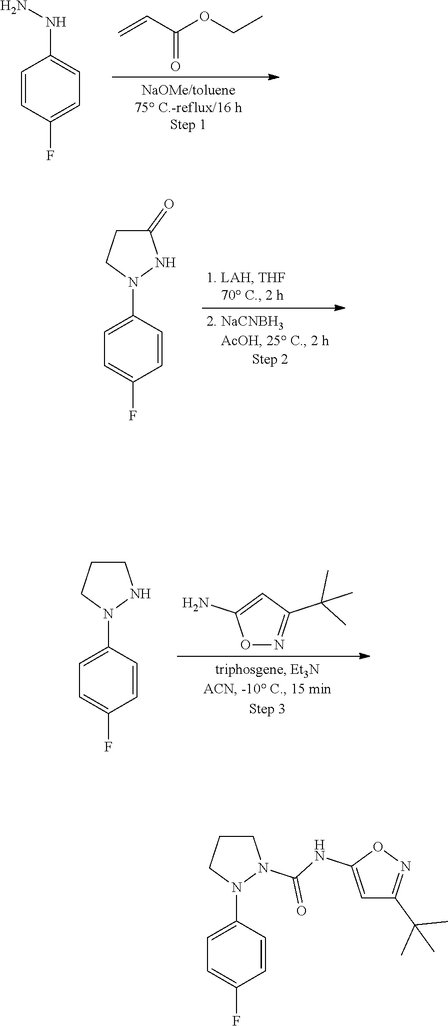

Step 1: 1-(4-fluorophenyl)pyrazolidin-3-one. To a stirred solution of NaOMe (2 g, 15.80 mmol) in toluene (24 mL) and methanol (8 mL), was added a solution of (4-fluorophenyl)hydrazine (400 mg, 2.77 mmol) dissolved in methanol (8 mL), followed by the addition of ethyl acrylate (392 mg, 47.6 mmol) dropwise and the reaction mixture was heated at 75° C. After 16 h the reaction was quenched with ice and extracted with 10% MeOH/DCM. The organic layer was dried over anhydrous sodium sulfate and concentrated under reduced pressure to obtain a crude product which was purified by Flash chromatography afford 1-(4-fluorophenyl)pyrazolidin-3-one (0.7 g, 51%) as a yellow solid LCMS: 46.63% (181.24, [M+H]⁺).

Step 2: 1-(4-fluorophenyl)pyrazolidine. To a stirred solution of the product of Step 1 (700 mg, 3.88 mmol) in THF (25 mL), was added lithium aluminum hydride (1M in THF) (11.66 mL, 11.60 mmol) and the resulting reaction mixture was allowed to stir at 70°. After 2 h the reaction was cooled to 0° C., quenched with aq ammonium chloride and extracted with ethyl acetate. The organic layer was dried over anhydrous sodium sulfate and concentrated under reduced pressure to a yellow gum. This material was dissolved in acetic acid (10 mL), NaCNBH₃ (450 mg, 7.30 mmol) was added and the resulting reaction mixture was stirred at RT. After 3 h the reaction mixture was concentrated under reduced pressure to afford crude 1-(4-fluorophenyl)pyrazolidine which was used for the next step without further purification.

Step 3: N-(3-(tert-butyl)isoxazol-5-yl)-2-(4-fluorophenyl)pyrazolidine-1-carboxamide. To a stirred solution of triphosgene (220 mg, 0.753 mmol) in ACN (10 mL) under nitrogen atmosphere at −10° C., was added a mixture of 3-(tert-butyl)isoxazol-5-amine (210 mg, 1.50 mmol) and triethylamine (1.5 eq) in ACN (2.5 mL) dropwise and the mixture was stirred for 5 min. Then a mixture of the product of Step 2 (250 mg, 1.50 mmol) and triethylamine (2.5 eq) in ACN (2.5 mL) was added dropwise into the reaction mixture. The reaction was quenched with sat. NH₄Cl solution at 0° C. and extracted with ethyl acetate (2×10 mL). The organic layer was dried over anhydrous sodium sulfate and concentrated under reduced pressure to give a crude product which was purified by flash chromatography to afford N-(3-(tert-butyl)isoxazol-5-yl)-2-(4-fluorophenyl)pyrazolidine-1-carboxamide (38 mg, 18%) as white solid

LCMS: 97.13% (331.15 [M−H]⁻), Melting Range: 190-194° C. ¹H NMR (400 MHz, DMSO): δ 10.38 (s, 1H), 7.13-7.02 (m, 4H), 6.09 (s, 1H), 3.90-3.60 (br m, 4H), 2.00-1.95 (br m, 2H), 1.23 (s, 9H).

### N-(5-(1-hydroxy-2-methylpropan-2-yl)isoxazol-3-yl)-2-(4-(trifluoromethyl)phenyl)pyrazolidine-1-carboxamide (Compound 22) 149

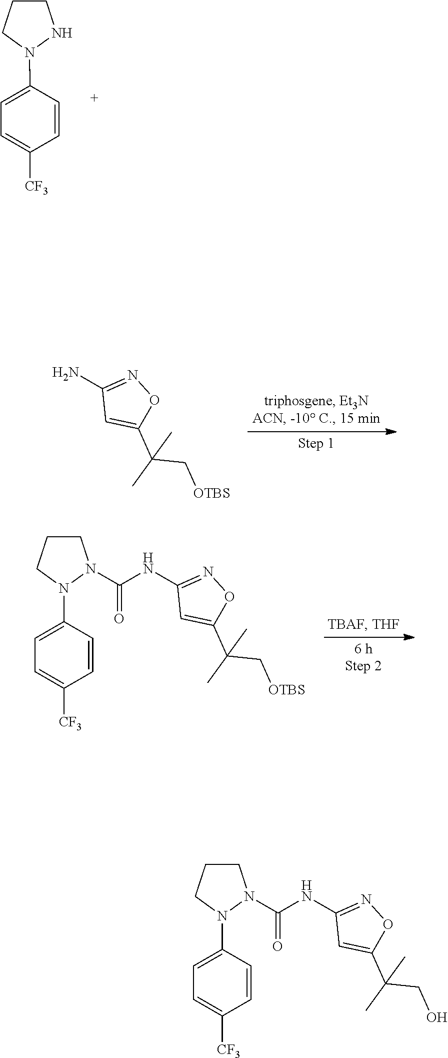

Step 1: N-(5-(1-((tert-butyldimethylsilyl)oxy)-2-methylpropan-2-yl)isoxazol-3-yl)-2-(4-(trifluoromethyl)phenyl)pyrazolidine-1-carboxamide. To a stirred solution of triphosgene (41 mg, 0.13 mmol) in ACN (5 mL) under a nitrogen atmosphere at −10° C., was added a mixture of Intermediate 1 (75 mg, 0.27 mmol) and triethylamine (1.5 eq) in ACN (2.5 mL). The reaction mixture was stirred for 5 mins at −10° C. and a solution of 1-(4-(trifluoromethyl)phenyl)pyrazolidine (60 mg, 0.27 mmol) and triethylamine (1.5 eq) in ACN (2.5 mL) was added drop wise. The reaction mixture was stirred at −10° C. until completion of the reaction. It was then quenched with sat NH₄Cl solution at 0° C. and the product was extracted with ethyl acetate. The organic layer was dried over anhydrous sodium sulfate and concentrated under reduced pressure to give a crude product which was purified by Flash chromatography to give N-(5-(1-((tert-butyldimethylsilyl)oxy)-2-methylpropan-2-yl)isoxazol-3-yl)-2-(4-(trifluoromethyl)phenyl)pyrazolidine-1-carboxamide as an off-white solid (35 mg, 65%). LCMS: 79% (513.47 [M+H]⁻).

Step 2: N-(5-(1-hydroxy-2-methylpropan-2-yl)isoxazol-3-yl)-2-(4-(trifluoromethyl)phenyl)pyrazolidine-1-carboxamide. A solution of the product of Step 1 (35 mg, 0.068 mmol) and TBAF (35 mg, 0.13 mmol) in THF (5 mL) was stirred under nitrogen atmosphere at rt. After 6 hrs the reaction was quenched with sat NH₄Cl solution and extracted with ethyl acetate. The organic layer was dried over anhydrous sodium sulfate and concentrated under reduced pressure to give a crude product which was purified by Flash chromatography to give N-(5-(1-hydroxy-2-methylpropan-2-yl)isoxazol-3-yl)-2-(4-(trifluoromethyl)phenyl)pyrazolidine-1-carboxamide as an off-white solid (16.6 mg, 45%).

LCMS: 98.12% (397.09 [M−H]⁻). Melting Range: 142-146° C. ¹H NMR (400 MHz, DMSO): δ 9.77 (s, 1H), 7.61 (d, J=8.4 Hz, 2H), 7.16 (d, J=8.8 Hz, 2H), 6.55 (s, 1H), 4.93 (t, J=5.5 Hz, 1H), 4.01 (br s, 1H), 3.74 (br s, 1H), 3.42 (d, J=5.2 Hz, 1H), 3.14 (br s, 1H), 2.03-1.93 (m, 2H), 1.20 (s, 6H).

### N-(3-(1-hydroxy-2-methylpropan-2-yl)isoxazol-5-yl)-2-(4-(trifluoromethyl)phenyl)pyrazolidine-1-carboxamide (Compound 23) 158

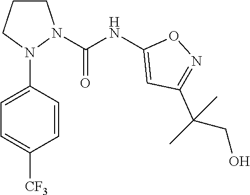 [SMILES: CC(C)(CO)c1cc(NC(=O)N2CCCN2c2ccc(C(F)(F)F)cc2)on1]

Prepared in a similar manner as Compound 22. LCMS: 98.35% (399.29 [M+H]⁺). ¹H NMR (400 MHz, DMSO): δ 10.54 (s, 1H), 7.61 (d, J=8.40 Hz, 2H), 7.16 (d, J=8.40 Hz, 2H), 6.10 (s, 1H), 4.76 (t, J=5.20 Hz, 1H), 4.01 (br s, 1H), 3.78 (br s, 1H), 3.39 (d, J=5.60 Hz, 2H), 3.16 (br s, 2H), 2.05-1.92 (m, 2H), 1.16 (s, 6H).

### N-(3-(1-hydroxy-2-methylpropan-2-yl)isoxazol-5-yl)-2-(5-(trifluoromethyl)pyridin-2-yl)pyrazolidine-1-carboxamide (Compound 24) 159

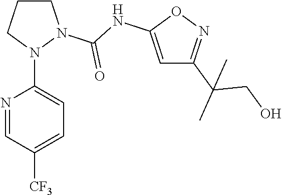 [SMILES: CC(C)(CO)c1cc(NC(=O)N2CCCN2c2ccc(C(F)(F)F)cn2)on1]

Prepared in a similar manner as Compound 22. LCMS: 97.16% (398.43 [M−H]. ¹H NMR (400 MHz, DMSO): δ 10.77 (s, 1H), 8.62 (s, 1H), 7.96 (d, J=7.6 Hz, 1H), 7.06-7.08 (m, 1H), 6.10 (s, 1H), 4.77 (s, 1H), 4.44 (br s, 1H), 4.10 (br s, 1H), 3.39 (d, J=5.6 Hz, 2H), 3.35-3.20 (m, 1H), 3.15-3.05 (m, 1H), 2.10-1.90 (m, 2H), 1.16 (s, 6H).

### N-(3-(2-hydroxypropan-2-yl)isoxazol-5-yl)-2-(4-(trifluoromethyl)phenyl)pyrazolidine-1-carboxamide (Compound 25) 161

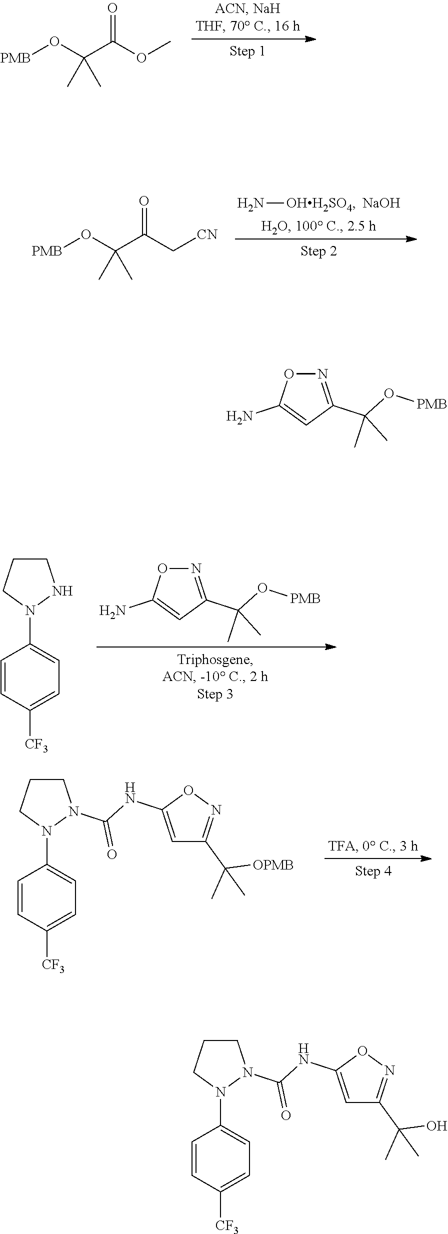

Step 1: 4-((4-methoxybenzyl)oxy)-4-methyl-3-oxopentanenitrile. A stirred solution of NaH (60% in oil) (321 mg, 6.29 mmol) in THF (10 mL) was heated to 70° C. Then a solution of methyl 2-((4-methoxybenzyl)oxy)-2-methylpropanoate (1.0 g, 4.19 mmol) and ACN (0.87 mL, 16.7 mmol) in THF (10 mL) were added drop wise at 70° C. over a period of 1 h. The reaction mixture was then stirred at 75° C. for 15 h. The reaction mixture was diluted with ice cold water and extracted with ethyl acetate (2×50 mL). The combined organic layers were dried over anhydrous sodium sulfate, filtered and concentrated under reduced pressure to obtain a crude product which was purified by flash chromatography to give 4-((4-methoxybenzyl)oxy)-4-methyl-3-oxopentanenitrile as a yellow oil (310 mg, 30%). LCMS: 75% (246.24 [M−H]

Step 2: 3-(2-((4-methoxybenzyl)oxy)propan-2-yl)isoxazol-5-amine. To a stirred solution of the product of Step 1 (310 mg, 1.25 mmol) in water, were added NaOH (200 mg, 5.0 mmol) and NH₂OH·H₂SO₄ (205 mg, 1.25 mmol) and the resulting reaction mixture was stirred at 100° C. for 2.5 h. The reaction mixture was diluted with water and extracted with ethyl acetate. The organic layer was dried over anhydrous sodium sulfate and concentrated under reduced pressure to obtain crude 3-(2-((4-methoxybenzyl)oxy)propan-2-yl)isoxazol-5-amine as a white solid which was used in the next step without further purification.

Step 3: N-(3-(2-((4-methoxybenzyl)oxy)propan-2-yl)isoxazol-5-yl)-2-(4-(trifluoromethyl)phenyl)pyrazolidine-1-carboxamide. To a stirred solution of triphosgene (68.5 mg, 0.23 mmol) in ACN (5 mL) under nitrogen atmosphere at −10° C., was added mixture of the product of Step 2 (121 mg, 0.46 mmol) and triethylamine (1.5 eq) in ACN (5 mL) drop wise. The mixture was stirred for 5 mins and then a mixture of 1-(4-(trifluoromethyl)phenyl)pyrazolidine (100 mg, 0.46 mmol) and triethylamine (1.5 eq) in ACN (5 mL) was added drop wise into the reaction mixture. Then the mixture was stirred at −10° C. for 2 h and then quenched with sat. NH₄Cl solution and extracted with ethyl acetate The organic layer was dried over anhydrous sodium sulfate and concentrated under reduced pressure to give a crude product which was purified by flash chromatography to give N-(3-(2-((4-methoxybenzyl)oxy)propan-2-yl)isoxazol-5-yl)-2-(4-(trifluoromethyl)phenyl)pyrazolidine-1-carboxamide as a brown sticky solid (200 mg, 85%). LCMS: 42.6% (503.5 [M+H]⁺).

Step 4: N-(3-(2-hydroxypropan-2-yl)isoxazol-5-yl)-2-(4-(trifluoromethyl)phenyl)pyrazolidine-1-carboxamide. The product of Step 3 (150 mg, 0.29 mmol) was dissolved in TFA (2.0 mL) and stirred at 0° C. for 3 h. The reaction mixture was concentrated under reduced pressure to afford a crude product which was purified by prep-SFC to afford N-(3-(2-hydroxypropan-2-yl)isoxazol-5-yl)-2-(4-(trifluoromethyl)phenyl)pyrazolidine-1-carboxamide as a white solid (11.7 mg, 10.2%). LCMS: 99.49% (383.10 [M−H]. ¹H NMR (400 MHz, DMSO): δ 10.55 (s, 1H), 7.61 (d, J=8.4 Hz, 2H), 7.16 (d, J=8.4 Hz, 2H), 6.16 (s, 1H), 5.29 (s, 1H), 4.01-3.77 (m, 2H), 3.35-3.16 (m, 2H), 2.07-1.88 (m, 2H), 1.40 (s, 6H).

### N-(3-(2-hydroxypropan-2-yl)isoxazol-5-yl)-2-(4-(trifluoromethyl)phenyl)pyrazolidine-1-carboxamide (Compound 26) 133

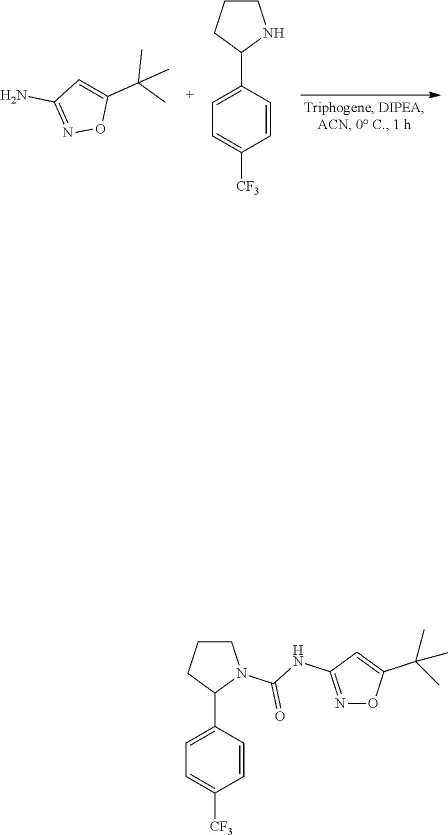

To a stirred solution of triphosgene (0.180 g, 0.892 mmol) in ACN (5 mL) was added a mixture of 5-(tert-butyl)isoxazol-3-amine (0.250 g, 1.785 mmol) and DIPEA (0.6 mL, 5.355 mmol) in ACN at −10° C. After stirring for 5 min at −10° C., a mixture of compound-2 (0.37 g, 1.785 mmol) and DIPEA (0.6 mL, 5.355 mmol) in ACN was added. The reaction mixture was stirred for 1 h and then diluted with ice cold water and EtOAc. The separated organic layer was dried over anhydrous Na₂SO₄, filtered and concentrated under reduced pressure to give a crude product which was purified by flash chromatography followed by Prep.HPLC to give N-(5-(tert-butyl)isoxazol-3-yl)-2-(4-(trifluoromethyl)phenyl)pyrrolidine-1-carboxamide as a white solid (37 mg, 5.4%). LCMS: 99.90% (382.14 [M+H]⁺), Melting range: 175-179° C. ¹H NMR (400 MHz, DMSO): δ 9.46 (s, 1H), 7.67 (d, J=8.0 Hz, 2H), 7.40 (d, J=8.4 Hz, 2H), 6.41 (s, 1H), 5.12 (s, 1H), 3.78 (d, J=4.4 Hz, 1H), 3.57 (d, J=10.0 Hz, 1H), 2.33 (m, 1H), 1.90-1.75 (m, 3H), 1.24 (s, 9H).

### N-(3-(tert-butyl)isoxazol-5-yl)-2-(4-(trifluoromethyl)phenyl)pyrrolidine-1-carboxamide (Compounds 27 and 28) 137 138

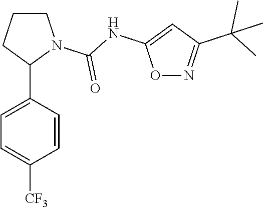 [SMILES: CC(C)(C)c1cc(NC(=O)N2CCCC2c2ccc(C(F)(F)F)cc2)on1]

Prepared in a similar manner to Compound 26. LCMS: 95.28% (380.33 [M−H], Melting range: 169-173° C. ¹H NMR (400 MHz, DMSO): δ 10.09 (s, 1H), 7.67 (d, J=8.4 Hz, 2H), 7.41 (d, J=8.0 Hz, 2H), 5.94 (s, 1H), 5.11 (s, 1H), 3.80-3.75 (m, 1H), 3.61-3.55 (m, 1H), 2.36-2.33 (m, 1H), 1.93-1.71 (m, 3H), 1.23 (s, 9H).

Separation of the racemate into the two pure enantiomers was accomplished with SFC using a Chiralpak IG-3 chiral column. The first eluting enantiomer was Compound 27: LCMS: 98.13% (380.33 [M−H]), Melting range: 189-193° C., Chiral HPLC: 99.77%. The second eluting enantiomer was Compound 28: LCMS: 97.96% (380.33 [M−H]), Melting range: 189-193° C., Chiral HPLC: 99.67%.

### N-(3-(tert-butyl)isoxazol-5-yl)-3-oxo-2-(4-(trifluoromethyl)phenyl)pyrazolidine-1-carboxamide (Compound 29) 129

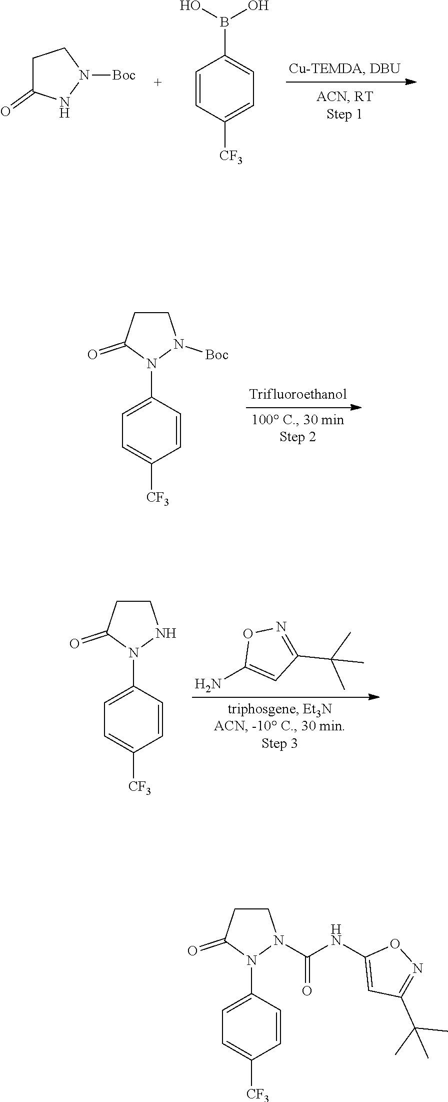

Step 1: tert-butyl 3-oxo-2-(4-(trifluoromethyl)phenyl)pyrazolidine-1-carboxylate. To a stirred solution of tert-butyl 3-oxopyrazolidine-1-carboxylate (1.1 g, 5.91 mmol) in ACN (11 mL) was added DBU (1.35 g, 8.86 mmol). The reaction mixture was stirred at RT for 10 min, after which time Cu-TEMDA (0.274 g, 0.59 mmol) was added and stirred for 10 min Finally (4-(trifluoromethyl)phenyl)boronic acid was added and stirring was continued at RT for 16 h. The reaction mixture was diluted with water and extracted with ethyl acetate. The combined organic layers was washed with brine solution, dried over anhydrous sodium sulfate, filtered and concentrated under reduced pressure to give a crude product which was purified by column chromatography to give tert-butyl 3-oxo-2-(4-(trifluoromethyl)phenyl)pyrazolidine-1-carboxylate as an off-white solid (0.35 g, 18%).

Step 2: 2-(4-(trifluoromethyl)phenyl)pyrazolidin-3-one. A stirred solution of the product of Step 1 (0.35 g, 1.06 mmol) in 2,2,2-trifluoroethanol (2 mL) was stirred at 100° C. under microwave irradiation for 30 min. The reaction mixture was concentrated under reduced pressure to obtain compound-5 as a white solid.

Step 3: N-(3-(tert-butyl)isoxazol-5-yl)-3-oxo-2-(4-(trifluoromethyl)phenyl)pyrazolidine-1-carboxamide. A solution of triphosgene (129 mg, 0.44 mmol) in ACN (5 mL) was cooled to −10° C. To this was added a solution of 3-(tert-butyl)isoxazol-5-amine (98 mg, 0.70 mmol) and TEA (0.2 mL, 1.3 mmol) in ACN (2.5 mL) at −10° C. under nitrogen atmosphere. The reaction mixture was stirred at −10° C. for 10 min, after which time, a solution of the product of Step 2 (200 mg, 0.87 mmol) and TEA (0.2 mL, 1.3 mmol) in ACN (2.5 mL) was added at −10° C. under a nitrogen atmosphere. The reaction mixture was stirred at −10° C. for 30 min and then quenched with water and extracted with EtOAc. The combined organic layers were dried over anhydrous Na₂SO₄, filtered and concentrated under reduced pressure to give a crude product which was purified by Prep HPLC to give N-(3-(tert-butyl)isoxazol-5-yl)-3-oxo-2-(4-(trifluoromethyl)phenyl)pyrazolidine-1-carboxamide as white solid (11.5 mg, 3.2%). LCMS: 92.26% (395.39 [M−H]⁻), Melting Range: 186-190° C. tH NMR (400 MHz, DMSO): δ 11.61 (s, 1H), 7.78 (d, J=8.8 Hz, 2H), 7.72 (d, J=8.8 Hz, 2H), 5.95 (s, 1H), 4.15 (t, J=7.6 Hz, 2H), 2.77 (s, 2H), 1.20 (s, 9H).

### N-(5-(tert-butyl)isoxazol-3-yl)-3-oxo-2-(4-(trifluoromethyl)phenyl)pyrazolidine-1-carboxamide (Compound 30) 131

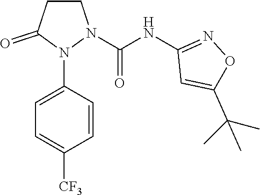 [SMILES: CC(C)(C)c1cc(NC(=O)N2CCC(=O)N2c2ccc(C(F)(F)F)cc2)no1]

Prepared in a similar manner as Compound 29. LCMS: 95.41% (M−H=395.11), Melting Range: 198-202° C. ¹H NMR (400 MHz, DMSO-d6): δ 11.04 (s, 1H), 7.75-7.46 (m, 4H), 6.44 (s, 1H), 4.14 (t, J=7.2 Hz, 2H), 2.81 (t, J=7.2 Hz, 2H), 1.25 (s, 9H).

### N-(5-(tert-butyl)isoxazol-3-yl)-3-oxo-2-(4-(trifluoromethyl)phenyl)pyrazolidine-1-carboxamide (Compound 31) 135

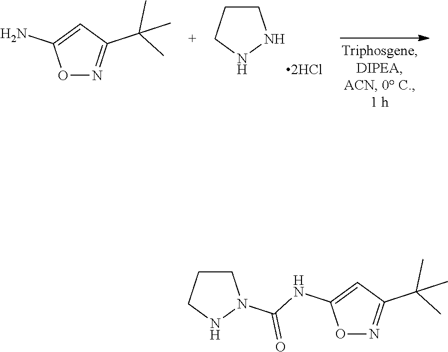

To a stirred solution of triphosgene (0.204 g, 0.68 mmol) in ACN (5 mL) was added a mixture of 3-(tert-butyl)isoxazol-5-amine (0.231 g, 1.785 mmol) and DIPEA (0.6 mL, 5.355 mmol) in ACN at −10° C. After stirring for 5 min, a mixture of pyrazolidine dihydrochloride (0.37 g, 1.785 mmol) and DIPEA (0.6 mL, 5.355 mmol) in ACN, was added. The reaction mixture was stirred for 1 h and then quenched with ice cold water and EtOAc. The separated organic layer was dried over anhydrous Na₂SO₄, filtered and concentrated under reduced pressure to give a crude product which was purified by flash chromatography to give N-(3-(tert-butyl)isoxazol-5-yl)pyrazolidine-1-carboxamide as a white solid (37 mg, 5.4%). LCMS: 98.44% (237.24 [M−H], Melting range: 175-179° C. ¹H NMR (400 MHz, DMSO): δ 9.05 (s, 1H), 6.11 (s, 1H), 3.94 (t, J=8.4 Hz, 1H), 3.60 (br s, 2H), 3.00 (br s, 2H), 2.14 (br s, 2H), 1.31 (s, 9H).

### N-(3-(tert-butyl)isoxazol-5-yl)-2-(oxetan-3-yl)pyrazolidine-1-carboxamide (Compound 32) 163

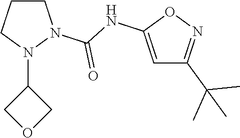 [SMILES: CC(C)(C)c1cc(NC(=O)N2CCCN2C2COC2)on1]

Prepared in a similar manner as Compound 3. LCMS: 94.02% (293.11 [M−H]⁻). ¹H NMR (400 MHz, DMSO): δ 10.37 (s, 1H), 6.05 (s, 1H), 4.52-4.45 (m, 4H), 4.01 (t, J=6.4 Hz, 1H), 3.51 (br s, 2H), 2.81 (t, J=6.8 Hz, 2H), 1.97 (t, J=6.8 Hz, 2H), 1.23 (s, 9H).

### N-(3-(tert-butyl)isoxazol-5-yl)-2-cyclobutylpyrazolidine-1-carboxamide (Compound 33) 164

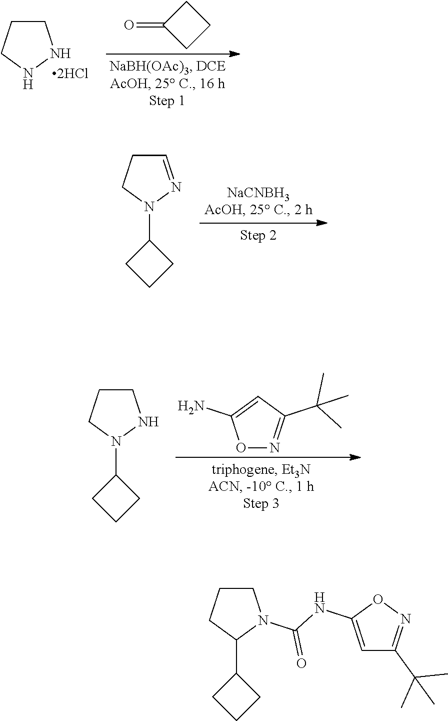

Step 1: To a stirred solution of pyrazolidine dihydrochloride (250 mg, 1.72 mmol) and cyclobutanone (96.3 mg, 1.37 mmol) in 1,2-dichloroethane (10 mL), was added a catalytic amount of AcOH and the reaction was stirred at 25° C. for 1 h. Then NaBH(OAc)₃ (728 mg, 3.44 mmol) was added and the reaction was stirred at 25° C. for 16 h. Reaction progress was monitored by GCMS. Upon completion, the reaction mixture was neutralized with NH₄OH and the complete reaction mixture was concentrated under reduced pressure to afford crude product (1-cyclobutyl-4,5-dihydro-1H-pyrazole). The crude compound was used for the next step without further purification. 296 mg of crude product obtained as a white gum. GCMS: 5.57% (m/z: 124.0).

Step 2: To a stirred solution of the product of Step 1 (250 mg, 2.01 mmol) in acetic acid (3 mL), NaCNBH₃ (252 mg, 4.02 mmol) was added, and the resulting reaction mixture was allowed to stir at 25° C. for 2 h. Reaction progress was monitored by TLC, LCMS. Upon completion, the reaction mixture was concentrated under reduced pressure to afford the crude product (1-cyclobutylpyrazolidine). The crude compound was used for the next step without further purification. 340 mg of desired product was obtained as off-white gum. LCMS: 28.35% (127.09 [M+H]⁺).

Step 3: To a stirred solution of triphosgene (117 mg, 0.39 mmol) in ACN (5 mL) under nitrogen atmosphere at −10° C., was added mixture of 3-(tert-butyl)isoxazol-5-amine (111 mg, 0.79 mmol) and triethylamine (1.5 eq) in ACN (2.5 mL) dropwise. Then the mixture was stirred for 5 mins. After 5 mins a mixture of the product of Step 2 (100 mg, 0.79 mmol) and triethylamine (1.5 eq) in ACN (2.5 mL) was added dropwise in to the reaction mixture. Then the mixture was stirred at −10-25° C. for 1 h. The progress of reaction was monitored by TLC. After completion of reaction, mixture was quenched with sat. NH₄Cl solution at 0° C. and extracted with ethyl acetate (2×10 mL). The organic layer was dried over anhydrous sodium sulphate and concentrated under reduced pressure to obtain the crude. The crude was purified by prep-SFC to afford Compound 35 as a white solid (9.8 mg). LCMS: 98.18% (293.19 [M+H]⁺). ¹H NMR (400 MHz, DMSO): δ 9.97 (s, 1H), 6.03 (s, 1H), 3.60-3.40 (m, 2H), 3.29-3.21 (m, 1H), 2.85-2.75 (br s, 2H), 2.05-1.85 (m, 6H), 1.75-1.65 (m, 1H), 1.60-1.52 (m, 1H), 1.23 (s, 9H).

### N-(3-(3-methyloxetan-3-yl)isoxazol-5-yl)-2-(4-(trifluoromethyl)phenyl)pyrazolidine-1-carboxamide (Compound 34) 165

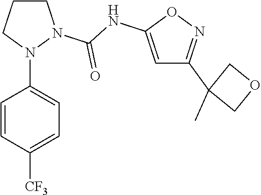 [SMILES: CC1(c2cc(NC(=O)N3CCCN3c3ccc(C(F)(F)F)cc3)on2)COC1]

Prepared in a similar manner as Compound 3. LCMS: 97.29% (397.29 [M+H]⁺). ¹H NMR (400 MHz, DMSO): δ 10.72 (s, 1H), 7.62 (d, J=8.80 Hz, 2H), 7.17 (d, J=8.40 Hz, 2H), 6.24 (s, 1H), 4.74 (d, J=6.00 Hz, 2H), 4.49 (d, J=5.60 Hz, 2H), 4.01 (br s, 1H), 3.80 (br s, 1H), 3.37 (br s, 1H), 3.18 (br s, 1H), 2.12-1.90 (m, 2H), 1.60 (s, 3H).

### N-(3-(1-methoxy-2-methylpropan-2-yl)isoxazol-5-yl)-2-(5-(trifluoromethyl)pyridin-2-yl)pyrazolidine-1-carboxamide (Compound 35), N-(3-(1-hydroxy-2-methylpropan-2-yl)isoxazol-5-yl)-N-methyl-2-(5-(trifluoromethyl)pyridin-2-yl)pyrazolidine-1-carboxamide (Compound 36), and N-(3-(1-methoxy-2-methylpropan-2-yl)isoxazol-5-yl)-N-methyl-2-(5-(trifluoromethyl)pyridin-2-yl)pyrazolidine-1-carboxamide (Compound 37)

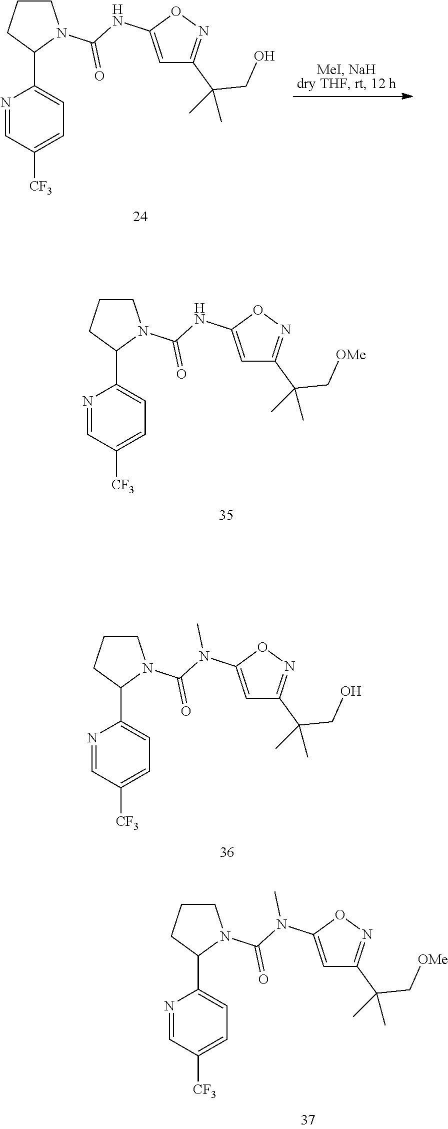 [SMILES: CC(C)(CO)c1cc(NC(=O)N2CCCC2c2ccc(C(F)(F)F)cn2)on1 ; "MeI, NaHdry THF, rt, 12 h" ; COCC(C)(C)c1cc(NC(=O)N2CCCC2c2ccc(C(F)(F)F)cn2)on1 ; CN(C(=O)N1CCCC1c1ccc(C(F)(F)F)cn1)c1cc(C(C)(C)CO)no1 ; COCC(C)(C)c1cc(N(C)C(=O)N2CCCC2c2ccc(C(F)(F)F)cn2)on1]

To a stirred solution of Compound 24 (350 mg, 0.877 mmol) in dry THF (9 mL) under nitrogen atmosphere at −10° C., was added NaH (21 mg, 0.877 mmol) and Mel (149 mg, 1.052 mmol) successively. Then the mixture was warmed to rt and stirred it for 12 h. The progress of reaction was monitored by TLC. After completion of reaction, reaction was quenched with ice-water and extracted with ethyl acetate (3×10 mL). The organic layer was dried over anhydrous sodium sulphate and concentrated under reduced pressure to obtain the crude. The crude was purified by column chromatography followed by SFC-prep to afford the three product compounds. Compound 36 was the major product as a white solid (43.8 mg). Also isolated were Compound 35 (10.49 mg, white solid) and Compound 37 (13.9 mg, light brown, gum).

Compound 35: LCMS: 99.43% (412.08 [M−H]⁻); HPLC: 99.86%, 10.49 mg (white solid). ¹H NMR (400 MHz, DMSO): δ 10.76 (s, 1H), 8.63 (s, 1H), 7.98 (dd, J=2.40 Hz and 8.80 Hz, 1H), 7.10 (d, J=8.80 Hz, 1H), 6.13 (s, 1H), 4.46 (br s, 1H), 4.09 (br s, 1H), 3.34 (s, 2H), 3.30-3.22 (m, 4H), 3.15-3.05 (m, 1H), 2.10-2.00 (m, 1H), 2.00-1.85 (m, 1H), 1.21 (s, 6H).

Compound 36: 43.80 mg as a white solid. LCMS: 414.16 [M+H]⁺. 1H NMR (400 MHz, DMSO: δ 8.48 (s, 1H), 7.91-7.88 (dd, J=2.40 Hz and 8.80 Hz, 1H), 6.91 (d, J=8.80 Hz, 1H), 6.10 (s, 1H), 4.72 (t, J=5.60 Hz, 1H), 4.00-3.40 (br m, 6H), 3.33 (d, J=5.6 Hz, 3H), 2.05-1.95 (m, 2H), 1.09 (s, 6H).

Compound 37: LCMS: 99.06% (428.17 [M+H]⁺), 13.9 mg, light brown gum. ¹H NMR (400 MHz, DMSO): δ 8.48 (s, 1H), 7.91-7.87 (dd, J=2.40 Hz and 8.80 Hz, 1H), 6.91 (d, J=8.80 Hz, 1H), 6.10 (s, 1H), 3.90-3.50 (br m, 2H), 3.28 (s, 3H), 3.26 (s, 4H), 3.21 (s, 3H), 2.06-1.98 (m, 2H), 1.13 (s, 6H).

### N-(5-(tert-butyl)isoxazol-3-yl)-2-oxo-5′-(trifluoromethyl)-2H-[1,2′-bipyridine]-5-carboxamide (Compound 38)

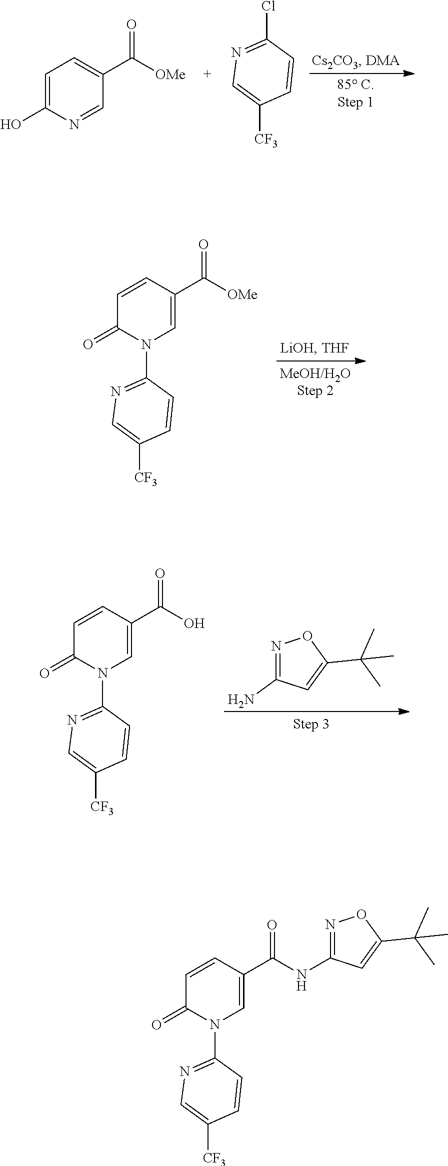

Step 1: A stirred solution of methyl 6-hydroxynicotinate (1.0 g, 6.90 mmol), 2-chloro-5-(trifluoromethyl)pyridine (1.4 g, 7.59 mmol) and Cs₂CO₃ (4.8 g, 34.48 mmol) in DMF (10 mL) was allowed to stir at 120 C.° for 5 h while progress was monitored by LCMS; upon completion, reaction mixture was quenched with chilled water (50 mL) and stirred for 10 min; the resulting solid was collected by filtration and dried to afford the product (methyl 2-oxo-5′-(trifluoromethyl)-2H-[1,2′-bipyridine]-5-carboxylate) (0.8 g) as an off white solid. LCMS: 93% (299.39, [M+H]⁺).

Step 2: To a solution of the product of step 1 (0.8 g, 2.60 mmoles) in THF:MeOH:H₂O (30 mL), was added LiOH·H₂O (0.56 g, 13.42 mmoles) and the reaction was stirred at rt for 2 h. Reaction was monitored by TLC. After completion, the reaction mass was acidified with 1N Hydrochloric acid up to pH=4 and the resulting solid was filtered and dried to obtain the product (2-oxo-5′-(trifluoromethyl)-2H-[1,2′-bipyridine]-5-carboxylic acid). Yield: 0.45 g.

Step 3: solution of the product of step 2 (0.1 g, 0.33 mmol) in SOCl₂ (1 mL) was refluxed for 2 h and evaporated SOCl₂ on rotovapor and dried. In another flask a solution of 5-(tert-butyl)isoxazol-3-amine in THF, cooled to 0° C., was added NaH and stirred for 30 min; then above prepared acid chloride was slowly added and stirring was continued for 16 h at RT. After completion of reaction, solvent was evaporated on rotavapor to obtain crude compound. This crude compound was purified by Prep.HPLC to obtain Compound 38 as white solid. Yield: 0.018 g (13.8%). LCMS: 99.84% (407.37 [M−H]). ¹H NMR (400 MHz, DMSO): δ 11.28 (s, 1H), 9.12 (s, 1H), 8.89 (d, J=2.4 Hz, 1H), 8.51-8.48 (m, 1H), 8.11-8.07 (m, 2H), 6.68 (s, 1H), 6.66 (d, J=10.0 Hz, 1H), 1.31 (s, 9H).

### N-(5-cyclopropylisoxazol-3-yl)-2-(4-(trifluoromethyl)phenyl)pyrazolidine-1-carboxamide (Compound 39)

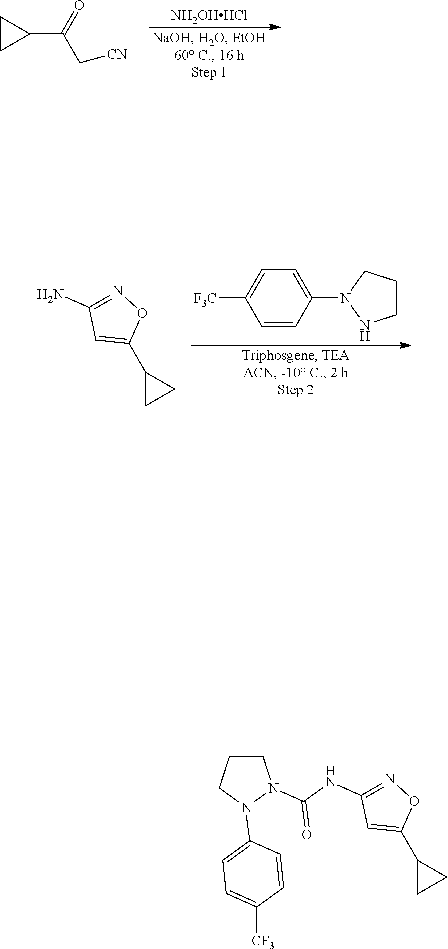

Step 1: To a stirred solution of 3-cyclopropyl-3-oxopropanenitrile (200 mg, 1.83 mmol) in water/EtOH (1:1, 10 mL), hydroxylamine hydrochloride (152 mg, 2.19 mmol) and NaOH (87.6 mg, 2.19 mmol) were added and then stirred for 16 h at 60° C. After 16 h, concentrated HCl was added to the reaction mass and heated at 80° C. for 1 h. Upon completion, reaction mixture was evaporated to dryness and dissolved in chloroform, basified to pH=12, using 7M NaOH and concentrated to obtain the crude. The crude was purified by Flash chromatography (using silica (mesh-100-200), 20% EtOAc in Pet Ether to get the product (5-cyclopropylisoxazol-3-amine) as a brown oil (80 mg). LCMS: 75.92% (124.89 [M+H]⁺). ¹H NMR (400 MHz, CDCl₃): δ 5.47 (s, 1H), 3.85 (br s, 2H), 1.92-1.88 (m, 1H), 1.02-0.96 (m, 4H).

Step 2: To a stirred solution of triphosgene (94.7 mg, 0.32 mmol) in ACN (3 mL) under nitrogen atmosphere at −10° C., a mixture of the product of Step 1 (80.3 mg, 0.64 mmol) and triethylamine (5 eq) in ACN (2 mL) was added dropwise. Then the mixture was stirred for 5 min. After 5 min a mixture of 1-(4-(trifluoromethyl)phenyl)pyrazolidine (140 mg, 0.64 mmol) and triethylamine (5 eq) in ACN (2 mL) was added dropwise into the reaction mixture. Then the mixture was stirred at −10° C. for 2 h. The progress of reaction was monitored by TLC. After completion of reaction, reaction was quenched with water at 0° C. and extracted with ethyl acetate (2×10 mL). The organic layer was dried over anhydrous sodium sulphate and concentrated under reduced pressure to obtain the crude. The crude was purified by prep-SFC to afford pure Compound 39 as an off-white solid (9.02 mg). LCMS: 98.20% (367.35 [M+H]⁺); UPLC: 98.89% ¹H NMR (400 MHz, DMSO-d6): δ 9.75 (s, 1H), 7.61 (d, J=8.40 Hz, 2H), 7.16 (d, J=8.40 Hz, 2H), 6.50 (s, 1H), 4.02 (br s, 1H), 3.74 (br s, 1H), 3.34 (br s, 1H), 3.13 (br s, 1H), 2.12-1.90 (m, 3H), 1.04-1.01 (m, 2H), 0.85-0.84 (m, 2H).

### N-(3-(tert-butyl)-1,2,4-oxadiazol-5-yl)-2-(4-(trifluoromethyl)phenyl)pyrazolidine-1-carboxamide (Compound 40)

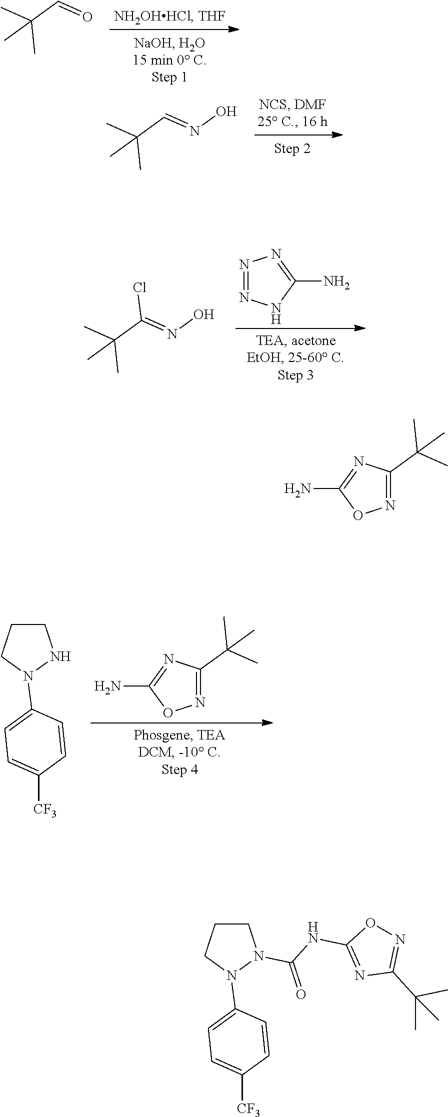

Step 1: To a stirred solution of pivalaldehyde (2 g, 23.33 mmol) in THF/H₂O (1:1) at 0° C., NH₂OH·HCl and NaOH were added, and the reaction mixture was stirred at 0° C. for 15 mins. Reaction progress was monitored by LCMS. After completion of reaction, solvent was evaporated from the reaction mixture under reduced pressure and extracted with ethyl acetate (2×100 mL). The organic layer was dried over anhydrous sodium sulphate and concentrated under reduced pressure to obtain the crude compound (pivalaldehyde oxime). The crude compound was used for the next step without further purification. Colorless oil (1.8 g, crude). ¹H NMR 400 MHz, DMSO-d6: δ 10.33 (s, 1H), 7.24 (s, 1H), 1.03 (s, 9H).

Step 2: To a stirred solution of the product of Step 1 (1.8 g 17.7 mmol) in DMF (15 mL), N-chlorosuccinimide (NCS) (2.49 g, 18.6 mmol) was added, and the resulting reaction mixture was allowed to stir at 25° C. for 16 h. Reaction progress was monitored by TLC. After completion of reaction, the reaction mixture was diluted with water and extracted with ethyl acetate (2×100 mL). The organic layer was washed with ice-water, dried over anhydrous sodium sulfate and concentrated under reduced pressure to obtain the crude product (N-hydroxypivalimidoyl chloride). The crude compound was used for the next step without further purification. Brown oil (1.3 g, crude). ¹H NMR 400 MHz, DMSO-d6: δ 11.53 (s, 1H), 1.19 (s, 9H).

Step 3: To a stirred solution of the product of Step 2 (1.3 g, 9.60 mmol) and 1H-tetrazol-5-amine (817 mg, 9.60 mmol) in acetone (10 mL), TEA (0.5 eq) was added and the resulting reaction mixture was allowed to stir at 25° C. for 30 mins. After 30 mins, TLC showed that the 1H-tetrazol-5-amine was consumed. The reaction mass was evaporated under reduced pressure to remove the volatiles. The crude mass was again dissolved in ethanol and TEA (0.5eq) was added to it and heated at 50° C. for 6.5 h. Reaction progress was monitored by TLC. After completion of reaction, the reaction mixture was diluted with water and extracted with ethyl acetate (2×100 mL). The organic layer was dried over anhydrous sodium sulphate and concentrated under reduced pressure to obtain the crude compound (3-(tert-butyl)-1,2,4-oxadiazol-5-amine). The obtained crude was purified by Flash chromatography (100-200 mesh silica, eluted with 30% EtOAc in Pet ether) to afford the product as a white solid (0.60 g). LCMS: 93.69% (141.89 [M+H]⁺). ¹H NMR δ 400 MHz, DMSO-d6: δ 7.59 (s, 2H), 1.21 (s, 9H).

Step 4: To a stirred solution of Phosgene (20% in toluene) (0.22 mL, 0.46 mmol) in DCM (5 mL) under nitrogen atmosphere at −10° C., was added mixture of the product of Step 3 (131 mg, 0.92 mmol) and triethylamine (1.5 eq) in DCM (2.5 mL) dropwise. Then the mixture was stirred for 5 min. After 5 mins a mixture of 1-(4-(trifluoromethyl)phenyl)pyrazolidine (100 mg, 0.46 mmol) and triethylamine (1.5 eq) in DCM (2.5 mL) was added dropwise into the reaction mixture. Then the mixture was stirred at −10° C. for 1 h. The progress of reaction was monitored by TLC. After completion of reaction, reaction was quenched with sat. NH₄Cl solution at 0° C. and extracted with ethyl acetate (2×10 mL). The organic layer was dried over anhydrous sodium sulphate and concentrated under reduced pressure to obtain the crude. The crude was purified by prep-SFC to afford Compound 40 as a white solid (26.13 mg). LCMS: 98.22% (382.22 [M−H]⁻). UPLC: 98.51% ¹H NMR 400 MHz, DMSO-d6: δ 11.21 (br s, 1H), 7.56 (d, J=8.80 Hz, 2H), 7.08 (d, J=8.40 Hz, 2H), 4.15-4.05 (m, 1H), 3.75-3.60 (m, 1H), 3.45-3.35 (m, 1H), 3.15-3.05 (m, 1H), 2.10-1.90 (m, 2H), 1.23 (s, 9H).

### N-(3-(trifluoromethyl)isoxazol-5-yl)-2-(5-(trifluoromethyl)pyridin-2-yl)pyrazolidine-1-carboxamide (Compound 41)

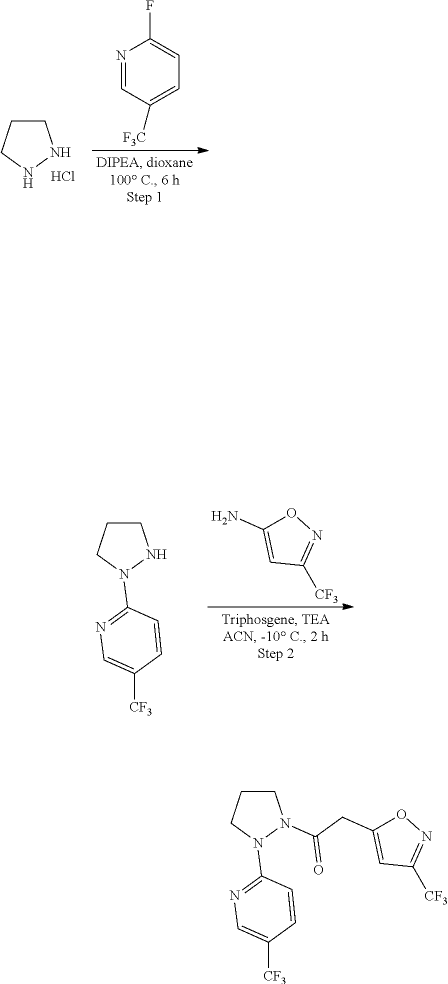 [SMILES: C1CNNC1 ; Fc1ccc(C(F)(F)F)cn1 ; Cl ; FC(F)(F)c1ccc(N2CCCN2)nc1 ; Nc1cc(C(F)(F)F)no1 ; O=C(Cc1cc(C(F)(F)F)no1)N1CCCN1c1ccc(C(F)(F)F)cn1]

Step 1: To a stirred solution of pyrazolidine hydrochloride (3 g, 20.6 mmol) and 2-fluoro-5-(trifluoromethyl)pyridine (3.4 g, 20.6 mmol) in dioxane (15 mL), DIPEA (14.3 mL, 82.4 mmol) was added, and the resulting reaction mixture was allowed to stir at 120° C. for 6 h in sealed tube. After completion of reaction, water was added to the reaction mixture and extracted with ethyl acetate (3×20 mL). The organic layer was dried over anhydrous sodium sulfate and concentrated under reduced pressure to obtain the crude product (2-(pyrazolidin-1-yl)-5-(trifluoromethyl)pyridine). The crude was used in the next step without further purification. White solid (3.5 g). LCMS: 85.31% (218.04, [M+H]⁺. ¹H NMR (400 MHz, CDCl₃): δ 8.34 (s, 1H), 7.73-7.70 (dd, J=2.4 Hz and 8.8 Hz, 1H), 7.08 (d, J=9.2 Hz, 1H), 5.14 (t, J=8.4 Hz, 1H), 3.55 (t, J=5.4 Hz, 2H), 2.89-2.83 (m, 2H), 2.06-1.99 (m, 2H).

Step 2: To a stirred solution of triphosgene (34.4 mg, 0.11 mmol) in ACN (2 mL) under nitrogen atmosphere at −10° C., was added mixture of 3-(trifluoromethyl)isoxazol-5-amine (35.2 mg, 0.23 mmol) and triethylamine (5 eq) in ACN (1 mL) dropwise. Then the mixture was stirred for 5 min. After 5 min, a mixture of the product of Step 1 (50 mg, 0.23 mmol) and triethylamine (5 eq) in ACN (1 mL) was added dropwise into the reaction mixture. Then the mixture was stirred at −10° C. for 2 h. The progress of reaction was monitored by TLC. After completion of reaction, mixture was quenched with water at 0° C. and extracted with ethyl acetate (2×10 mL). The organic layer was dried over anhydrous sodium sulphate and concentrated under reduced pressure to obtain the crude. The crude was purified by preparative SFC to afford Compound 41 as a white solid (17.6 mg). LCMS: 98.64% (394.23 [M+H]⁺); UPLC: 99.13% ¹H NMR (400 MHz, DMSO): δ 11.43 (s, 1H), 8.64 (s, 1H), 7.98 (d, J=8.00 Hz, 1H), 7.13 (d, J=8.8 Hz, 1H), 6.55 (br s, 1H), 4.48 (br s, 1H), 4.09 (br s, 1H), 3.30-3.10 (m, 2H), 2.15-2.08 (m, 1H), 1.90-1.70 (m, 1H).

### Example 2: CB1R/CB2R Activity

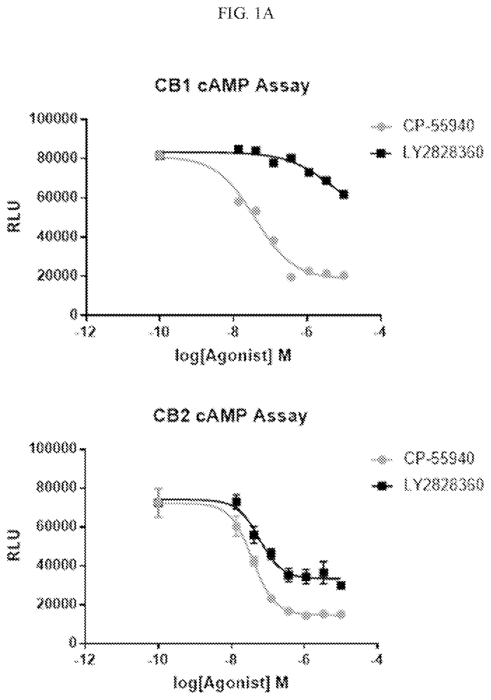

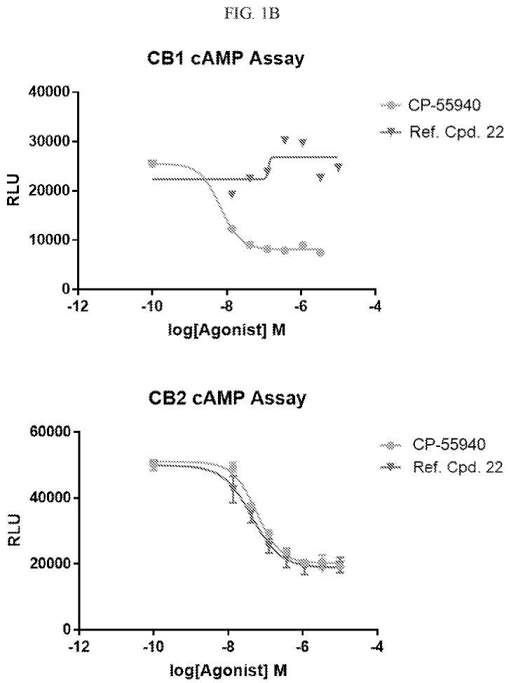

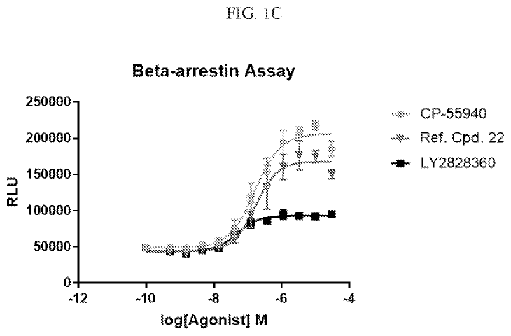

CHO cells expressing human CB1R or CB2R were used to determine EC50 values for the selective CB2R agonists LY2828360 and Reference Compound 22 (Compound 22 from Riether et al. Bioorg. Med. Chem. Lett. (2015) 25(3):581-586) in a cAMP assay, with the full agonist CP-55940 as the control. cAMP assays were performed using the cAMP Hunter™ eXpress GPCR Assay for human CB1 (catalog no. 95-0071E2CP2L) and human CB2 (catalog no. 95-0183E2CP2L) (source: DiscoveRx, Fremont, CA, USA), according to the manufacturer's protocol (https://www.discoverx.com/tools-resources/document-resource-library/documents/user-manual-camp-hunter-express-gpcr-assay). Briefly, cAMP Hunter eXpress Cells were seeded 18-24 h prior to the in a 96-well tissue culture treated assay plate and incubated in 5% CO2 at 37° C. in a humidified incubator. After removal of cell media, agonist compound and forskolin prepared in cell assay buffer was added and incubated at 37° C. The CB1 cAMP assay was stimulated with 20 μM final concentration of forskolin with 40 minutes incubation time. The CB2 cAMP assay was stimulated with 25 μM final concentration of forskolin with 35 minutes incubation time. The assay was stopped by incubation with antibody and working solution for 1 h at room temperature. After over 3 h incubation at room temperature in the dark with enzyme acceptor solution, chemiluminescence was measured on a CLARIOstar plate reader (BMG LABTECH Inc., Cary, NC, USA). The EC50 value is the concentration at which 50% of the forskolin-stimulated cAMP synthesis is inhibited by the compound. Results are shown in FIGS. 1A-1B, and in Tables 1 and 2 below.

TABLE 1

| | CB2 EC50 | CB2 Maximum |
|---|---|---|
| Compounds | (nM) | Activation |
| CP-55940 | 37.55 | 100% |
| LY2828360 | 68.18 | 52.64%% |

TABLE 2

| | CB2 EC50 | CB2 Maximum |
|---|---|---|
| Compounds | (nM) | Activation |
| CP-55940 | 52.92 | 100% |
| Ref. Cpd. 22 | 42.73 | 99.68% |

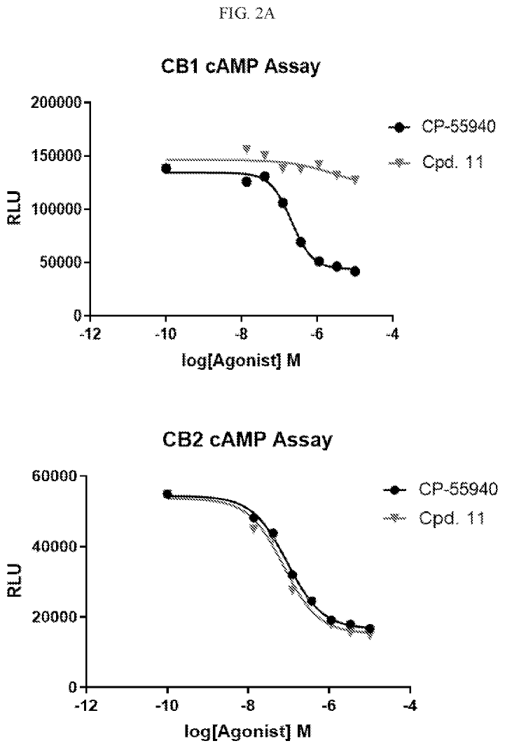

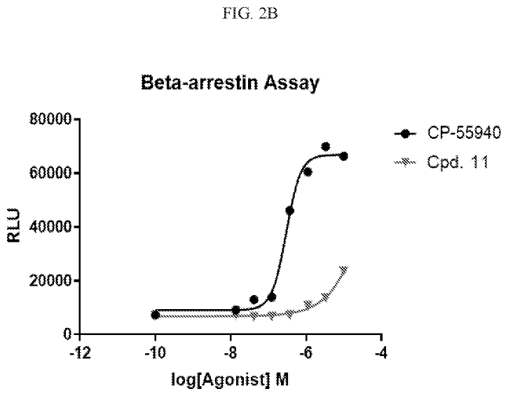

The experiments were repeated using Compound 11 (N-(3-(tert-butyl)isoxazol-5-yl)-2-(4-(trifluoromethyl)phenyl)pyrazolidine-1-carboxamide), with the full agonist CP-55940 as the control. Results are shown in FIG. 2A, and in Table 3 below. The data show that Compound 11 is selective for CB2 over CB1 and is a full agonist comparable to the full agonist control, CP-55940.

TABLE 3

| | CB2 EC50 | CB2 Maximum |
|---|---|---|
| Compounds | (nM) | Activation |
| CP-55940 | 91.59 | 100% |
| Compound 11 | 75.15 | 114.5%% |

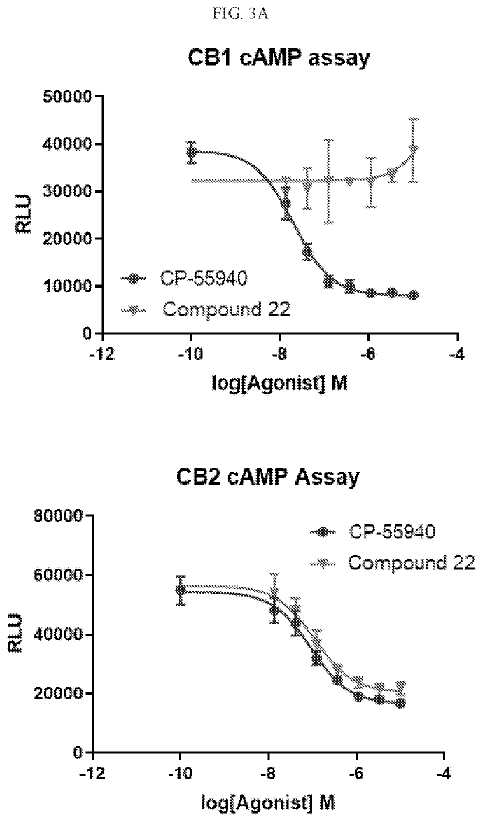

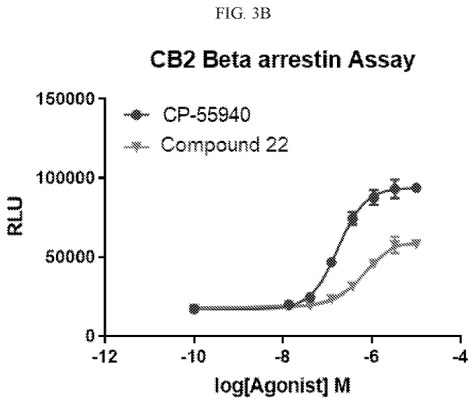

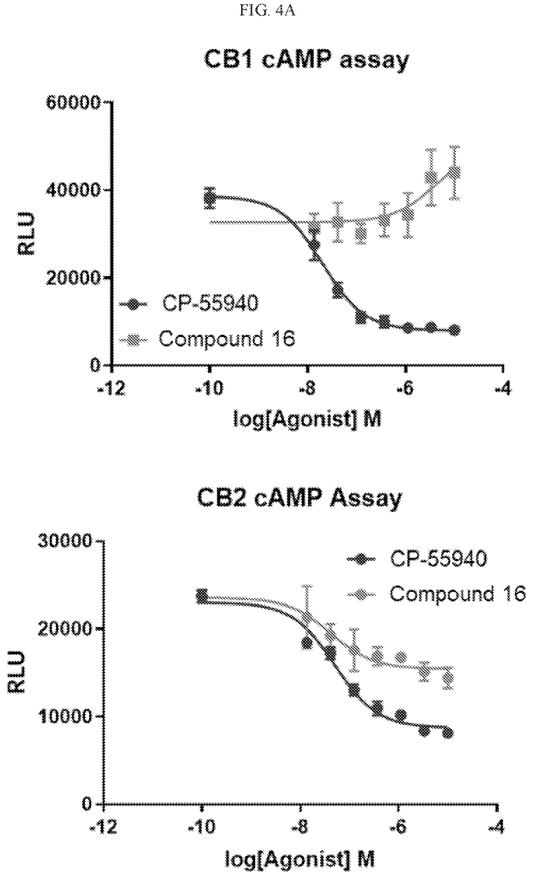

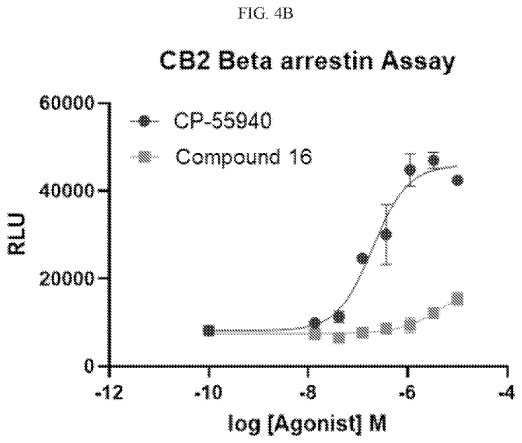

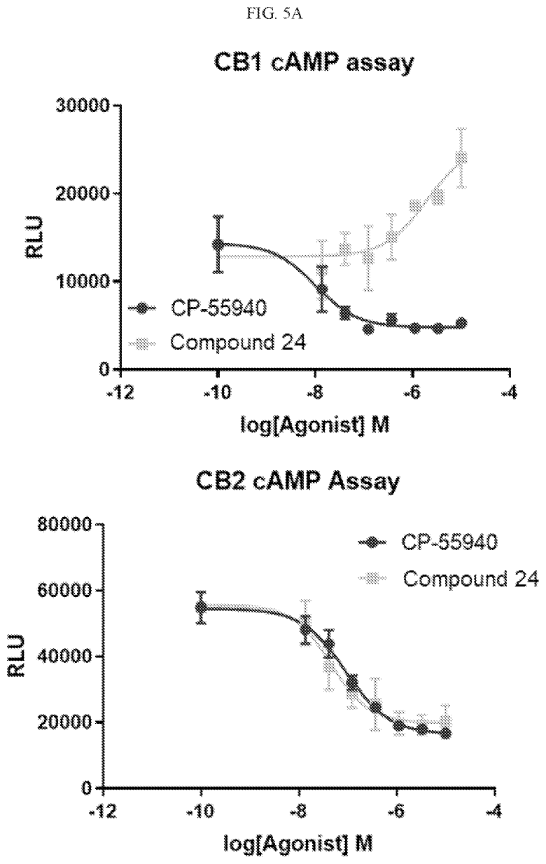

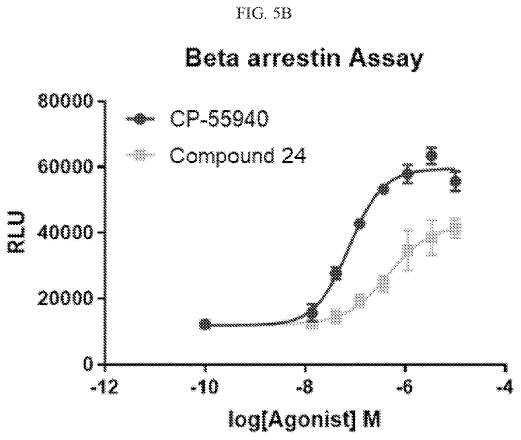

The experiments were also repeated using Compounds 22, 16, and 24, with the full agonist CP-55940 as the control. Results are shown in FIGS. 3A, 4A, and 5A, respectively. The data show that Compounds 22, 16, and 24 are selective for CB2 over CB1. Compounds 22 and 24 are potent agonists of CB2, and Compound 16 is a partial agonist of CB2.

### Example 3: Beta-Arrestin Assay

This assay shows beta-arrestin recruitment at the hCB2 receptor. The assay was performed using the PathHunter β-Arrestin human CB2 cell line (catalog no. 93-0706C2) and using the PathHunter® β-Arrestin Assay for GPCR Cell Lines (source: DiscoveRx, Fremont, CA, USA), according to the manufacturer's protocol (https://www.discoverx.com/tools-resources/document-resource-library/documents/user-manual-pathhunter-% CE % B2-arrestin-assay-for-gpcr-c). Briefly, cells were seeded at a density of 10,000 cells per well in a 96-well plate and incubated overnight (16-18 h) in a humidified atmosphere at 37° C. and 5% C₀₂. Agonist prepared in PBS was added and the assay plate incubated for 3 h in a humidified atmosphere at 37° C. and 5% C₀₂. Working detection solution was added and the plate was incubated for 1 h in the dark at room temperature. Chemiluminescence was measured on an CLARIOstar plate reader (BMG LABTECH Inc., Cary, NC, USA). LY2828360 weakly/partially activates the beta-arrestin pathway while Reference Compound 22 also partially activates beta-arrestin pathway, but more potently than LY2828360. Data are shown in FIGS. 1C and 1n Table 4.

TABLE 4

| | CB2 EC50 | CB2 Maximum |
|---|---|---|
| Compounds | (nM) | Activation |
| CP-55940 | 163.5 | 100% |
| Ref. Cpd. 22 | 166.6 | 81.49% |
| LY2828360 | 48.82 | 51.66% |

The experiments were repeated using Compound 11 (N-(3-(tert-butyl)isoxazol-5-yl)-2-(4-(trifluoromethyl)phenyl)pyrazolidine-1-carboxamide), with the full agonist CP-55940 as the control. Results are shown in FIG. 2B, and in Table 5 below. The data show that Compound 11 weakly/partially activates the beta-arrestin pathway, but is substantially less potent than CP-55940.

TABLE 5

| | | B-arrestin |
|---|---|---|
| | B-arrestin | Maximum |
| Compounds | EC50 (nM) | Activation |
| CP-55940 | 299.6 | 100% |
| Compound 11 | >1,000 | 35.5% |

The experiments were also repeated using Compounds 22, 16, and 24, with the full agonist CP-55940 as the control. Results are shown in FIGS. 3B, 4B, and 5B, respectively. The data show that Compound 16 has almost no beta-arrestin activity, while Compounds 22 and 24 have weak beta-arrestin activity.

### Example 4: Additional CB1R/CB2R Activity and Beta-Arrestin Assay Data

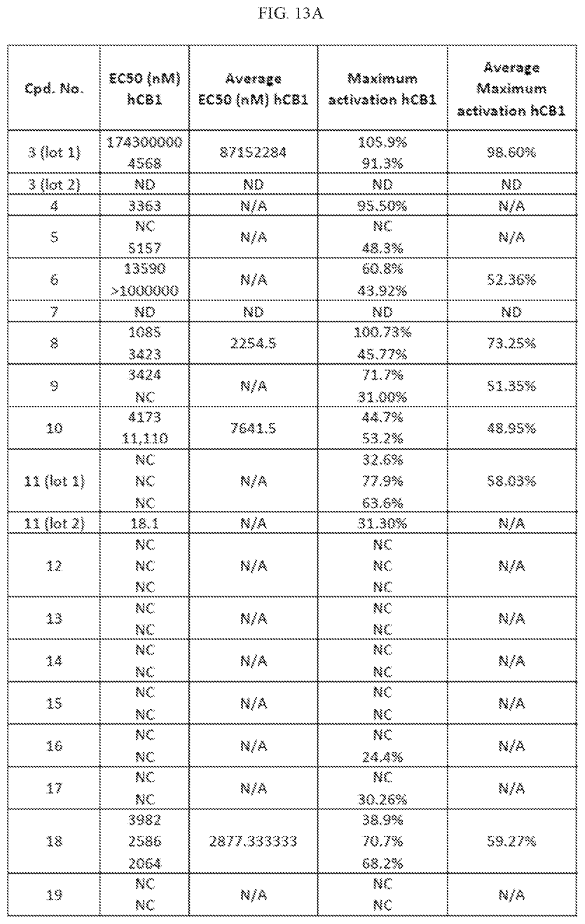

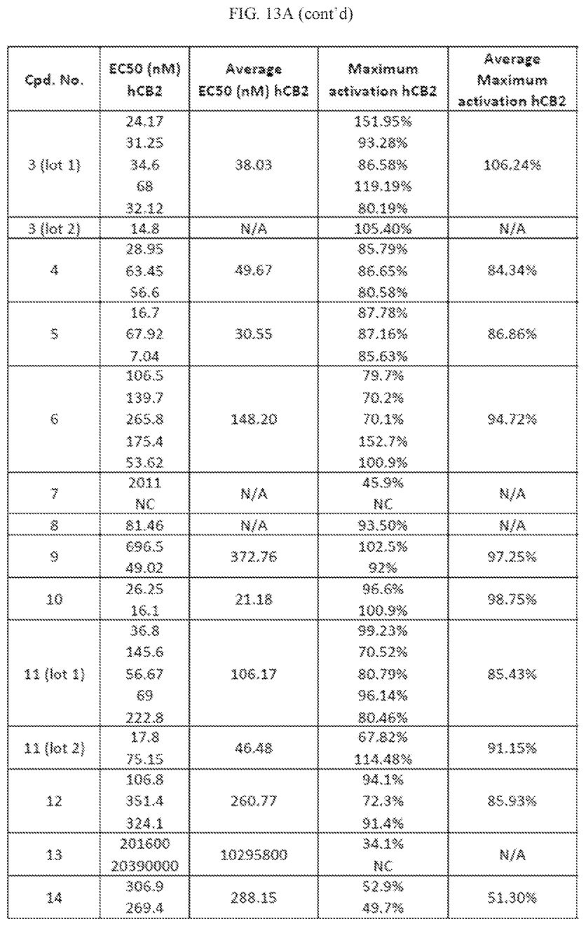

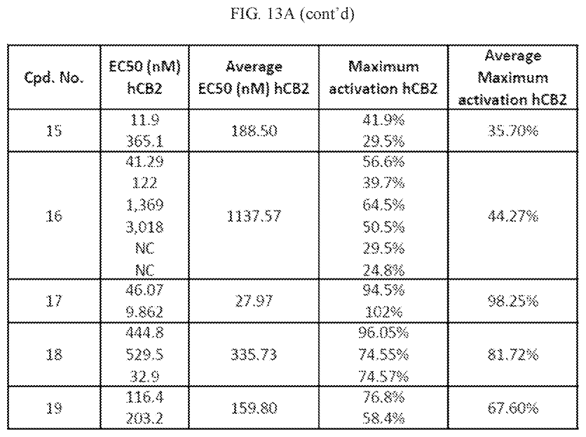

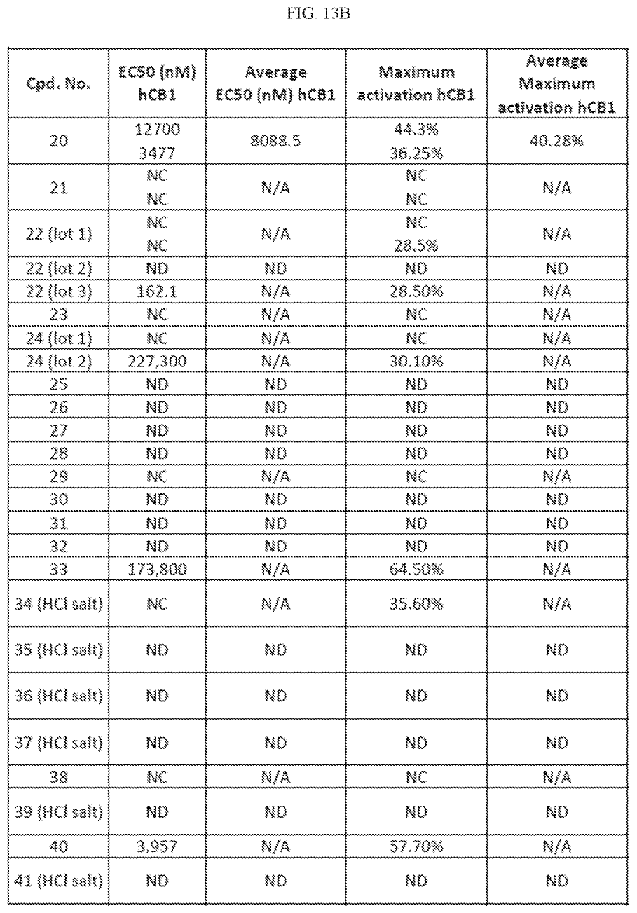

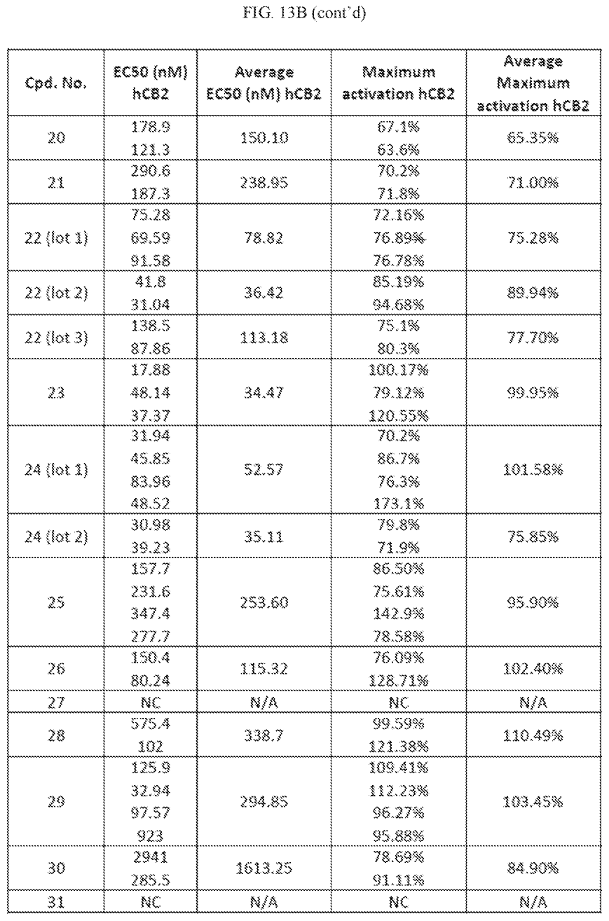

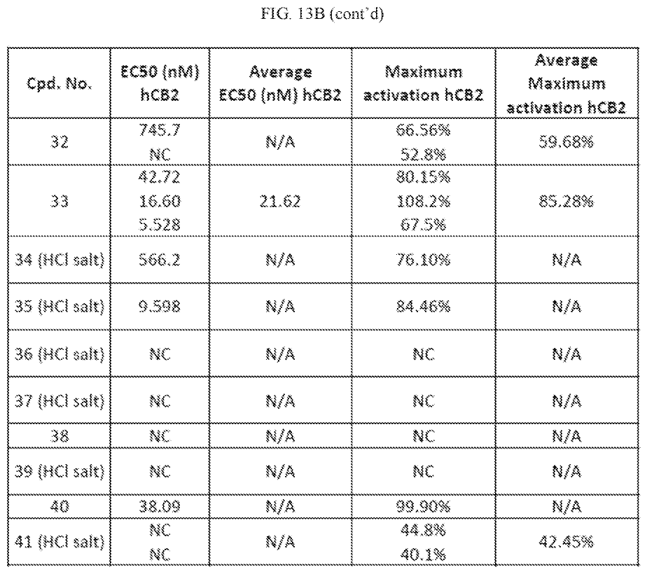

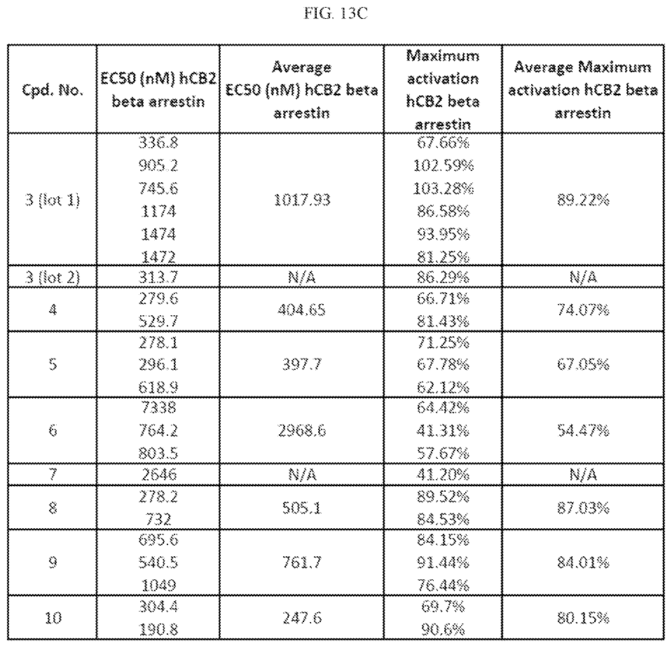

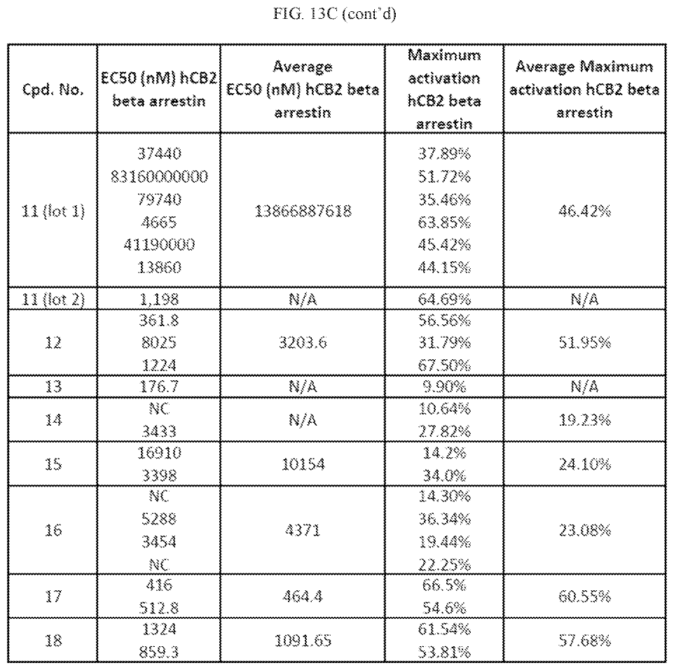

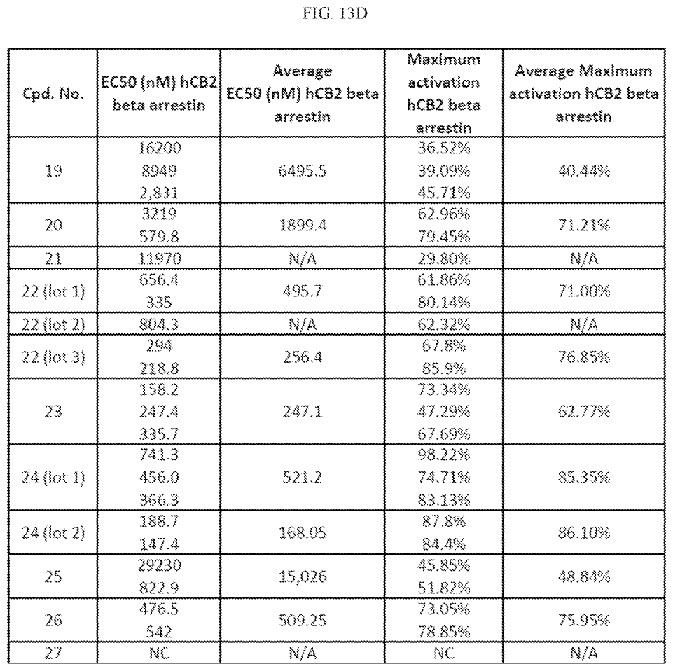

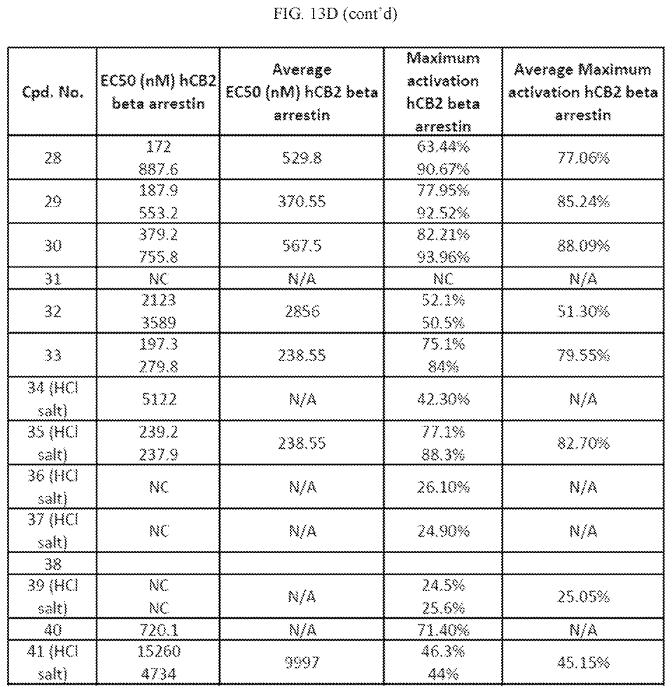

The procedures in Examples 2 and 3 were carried out for each compound and the data are presented in FIGS. 13A-13D. In these tables, each data point is the result of at least two independent experiments performed on the same day, with the results averaged. N/A=not applicable. ND=not determined. NC=data not converged.

### Example 5: In Vivo Efficacy Study—Heroin Self-Administration Model

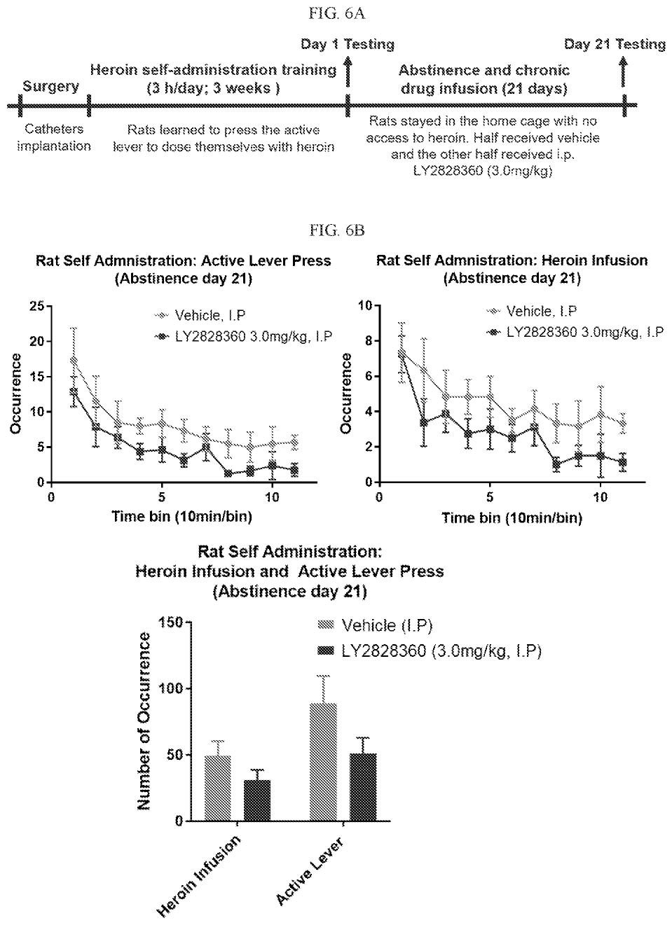

In the heroin SA model, rats were surgically implanted with a catheter that connects to their heart. Using special computerized cages containing two levers, an active lever and an inactive lever, rats were trained over 15 days in 3 hr sessions per day (FIG. 6A). They learned to press an active lever to get an infusion of heroin through their implanted tube. Pressing the inactive lever led to no consequences. Heroin SA-trained rats underwent 21 days of abstinence after training. During this period, half of the rats were treated daily with the CB2R agonist LY2828360, while half received vehicle. Heroin-seeking behaviors were assessed before and after the abstinence period. LY2828360-treated rats showed less abstinence-induced heroin seeking than the vehicle-treated rats; it seems that the selective CB2R agonist reduced their craving for heroin (FIG. 6B).

This experiment can be repeated using methamphetamine instead of heroin to evaluate reduction of methamphetamine-induced hyperactivity.

### Example 6: In Vivo Efficacy Study—Naloxone-Precipitated Withdrawal Model

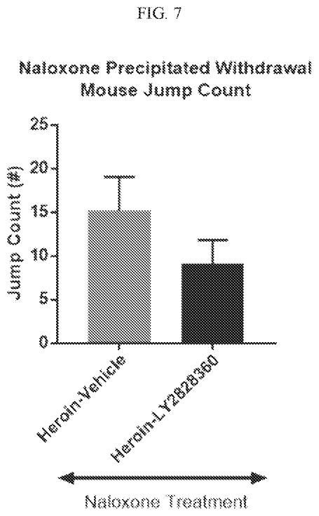

The naloxone-precipitated withdrawal model uses an opioid receptor antagonist, naloxone, to trigger highly aversive withdrawal symptoms (e.g., jumping, burrowing, “wet-dog” shakes, hyperreactivity) in heroin-addicted mice. Signs such as jumping can be quantified after administration of naloxone (“naloxone precipitated withdrawal”). LY2828360 (3 mg/kg, i.p.) or vehicle was administered on Days 1 and 2 followed by co-administration with each heroin dose for Days 3-6 (3 escalating heroin/drug doses a day at 10, 20, 40 mg/kg). On Day 7, 60 minutes after a final drug and heroin dose (40 mg/kg), naloxone was administered (50 mg/kg), and jump count measured for 1-15 minutes. LY2828360 treatment was observed to reduce the negative effects of heroin withdrawal in addicted mice, as seen by a reduction in “jumping” behavior 1 minute after naloxone administration (FIG. 7). These results suggest that targeting CB2R receptors can reduce addictive behavior and mitigate withdrawal symptoms in mice.

This experiment can be repeated using methamphetamine instead of heroin to evaluate reduction of negative effects of methamphetamine withdrawal in addicted mice.

### Example 7: In Vivo Efficacy Study—Conditioned Place Preference Model

In the Conditioned Place Preference (CPP) model, rats are placed in special cages containing two chambers, a black one with a textured floor, and a white one with a smooth floor. Rats receive an injection of heroin or methamphetamine immediately before being placed in the black chamber (with dividing wall in place), but receive a saline injection before being placed in the white chamber. Over time, rats learn to associate the black chamber with heroin or methamphetamine, and when allowed to freely move between chambers, demonstrate a preference for the drug-associated chamber. Once rats are trained to associate one compartment with drug injection (0.5 mg/kg, i.p.) and the other with saline (1 ml/kg, i.p.), then they receive vehicle or a CB2R agonist (e.g., a compound disclosed herein) (5 mg/kg, i.p.) (extinction session) before placement in the previously drug-associated context. It is expected that vehicle-treated rats will maintain a strong preference for the drug-associated chamber, while compound-treated rats will not.

### Example 8: General Pharmacokinetic, Safety, and Efficacy Studies

The P-glycoprotein (P-gp) inhibition assay will be used to assess potential drug-drug interactions (advancement criteria: <10% inhibition at 10 μM).

A microsomal stability assay using liver microsomes will assess in vitro stability (advancement criteria: half-life >60 min).

For maximum tolerated dose (MTD) determination, a high dose of compound (ca 50 mg/kg, i.p.) will be administered to 3 C57Bl/6J mice, and the animals will be closely monitored for any signs of toxicity. If no adverse effects are observed, the dose will be escalated until signs of toxicity are present. If the initial dose leads to observable toxicity, the quantity will be progressively reduced to determine a dose that has no adverse effects. Once the MTD is determined, 3 different doses will be administered daily for 14 days (n=3 per dose) using the 3-fold dose deduction protocol starting at the determined MTD. After 14 days of administration, any toxic effects will be identified using a comprehensive clinical chemistry panel and histopathology of vital organs.

For PK evaluation, drug will be administered i.p. to C57Bl/6J mice and Sprague-Dawley rats at 3 mg/kg dose. A 10-point PK curve will be generated by euthanizing animals at 3 min, 15 min, 30 min, 1 h, 2 h, 4 h, 6 h, 8 h, 12 h, and 24 h after administration of compound. Plasma and tissues including brain, heart, and liver will be collected at each time point. Concentrations of drug in plasma and processed tissue samples will be measured by LC-MS/MS. Total measured brain and plasma concentrations will be converted to unbound brain and plasma concentrations using the fraction unbound of brain and plasma measured in vitro. The PK data will be analyzed by non-compartmental methods to determine PK parameters including maximum plasma concentration (Cmax), time to maximum concentration (Tmax), half-life (t1/2), and area under curve (AUC). The brain:plasma ratio will be calculated with the unbound brain and plasma concentrations. Advancement criteria: half-life >120 min and brain/plasma ratio >0.2.

### Example 9: Hyperactivity Data

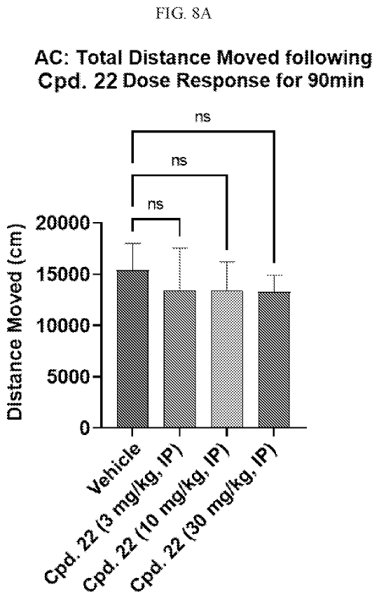

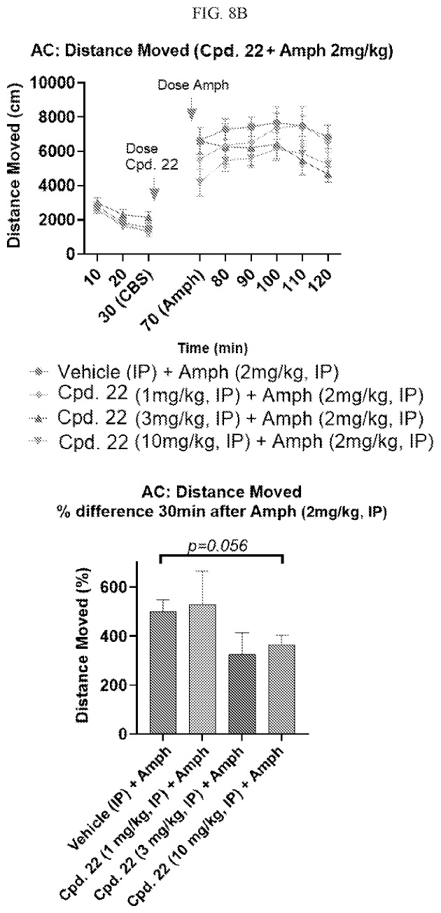

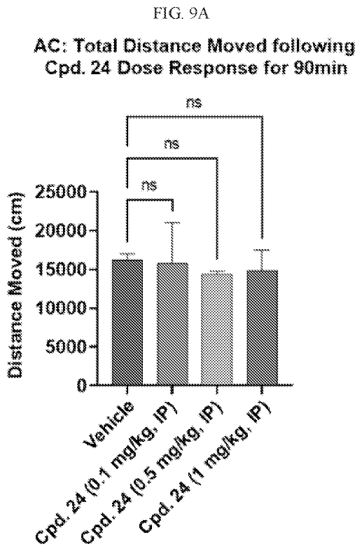

Male C57Bl6J mice were evaluated in an activity chamber apparatus for the amphetamine-induced hyperactivity test. Mice dosed with Compound 22 (N-(5-(1-hydroxy-2-methylpropan-2-yl)isoxazol-3-yl)-2-(4-(trifluoromethyl)phenyl)pyrazolidine-1-carboxamide) at 3, 10, and 30 mg/kg, i.p. do not exhibit significantly different distance moved compared to the vehicle for 90 minutes after dosing (see FIG. 8A). Thus, Compound 22 alone is neither stimulating nor sedating the mice, and the results further suggest a lack of any acute adverse behavioral effects. To determine the effects of Compound 22 on amphetamine-induced hyperactivity, mice were placed in the activity chamber for a 30 min baseline period. At the end of that period, mice were removed from the chamber, dosed intraperitoneally with Compound 22 (1 mg/kg, 3 mg/kg, or 10 mg/kg) or vehicle and returned to the home cage. Thirty minutes after dosing, mice were dosed (ip) with amphetamine (AMPH, 2 mg/kg) and immediately placed into the activity chamber for a one-hour post-dose session. Data are shown in FIG. 8B, and show that Compound 22 at 1 mg/kg, 3 mg/kg, or 10 mg/kg reduces the hyperactivity induced by AMPH compared to vehicle controls, with significant reduction at 10 mg/kg.

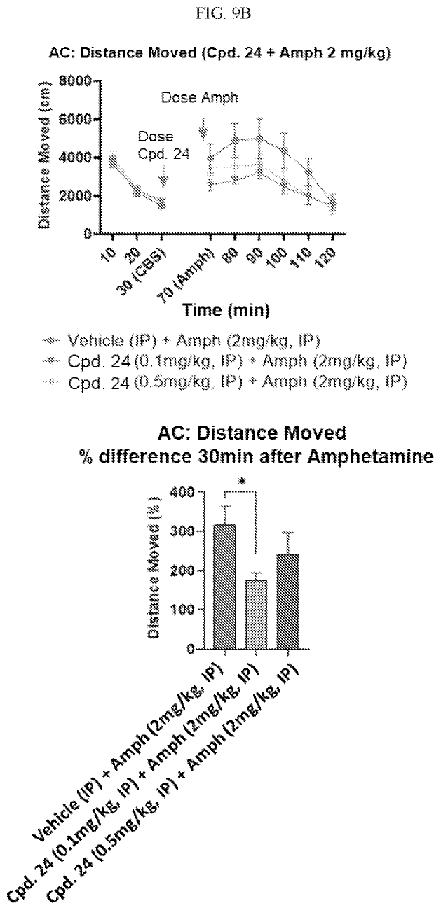

These experiments were repeated using Compound 24, and data are shown in FIGS. 9A and 9B. (% difference=[(Distance moved during first 30 min of Amphetamine/last 10 min of habituation]*100. *p=0.05; n=4-8/group. Data are means+/−SEM. 1-way ANOVA used for (A); unpaired t-test used for (B).) Compound 22 appears to be more potent in vivo than Compound 22, and is able to reduce hyperactivity at doses as low as 0.1 mg/kg, i.p.

### Example 10: Brain and Plasma Concentration Studies

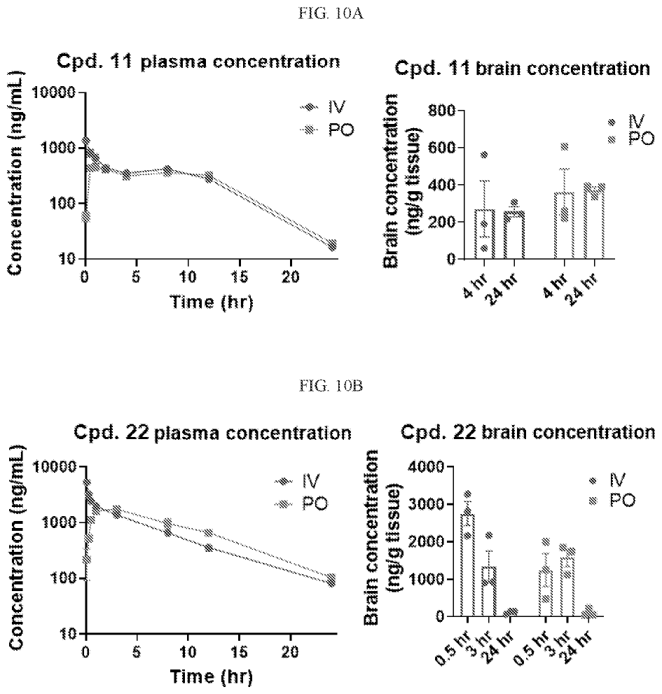

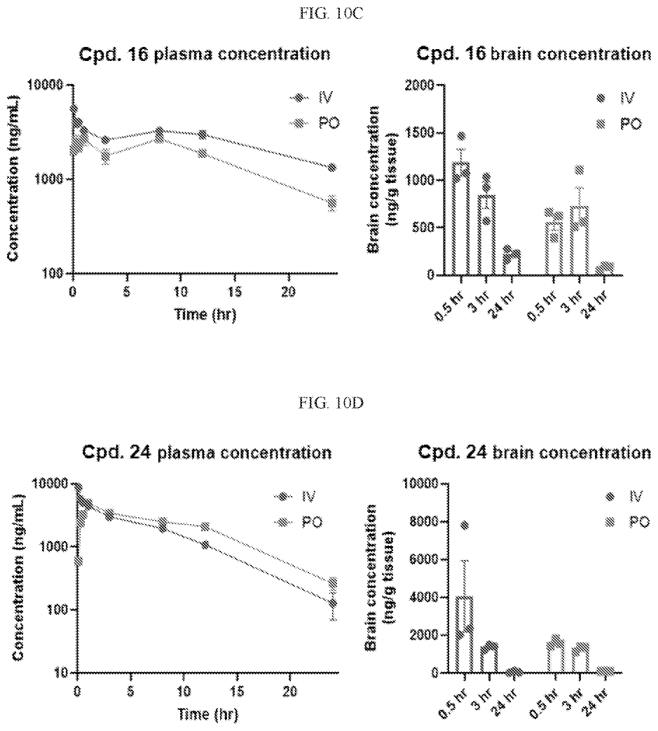

Plasma and brain concentrations of Compounds 11, 22, 16, and 24 were determined by LC-MS/MS after IV and PO dosing in male C57BL/6 mice at 10 mg/kg. Data are shown in FIGS. 10A-10D for Compounds 11, 22, 16, and 24, respectively.

### Example 11: Compound Profile Table for Compound 22

Detailed characterization data was collected for Compound 22. Primary assays were carried out according to Examples 2 and 3, while other assays were carried out according to standard methods. Data are shown in Table 6.

TABLE 6

| Category | Parameter | Activity | Comments |
|---|---|---|---|
| Chemistry | Molecular weight | 398.39 g/mol | |
| | Aqueous solubility | 17.36 μg/mL | |
| | SAR | 42 compounds tested | |
| Pharmacology | Activity in primary assay | CB2 EC50 67 nM | hCB1 & CB2 cAMP and |
| | | CB1 EC50 >10,000 nM | hCB2 beta-arrestin cell- |
| | | CB2 β-arrestin | based assays (agonist |
| | | EC50 578 nM | mode); EC50 values |
| | | | calculated from mean of |
| | | | n = 3-5 expts on ≥2 batches |
| | Activity in secondary assay | μM/M | In progress |
| | Selectivity (related family) | 150 fold selectivity | CB1/CB2 cAMP |
| | Selectivity (pathway) | 9 fold selectivity | CB2 cAMP/CB2 beta-arrestin |
| | Off-target activity | IC50 & EC50 >10 μM | GPR18, GPR55, GPR119 |
| | | | (agonist, antagonist, PAM modes) |
| | Activity in broad | No significant off | gpcrMAX at 70 nM against |
| | screening panel | target activity | 168 GPCRs completed |
| DMPK | Brain to plasma ratio | 0.97 | Via oral administration, 3 |
| | | | mice, at 24 h |
| | Bioavailability | 100% | Oral bioavailability |
| | In vitro permeability | High | Caco-2 |
| | P-glycoprotein transport | BAAB ratio | Not done |
| | Plasma protein binding | 93.69% | |
| | Brain protein binding | 98.02% | |
| | Microsomal stability | t ½ 120 min | Low clearance |
| | Mouse hepatocytes | >t ½, 120 min | Medium clearance |
| | metabolic stability | | |
| | P450 inhibition | | Not done |
| Toxicology | CYP induction | | Not done |
| | Cytotoxicity | | not toxic |
| | (Neurons/Microglia) | | |
| | Ames activity | | Not done |
| | hERG activity | IC50 >10 μM | |

### Example 12: Microdialysis Study

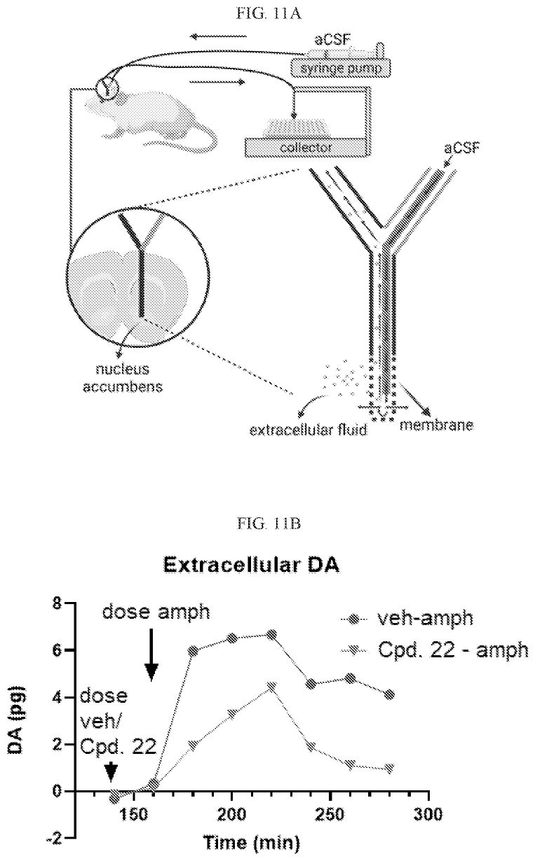

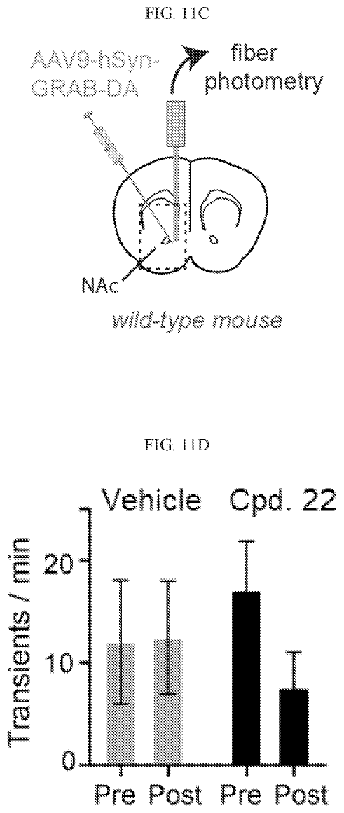

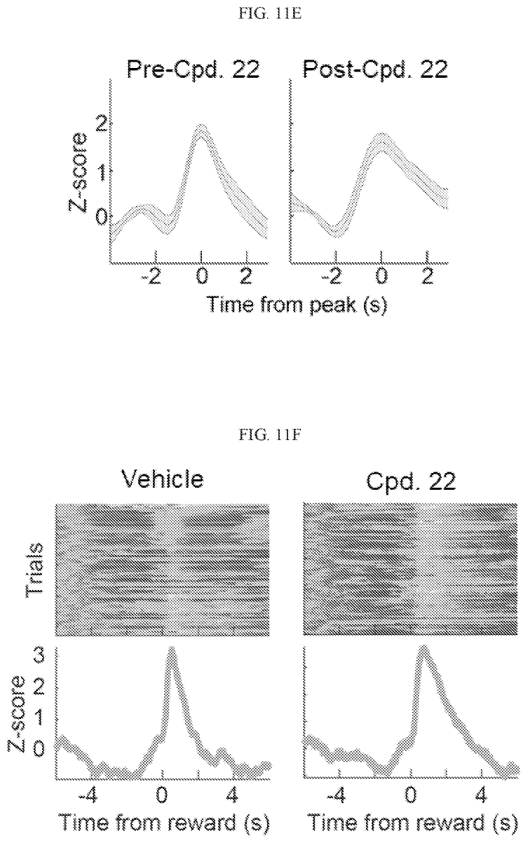

For this study, a probe was implanted into the nucleus accumbens (NAc) (FIG. 11A). Following baseline measurements, mice were dosed with vehicle or Compound 22 (10 mg/kg, i.p) followed 40 min later by a single dose of AMPH (2 mg/kg, i.p.). Samples were collected for 2 hr post-dosing. Extracellular dopamine (DA) levels in the dialysate were quantified by HPLC with electrochemical detection (FIG. 11B). The Compound 22 dosed animal shows reduced extracellular DA release after AMPH dosing. Compound 22 modulates both spontaneous and reward-related DA release. To confirm these findings in parallel experiments, GPCR-activation based-DA (GRAB-DA) was used, which is a human D2R-GFP chimera sensor that produces fluorescence changes reliably and with sub-second kinetics in response to extracellular DA concentration changes in behaving animals (Sun et al. Cell 2018; 174(2):481-96.e19). Mice were injected with an adeno-associated virus (AAV) carrying GRAB-DA into the (NAc) (FIG. 11C) and allowed to recover for −2 weeks before fiber photometry experiments were performed. In the first pilot experiment, baseline DA release were recorded for 5 min before mice (n=3) were injected with either Compound 22 or vehicle and then recording was continued for 45 min. The rate of spontaneous DA transients was compared in the pre-injection (0-5 min) vs post-injection (35-50 min) periods; there was a significant time x drug interaction: F(1,4)=11.91, P=0.026. Compound 22, but not the vehicle, reduced the rate of spontaneous transients (FIG. 11D). Compound 22 also appeared to affect the shape of the spontaneous transients, widening the curve (FIG. 11E).

In a second experiment, the same mice (n=3) were trained to nose poke a dedicated port to gain access to sucrose reward. During the task, GRAB-DA was used to measure DA release in response to reward. After ˜2 weeks of training, the effect of Compound 22 vs. vehicle on the DA response to reward was examined. On the first day, either drug or vehicle was injected followed by waiting 30 min, and then recording DA during the task. The order of drug vs. vehicle was counterbalanced between mice. While there was not a significant effect on the ability of mice to perform this task or the maximum reward-induced DA release, much like for the spontaneous transients, the drug appeared to widen the DA response curve (example in FIG. 11F). Thus, Compound 22 appears to be capable of modulating both spontaneous and reward-related DA release.

These experiments can be repeated by administering methamphetamine in place of AMPH.

### Example 13: Assays to Evaluate Efficacy in Parkinson's Disease

The effects of pharmacological activation of CB2 on microglia phagocytosis activity against α-syn will be evaluated. Human immortalized microglia cell line SV40 will be exposed to full-length human fibrillary α-synuclein at a concentration of 0, 0.1, 1, and 2.5 mM. TNF-α and IL-10 levels will be evaluated by qPCR and ELISA. Additionally, SV40 cells will be exposed to synaptosomal preparations containing fibrillary α-synuclein. Briefly, synaptosomes prepared from rat brains are conjugated with a pH-sensitive fluorophore (Phrodo™ Red) which will fluoresce once within the lysosomal lumen. Internalization of conjugated lysosomes is tracked by an IncuCyte instrument every 15 minutes for 7 hours.

The neuroprotective effects of the compounds will be evaluated against 6-OHDA toxicity in a reconstituted neuron-microglia co-culture. Cells will be treated with 6-OHDA for 24 hours at a concentration ranging from 1 μM to 1 mM to induce cell toxicity. CB2-dependent effects on SH-SY5Y cells will be evaluated with and without 6-OHDA neurotoxin. Cell Titer-Glo luminescent assay will assess cell viability and any neuroprotective effects of our CB2 agonists. Pro- and anti-inflammatory markers such as IL-10, TNF-α, IL-6, and IL-10 will be investigated via qPCR and multiplex assay for all 48 cytokines.

Two separate mouse models of PD will also be used. First, the neuroprotective effects of the compounds in a 6-OHDA mouse model of PD will be evaluated. In brief, 20 μg of 6-OHDA will be intracranially infused by convection-enhanced delivery into the striatum of male mice C57BL6/J 10 weeks of age. Such intervention will selectively injure dopaminergic neurons of the nigrostriatal system over a two-week period. Lesion extent will be quantified using biochemical and histological techniques in order to identify any effects deriving from the use of our novel CB2 agonists against 6-OHDA neurotoxicity. Second, the effects of the CB2 agonist on the prion-like spreading of α-synuclein pathology will be evaluated both within and outside the nigrostriatal system. C-terminally α-syn (1-121) is an aggregation-prone species resulting from caspase-1 proteolysis. α-syn(1-121) will be used as the fibrillary seed to trigger α-synuclein pathology in WT C57BL6/J. In brief, mice will be subjected to stereotaxic deposition of h-α-syn(1-121) seeded into the neostriatum. The course of α-synuclein aggregation will be evaluated at 1, 3, and 6 months in the vehicle and CB2 agonist-treated mice. Several biochemical and histological endpoints will evaluate the prion-like spreading of the α-synuclein pathology, which will also be used to assess the therapeutic efficacy of our CB2 agonists by determining α-synuclein load microglia activation, and dopaminergic lesion. 3D brain clearing and scanning coupled with a machine learning protocol developed in our lab will help us to process a large amount of data in an expedited manner. In addition, flow-cytometry assessment of single-cell suspensions obtained from the brain parenchyma will allow assessment of any changes in the infiltration of immune cells in specific brain regions.

### Example 14: Evaluation of Anti-Inflammatory Properties

Mice were dosed with Compound 24 (0.5 mg/kg ip) or vehicle daily for 10 days. Lipopolysaccharide (LPS) (100 ug/kg ip) was given 1 hour after 10^{th} dose on final day. ATP (50 mg/kg ip) was given 2 hours after LPS and then mice were sacrificed 30 minutes after ATP (2.5 hours after LPS). Multiplex tissue cytokines were analyzed in plasma and brain homogenate from all brain regions dissected above using a Luminex 38-plex mouse cytokine assay as previously described for aged versus young mice (Evans et al. Neurobiol Aging 2021; 106:241-56). The Luminex assay was performed according to the manufacturer's instructions. Briefly, tissue samples were homogenized in RIPA buffer containing proteinase inhibitor by pulling tissue through a 23 g needle (15×) and then sonicating for 3×3 second pulses. Homogenate was spun at 14,000 g for 10 minutes, and protein concentrations were determined by the Pierce BCA assay. Samples were diluted to a common concentration of 6 μg/μL. Plasma samples were diluted 1:3. Plasma and brain homogenate samples were run in duplicate on a 96 well plate alongside standard curve and quality control calibration samples.

Male C57BL/6J mice, 6-8 weeks old were dosed on Day 1,2,3,4,5, and 10, or 11 with vehicle or CBS-159 at 0.1 and 0.3 mg/kg or 1, 2, and 3 mg/kg. Mice were terminated on Day 10 or 11, 3.5 hours after dosing vehicle or CBS-159. Total RNA was isolated from brain tissue dissections using the RNeasy Lipid Tissue Mini Kit (Qiagen) (see Evans et al. Neurobiol. Dis. 2020, 146:105089). One microgram of total RNA was transcribed into cDNA (Superscript III, Invitrogen). PCR was performed in triplicate using TaqMan gene expression master mix (Applied Bio-systems) and validated TaqMan gene expression assays, Tnfa (Mm00443258_m1), IL1β (Mm00434228_m1), IL6 (Mm00446190_m1),), IL18 (Mm00434226_m1), and glyceraldehyde-3-phosphate dehydrogenase (Gapdh; Mm99999915_g1). Amplification was performed using a StepOnePlus system (Applied Biosystems). Fold changes of expression relative to control were determined after normalization to Gapdh. Relative quantification and old change were calculated by the comparative CT method (Schmittgen and Livak, Nat. Protoc. 2008, 3(6):1101-1108).

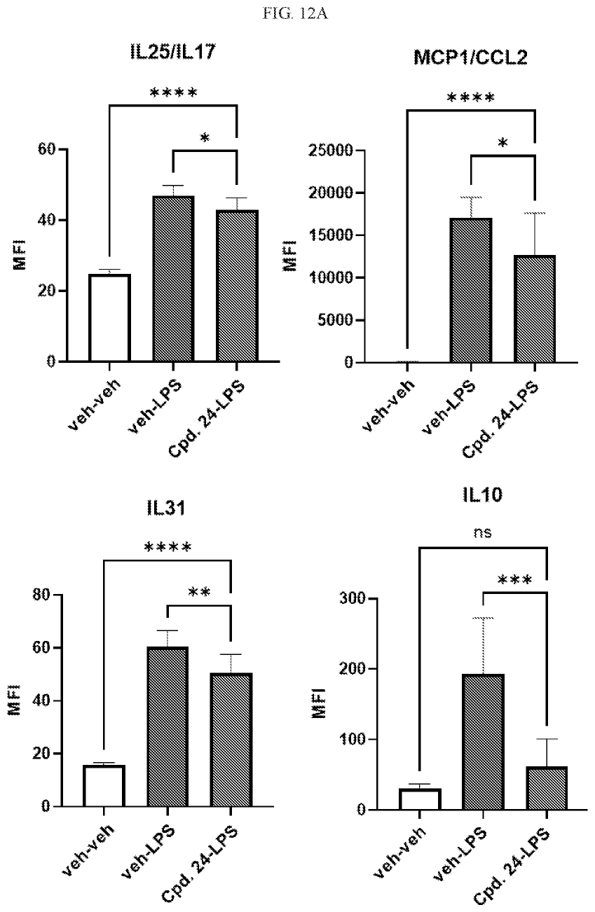

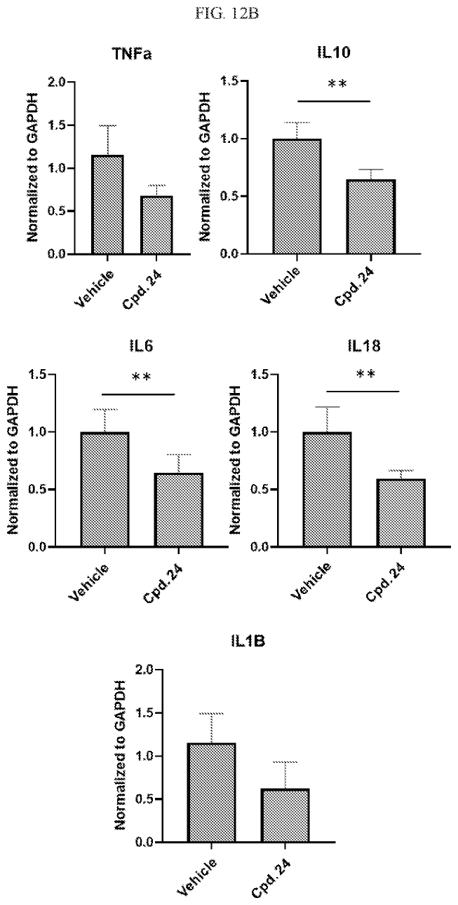

Data are shown in FIGS. 12A and 12B, and show that Compound 24 has anti-inflammatory activity.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A compound of formula (I): 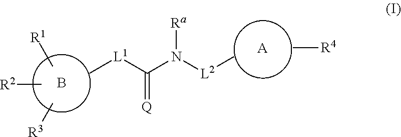
or a pharmaceutically acceptable salt thereof, wherein:
Q is O, S, or NR^{b};
L¹ is a bond or CR^{c}R^{d};
L² is a bond or CR^{e}R^{f};
A is a five- or six-membered heteroaryl having 1, 2, or 3 heteroatoms independently selected from N, O, and S; or a 8- to 10-membered heterocyclyl having 1, 2, or 3 heteroatoms independently selected from N, O, and S; wherein the heteroaryl or the heterocyclyl is optionally substituted with one substituent selected from C₁₋₆ alkyl and C₁₋₆ haloalkyl;
B is a 5- or 6-membered monocyclic heterocyclyl, a 5- or 6-membered monocyclic heteroaryl, or an 8- to 10-membered bicyclic heterocyclyl;
R¹, R², R³, R⁴, and R⁵ are each independently selected from hydrogen, C₁₋₆ alkyl, halo-C₁₋₆-alkyl, C₃₋₇ Cycloalkyl, C₁₋₃-alkyl-C₃₋₇-Cycloalkyl, C₃₋₇-Cycloalkyl-C₁₋₃-alkyl, —C(O)—C₁₋₆ alkyl, —C(O)-heterocyclyl, —NR^{6a}R^{6b}, —NR^{6c}C(O)R^{6d}, oxo, aryl, arylalkyl, heteroaryl, and heterocyclyl, each of which is unsubstituted or substituted with 1-6 substituents selected from halo, halo-C₁₋₃-alkyl, cyano, C₃₋₇ cycloalkyl, —OR^{6e}, —(C₁₋₃ alkyl)-OR⁶f, —NR^{6g}R^{6h}, —C(O)NR⁶ⁱR^{6j}, and pentafluorosulfanyl;
R^{a}, R^{b}, R^{c}, R^{d}, R^{e}, and R^{f} are each independently selected from hydrogen and C₁₋₃ alkyl;
R^{6a}, R^{6b}, R^{6c}, R^{6d}, R^{6e}, R^{6f}, R^{6g}, R^{6h}, R⁶ⁱ, and R^{6j} are each independently selected from hydrogen, C₁₋₆ alkyl, and C₃₋₇ cycloalkyl.

2. The compound of claim 1, or a pharmaceutically acceptable salt thereof, wherein A is a five-membered heteroaryl having 1, 2, or 3 heteroatoms independently selected from N, O, and S.

3. The compound of claim 1, or a pharmaceutically acceptable salt thereof, wherein the group 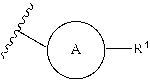
has a formula selected from: 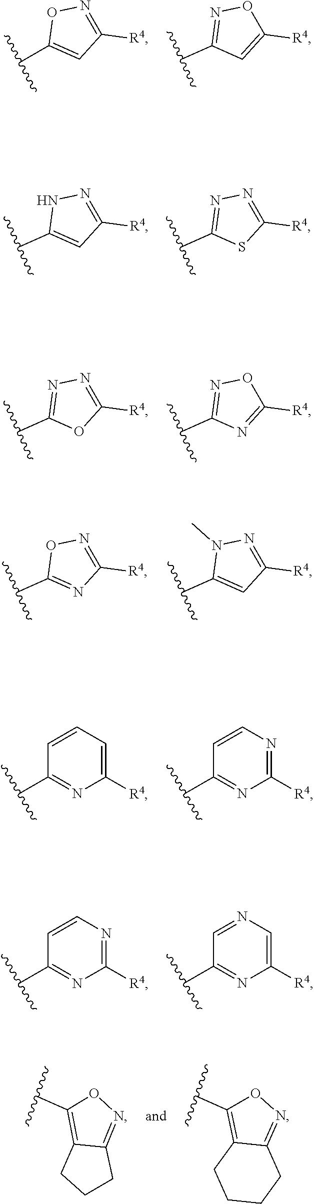
wherein [symbol] represents the point of attachment to L² in formula (I).

4. The compound of claim 1, or a pharmaceutically acceptable salt thereof, wherein R⁴ is selected from C₁₋₆ alkyl, halo-C₁₋₆-alkyl, C₃₋₇ cycloalkyl, C₁₋₃-alkyl-C₃₋₇-cycloalkyl, aryl, —NR^{6a}R^{6b}, —NR^{6c}C(O)R^{6d}, and heterocyclyl, each of which is unsubstituted or substituted with 1-4 substituents independently selected from halo, —OR⁶e, —(C₁₋₃ alkyl)-OR^{6f}, —NR^{6g}R^{6h}, and cyano, wherein R^{6e}, R^{6f}, R^{6g}, and R^{6h} are each independently selected from hydrogen and methyl.

5. The compound of claim 1, or a pharmaceutically acceptable salt thereof, wherein R⁴ is selected from:

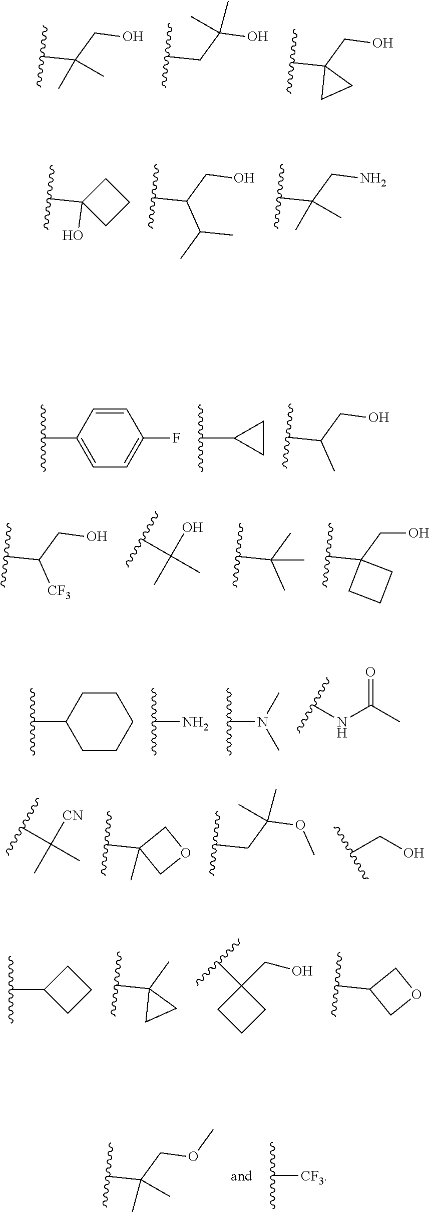

6. The compound of claim 1, or a pharmaceutically acceptable salt thereof, wherein R^{a} is hydrogen.

7. The compound of claim 1, or a pharmaceutically acceptable salt thereof, wherein Q is O or NR^{b}, wherein R^{b} is selected from hydrogen and methyl.

8. The compound of claim 1, or a pharmaceutically acceptable salt thereof, wherein L¹ and L² are each independently selected from a bond and —C(CH₃)₂—.

9. The compound of claim 1, or a pharmaceutically acceptable salt thereof, wherein B is a monocyclic 5- or 6-membered heterocyclyl having 1 or 2 nitrogen atoms, or a bicyclic 8- to 10-membered heterocyclyl having 1 or 2 nitrogen atoms.

10. The compound of claim 1, or a pharmaceutically acceptable salt thereof, wherein R² is selected from hydrogen and oxo.

11. The compound of claim 1, or a pharmaceutically acceptable salt thereof, wherein R³ is selected from hydrogen, C₁₋₆ alkyl, —C(O)CH₃, and —C(O)N(CH₃)₂.

12. The compound of claim 1, or a pharmaceutically acceptable salt thereof, wherein the group 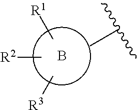
has a structure selected from: 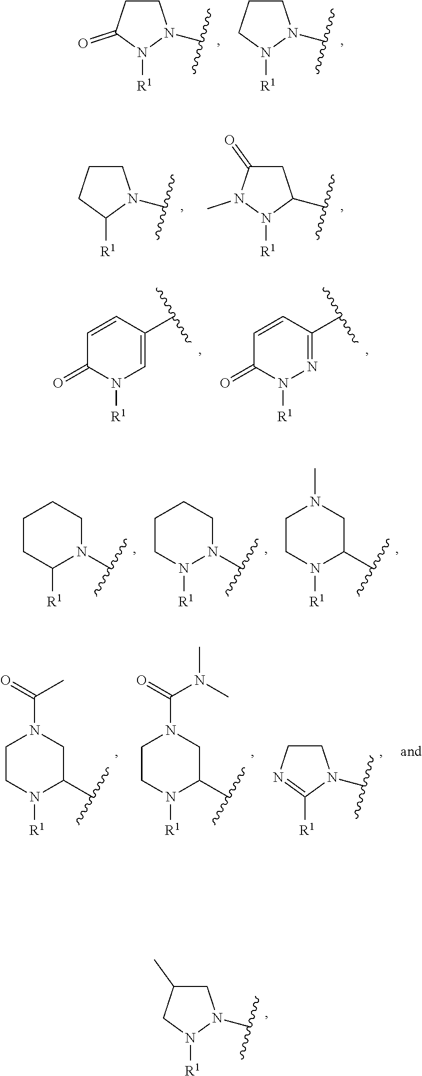
wherein [symbol] represents the point of attachment to L¹ in formula (I).

13. The compound of claim 1, or a pharmaceutically acceptable salt thereof, wherein R¹ is selected from aryl, heteroaryl, C₃₋₇ cycloalkyl, C₁₋₆ alkyl, C₃₋₇-cycloalkyl-C₁₋₃-alkyl, arylalkyl, heterocyclyl, and —C(O)heterocyclyl, each of which is independently unsubstituted or substituted with 1-3 substituents independently selected from halo, halo-C₁₋₃-alkyl, cyano, C₃₋₇ cycloalkyl, and pentafluorosulfanyl.

14. The compound of claim 1, or a pharmaceutically acceptable salt thereof, wherein R¹ is selected from:

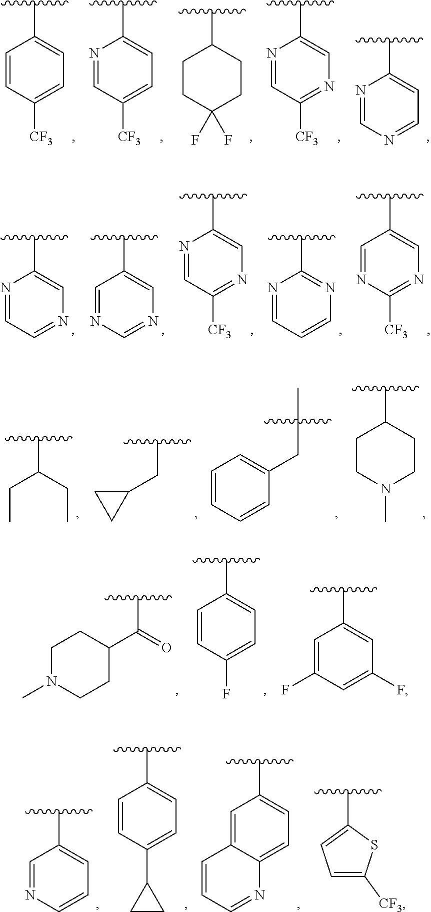

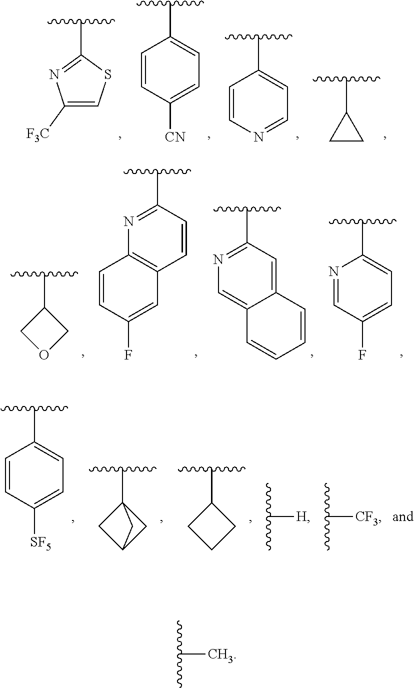

15.-29. (canceled)

30. A compound selected from the group consisting of: 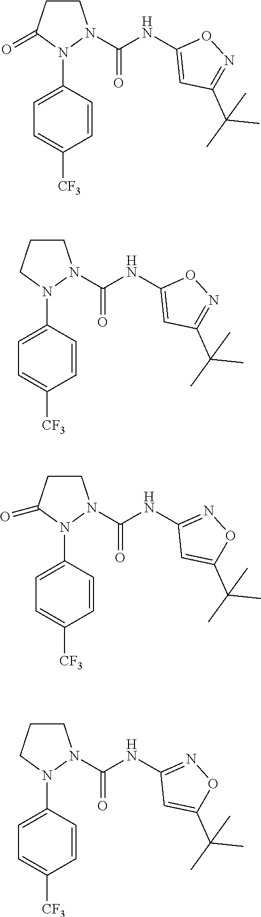 [SMILES: CC(C)(C)c1cc(NC(=O)N2CCC(=O)N2c2ccc(C(F)(F)F)cc2)on1 ; CC(C)(C)c1cc(NC(=O)N2CCCN2c2ccc(C(F)(F)F)cc2)on1 ; CC(C)(C)c1cc(NC(=O)N2CCC(=O)N2c2ccc(C(F)(F)F)cc2)no1 ; CC(C)(C)c1cc(NC(=O)N2CCCN2c2ccc(C(F)(F)F)cc2)no1] 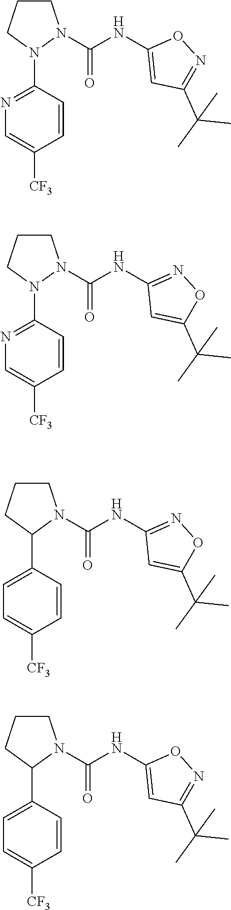 [SMILES: CC(C)(C)c1cc(NC(=O)N2CCCN2c2ccc(C(F)(F)F)cn2)on1 ; CC(C)(C)c1cc(NC(=O)N2CCCN2c2ccc(C(F)(F)F)cn2)no1 ; CC(C)(C)c1cc(NC(=O)N2CCCC2c2ccc(C(F)(F)F)cc2)no1 ; CC(C)(C)c1cc(NC(=O)N2CCCC2c2ccc(C(F)(F)F)cc2)on1] 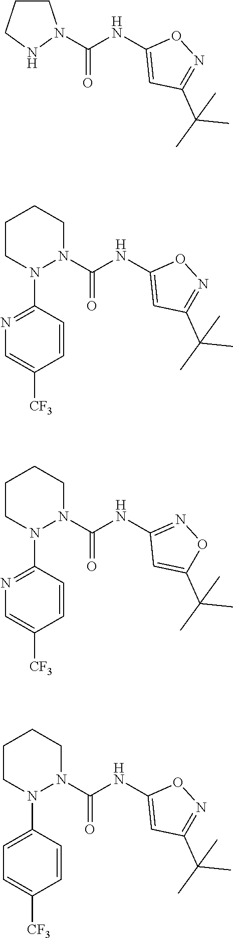 [SMILES: CC(C)(C)c1cc(NC(=O)N2CCCN2)on1 ; CC(C)(C)c1cc(NC(=O)N2CCCCN2c2ccc(C(F)(F)F)cn2)on1 ; CC(C)(C)c1cc(NC(=O)N2CCCCN2c2ccc(C(F)(F)F)cn2)no1 ; CC(C)(C)c1cc(NC(=O)N2CCCCN2c2ccc(C(F)(F)F)cc2)on1] 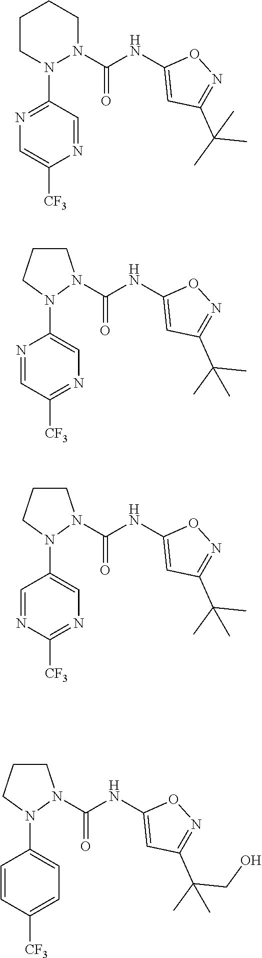 [SMILES: CC(C)(C)c1cc(NC(=O)N2CCCCN2c2cnc(C(F)(F)F)cn2)on1 ; CC(C)(C)c1cc(NC(=O)N2CCCN2c2cnc(C(F)(F)F)cn2)on1 ; CC(C)(C)c1cc(NC(=O)N2CCCN2c2cnc(C(F)(F)F)nc2)on1 ; CC(C)(CO)c1cc(NC(=O)N2CCCN2c2ccc(C(F)(F)F)cc2)on1] 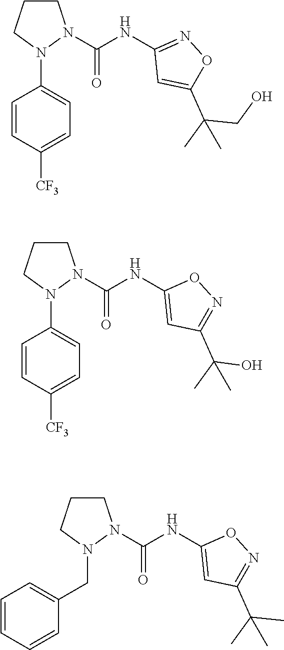 [SMILES: CC(C)(CO)c1cc(NC(=O)N2CCCN2c2ccc(C(F)(F)F)cc2)no1 ; CC(C)(O)c1cc(NC(=O)N2CCCN2c2ccc(C(F)(F)F)cc2)on1 ; CC(C)(C)c1cc(NC(=O)N2CCCN2Cc2ccccc2)on1] 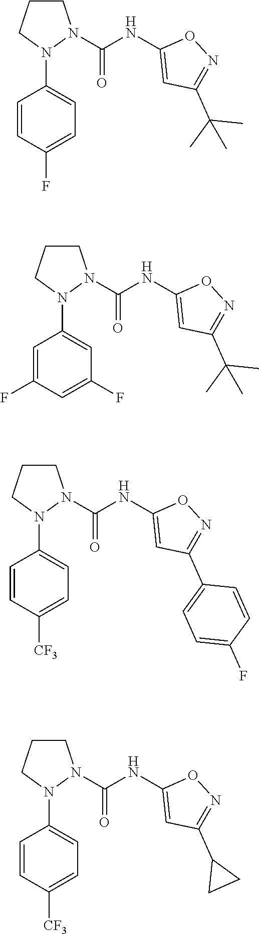 [SMILES: CC(C)(C)c1cc(NC(=O)N2CCCN2c2ccc(F)cc2)on1 ; CC(C)(C)c1cc(NC(=O)N2CCCN2c2cc(F)cc(F)c2)on1 ; O=C(Nc1cc(-c2ccc(F)cc2)no1)N1CCCN1c1ccc(C(F)(F)F)cc1 ; O=C(Nc1cc(C2CC2)no1)N1CCCN1c1ccc(C(F)(F)F)cc1] 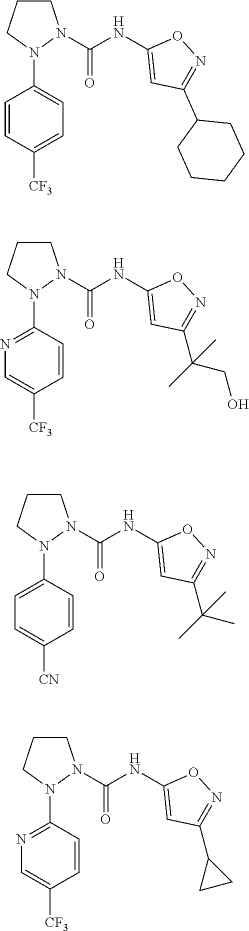 [SMILES: O=C(Nc1cc(C2CCCCC2)no1)N1CCCN1c1ccc(C(F)(F)F)cc1 ; CC(C)(CO)c1cc(NC(=O)N2CCCN2c2ccc(C(F)(F)F)cn2)on1 ; CC(C)(C)c1cc(NC(=O)N2CCCN2c2ccc(C#N)cc2)on1 ; O=C(Nc1cc(C2CC2)no1)N1CCCN1c1ccc(C(F)(F)F)cn1] 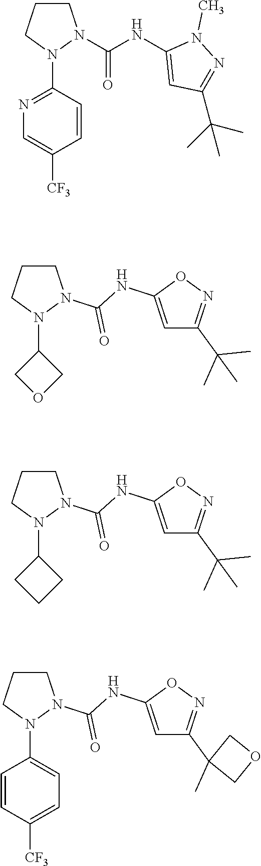 [SMILES: Cn1nc(C(C)(C)C)cc1NC(=O)N1CCCN1c1ccc(C(F)(F)F)cn1 ; CC(C)(C)c1cc(NC(=O)N2CCCN2C2COC2)on1 ; CC(C)(C)c1cc(NC(=O)N2CCCN2C2CCC2)on1 ; CC1(c2cc(NC(=O)N3CCCN3c3ccc(C(F)(F)F)cc3)on2)COC1] 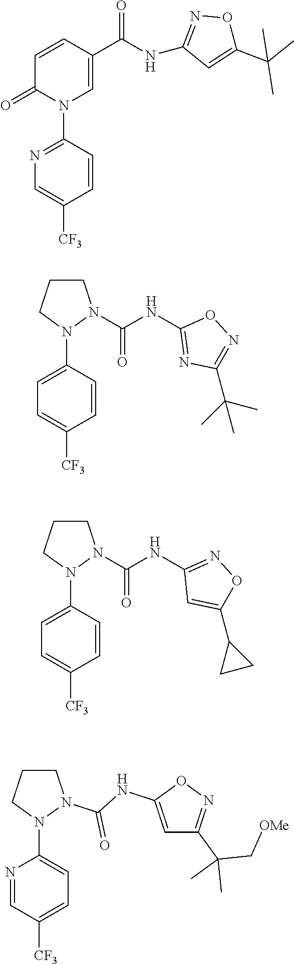 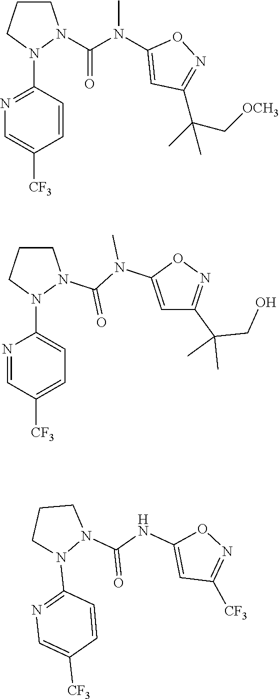 [SMILES: COCC(C)(C)c1cc(N(C)C(=O)N2CCCN2c2ccc(C(F)(F)F)cn2)on1 ; CN(C(=O)N1CCCN1c1ccc(C(F)(F)F)cn1)c1cc(C(C)(C)CO)no1 ; O=C(Nc1cc(C(F)(F)F)no1)N1CCCN1c1ccc(C(F)(F)F)cn1]
and pharmaceutically acceptable salts thereof.

31. A pharmaceutical composition comprising a compound of claim 1, or a pharmaceutically acceptable salt thereof, and a pharmaceutically acceptable carrier.

32. A method of selectively agonizing a cannabinoid 2 receptor in a subject, comprising administering to the subject an effective amount of a compound of claim 1, or a pharmaceutically acceptable salt thereof.

33. A method of treating a disorder in a subject in need of treatment, wherein the disorder is selected from addiction, pain, an inflammatory disorder or other disorder having an inflammatory component, a disease having a neuroinflammatory or neurodegenerative component, Alzheimer's disease, and Parkinson's disease, comprising administering to the subject a therapeutically effective amount of a compound of claim 1, or a pharmaceutically acceptable salt thereof.

34.-37. (canceled)

38. A method of modulating dopaminergic signaling in a subject in need thereof, comprising administering to the subject an effective amount of a compound of claim 1, or a pharmaceutically acceptable salt thereof.

39.-70. (canceled)

71. The method of claim 33, wherein the compound exhibits biased agonist activity for the CB2 receptor cyclase pathway or for the CB2 receptor arrestin pathway.
